SEQUENCE LISTING (138 sequences)

SEQ ID NO: 1 - DNA, 654 bp, synthetic construct
  source 1..654: mol_type = other DNA; note = Synthesized
cagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaagctgaccgtcctacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 2 - DNA, 333 bp, synthetic construct
  source 1..333: mol_type = other DNA; note = Synthesized
cagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaagctgaccgtccta

SEQ ID NO: 3 - protein, 218 aa, synthetic construct
  source 1..218: note = Synthesized
QSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 110 aa, synthetic construct
  source 1..110: note = Synthesized
QSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKLTV

SEQ ID NO: 5 - DNA, 1344 bp, synthetic construct
  source 1..1344: mol_type = other DNA; note = Synthesized
caggtgcagctgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 6 - DNA, 354 bp, synthetic construct
  source 1..354: mol_type = other DNA; note = Synthesized
caggtgcagctgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagc

SEQ ID NO: 7 - protein, 448 aa, synthetic construct
  source 1..448: note = Synthesized
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 118 aa, synthetic construct
  source 1..118: note = Synthesized
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 9 - protein, 642 aa, synthetic construct
  source 1..642: note = Synthesized
GACATCCAGATGACCCAGTCTCCATCCTCCCTGTCTGCATCTGTAGGAGACAGAGTCACCATCACTTGCCAGGCGAGTCAGGACATCAGCAACTATTTAAATTGGTATCAGCAGAAACCAGGGAAAGCCCCTAAACTCCTGATCTACGATGCATCCAATTTGGAAACAGGGGTCCCATCAAGGTTCAGTGGAAGTGGATCTGGGACAGATTTTACTTTCACCATCAGCAGCCTGCAGCCTGAAGATATTGCAACATATTTCTGTCAACACTTTGATCATCTCCCGCTCGCTTTCGGCGGAGGGACCAAGGTGGAAATTAAACGTACGGTGGCTGCACCATCTGTCTTCATCTTCCCGCCATCTGATGAGCAGTTGAAATCTGGAACTGCCTCTGTTGTGTGCCTGCTGAATAACTTCTATCCCAGAGAGGCCAAAGTACAGTGGAAGGTGGATAACGCCCTCCAATCGGGTAACTCCCAGGAGAGTGTCACAGAGCAGGACAGCAAGGACAGCACCTACAGCCTCAGCAGCACCCTGACGCTGAGCAAAGCAGACTACGAGAAACACAAAGTCTACGCCTGCGAAGTCACCCATCAGGGCCTGAGCTCGCCCGTCACAAAGAGCTTCAACAGGGGAGAGTGT

SEQ ID NO: 10 - DNA, 321 bp, synthetic construct
  source 1..321: mol_type = other DNA; note = Synthesized
gacatccagatgacccagtctccatcctccctgtctgcatctgtaggagacagagtcaccatcacttgccaggcgagtcaggacatcagcaactatttaaattggtatcagcagaaaccagggaaagcccctaaactcctgatctacgatgcatccaatttggaaacaggggtcccatcaaggttcagtggaagtggatctgggacagattttactttcaccatcagcagcctgcagcctgaagatattgcaacatatttctgtcaacactttgatcatctcccgctcgctttcggcggagggaccaaggtggaaattaaa

SEQ ID NO: 11 - protein, 214 aa, synthetic construct
  source 1..214: note = Synthesized
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 107 aa, synthetic construct [sequence omitted]

SEQ ID NO: 13 - DNA, 1347 bp, synthetic construct
  source 1..1347: mol_type = other DNA; note = Synthesized
caggtgcagctgcaggagtcgggcccaggactggtgaagccttcggagaccctgtccctcacctgcactgtctctggtggctccgtcagcagtggtgattactactggacctggatccggcagtccccagggaagggactggagtggattggacacatctattacagtgggaacaccaattataacccctccctcaagagccgactcaccatatcaattgacacgtccaagactcagttctccctgaagctgagttctgtgaccgctgcggacacggccatttattactgtgtgcgagatcgagtgactggtgcttttgatatctggggccaagggacaatggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 14 - DNA, 357 bp, synthetic construct
  source 1..357: mol_type = other DNA; note = Synthesized
caggtgcagctgcaggagtcgggcccaggactggtgaagccttcggagaccctgtccctcacctgcactgtctctggtggctccgtcagcagtggtgattactactggacctggatccggcagtccccagggaagggactggagtggattggacacatctattacagtgggaacaccaattataacccctccctcaagagccgactcaccatatcaattgacacgtccaagactcagttctccctgaagctgagttctgtgaccgctgcggacacggccatttattactgtgtgcgagatcgagtgactggtgcttttgatatctggggccaagggacaatggtcaccgtctcgagc

SEQ ID NO: 15 - protein, 449 aa, synthetic construct
  source 1..449: note = Synthesized
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 119 aa, synthetic construct [sequence omitted]

SEQ ID NO: 17 - DNA, 642 bp, synthetic construct
  source 1..642: mol_type = other DNA; note = Synthesized
gatatccagatgacccagtccccgagctccctgtccgcctctgtgggcgatagggtcaccatcacctgccgtgccagtcagaatattgctactgatgtagcctggtatcaacagaaaccaggaaaagctccgaagcttctgatttactcggcatccttcctctactctggagtcccttctcgcttctctggtagcggttccgggacggatttcactctgaccatcagcagtctgcagccggaagacttcgcaacttattactgtcagcaaagtgagccggagccgtacacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 18 - DNA, 321 bp, synthetic construct
  source 1..321: mol_type = other DNA; note = Synthesized
gatatccagatgacccagtccccgagctccctgtccgcctctgtgggcgatagggtcaccatcacctgccgtgccagtcagaatattgctactgatgtagcctggtatcaacagaaaccaggaaaagctccgaagcttctgatttactcggcatccttcctctactctggagtcccttctcgcttctctggtagcggttccgggacggatttcactctgaccatcagcagtctgcagccggaagacttcgcaacttattactgtcagcaaagtgagccggagccgtacacgttcggacagggtaccaaggtggagatcaaa

SEQ ID NO: 19 - protein, 214 aa, synthetic construct
  source 1..214: note = Synthesized
DIQMTQSPSSLSASVGDRVTITCRASQNIATDVAWYQQKPGKAPKLLIYSASFLYSGVPSRFSGSGSGTDFTLTISSLQPEDFATYYCQQSEPEPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 107 aa, synthetic construct [sequence omitted]

SEQ ID NO: 21 - DNA, 1353 bp, synthetic construct
  source 1..1353: mol_type = other DNA; note = Synthesized
gaggtgcagctggtcgagtctggcggcggactggtgcagcccggtggctctctgcgactgtcttgtgccgcctctggcttcaccctcagtggcgattggatccactgggtgcgacaggctcccggaaagggcctggagtgggtgggtgagatctctgctgccggtggctacaccgattacgccgactctgtgaagggccgattcaccatctctgccgacacctctaagaacaccgcctacctgcagatgaactctctgcgagccgaggacaccgctgtgtactactgtgcacgagaaagtagagtttccttcgaagccgccatggactactggggccagggcaccctggtgaccgtgtcctctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 22 - protein, 363 aa, synthetic construct
  source 1..363: note = Synthesized
GAGGTGCAGCTGGTCGAGTCTGGCGGCGGACTGGTGCAGCCCGGTGGCTCTCTGCGACTGTCTTGTGCCGCCTCTGGCTTCACCCTCAGTGGCGATTGGATCCACTGGGTGCGACAGGCTCCCGGAAAGGGCCTGGAGTGGGTGGGTGAGATCTCTGCTGCCGGTGGCTACACCGATTACGCCGACTCTGTGAAGGGCCGATTCACCATCTCTGCCGACACCTCTAAGAACACCGCCTACCTGCAGATGAACTCTCTGCGAGCCGAGGACACCGCTGTGTACTACTGTGCACGAGAAAGTAGAGTTTCCTTCGAAGCCGCCATGGACTACTGGGGCCAGGGCACCCTGGTGACCGTGTCCTCT

SEQ ID NO: 23 - protein, 451 aa, synthetic construct
  source 1..451: note = Synthesized
EVQLVESGGGLVQPGGSLRLSCAASGFTLSGDWIHWVRQAPGKGLEWVGEISAAGGYTDYADSVKGRFTISADTSKNTAYLQMNSLRAEDTAVYYCARESRVSFEAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 121 aa, synthetic construct [sequence omitted]

SEQ ID NO: 25 - DNA, 657 bp, synthetic construct
  source 1..657: mol_type = other DNA; note = Synthesized
gatattcaaatgactcaatctccttcttctctttctgcttctgttggtgatcgtgttactattacttgtcgttcttctcaaaatattgttcattctaatggtaatacttatcttgattggtatcaacaaactcctggtaaagctcctaaacttcttatttataaagtttctaatcgtttttctggtgttccttctcgtttttctggttctggttctggtactgattttacttttactatttcttctcttcaacctgaagatattgctacttattattgttttcaatattctcatgttccttggacttttggtcaaggtactaaacttcaaattactcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 26 - DNA, 336 bp, synthetic construct
  source 1..336: mol_type = other DNA; note = Synthesized
gatattcaaatgactcaatctccttcttctctttctgcttctgttggtgatcgtgttactattacttgtcgttcttctcaaaatattgttcattctaatggtaatacttatcttgattggtatcaacaaactcctggtaaagctcctaaacttcttatttataaagtttctaatcgtttttctggtgttccttctcgtttttctggttctggttctggtactgattttacttttactatttcttctcttcaacctgaagatattgctacttattattgttttcaatattctcatgttccttggacttttggtcaaggtactaaacttcaaattact

SEQ ID NO: 27 - protein, 219 aa, synthetic construct
  source 1..219: note = Synthesized
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 112 aa, synthetic construct [sequence omitted]

SEQ ID NO: 29 - DNA, 1359 bp, synthetic construct
  source 1..1359: mol_type = other DNA; note = Synthesized
caagttcaacttcaacaatctggtgctgaagttaaaaaacctggttcttctgttaaagtttcttgtaaagcctctggttatacttttactaattattatatttattgggttcgtcaagctcctggtcaaggtcttgaatggattggtggtattaatcctacttctggtggttctaattttaatgaaaaatttaaaactcgtgttactattactgttgatgaatctacgaacactgcttatatggaactttcttctcttcgttctgaagatactgctttttatttttgtgcgcgtcaaggtctttggtttgattctgatggtcgtggttttgatttttggggtcaaggttccactgttactgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 30 - DNA, 369 bp, synthetic construct
  source 1..369: mol_type = other DNA; note = Synthesized
caagttcaacttcaacaatctggtgctgaagttaaaaaacctggttcttctgttaaagtttcttgtaaagcctctggttatacttttactaattattatatttattgggttcgtcaagctcctggtcaaggtcttgaatggattggtggtattaatcctacttctggtggttctaattttaatgaaaaatttaaaactcgtgttactattactgttgatgaatctacgaacactgcttatatggaactttcttctcttcgttctgaagatactgctttttatttttgtgcgcgtcaaggtctttggtttgattctgatggtcgtggttttgatttttggggtcaaggttccactgttactgtctcgagc

SEQ ID NO: 31 - protein, 453 aa, synthetic construct
  source 1..453: note = Synthesized
QVQLQQSGAEVKKPGSSVKVSCKASGYTFTNYYIYWVRQAPGQGLEWIGGINPTSGGSNFNEKFKTRVTITVDESTNTAYMELSSLRSEDTAFYFCARQGLWFDSDGRGFDFWGQGSTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 123 aa, synthetic construct
  source 1..123: note = Synthesized
QVQLQQSGAEVKKPGSSVKVSCKASGYTFTNYYIYWVRQAPGQGLEWIGGINPTSGGSNFNEKFKTRVTITVDESTNTAYMELSSLRSEDTAFYFCARQGLWFDSDGRGFDFWGQGSTVTVSS

SEQ ID NO: 33 - DNA, 657 bp, synthetic construct; identical to SEQ ID NO: 25
  source 1..657: mol_type = other DNA; note = Synthesized

SEQ ID NO: 34 - DNA, 336 bp, synthetic construct; identical to SEQ ID NO: 26
  source 1..336: mol_type = other DNA; note = Synthesized

SEQ ID NO: 35 - protein, 218 aa, synthetic construct; identical to SEQ ID NO: 3
  source 1..218: note = Synthesized

SEQ ID NO: 36 - protein, 111 aa, synthetic construct
  source 1..111: note = Synthesized
QSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKLTVL

SEQ ID NO: 37 - DNA, 1356 bp, synthetic construct
  source 1..1356: mol_type = other DNA; note = Synthesized
caggtgcagctgcagcagagcggcgccgaggtgaagaagcccggcagcagcgtgaaggtgagctgcaaggccagcggctacaccttcaccaactactacatctactgggtgcggcaggcccccggccagggcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 38 - DNA, 369 bp, synthetic construct
  source 1..369: mol_type = other DNA; note = Synthesized
caggtgcagctgcagcagagcggcgccgaggtgaagaagcccggcagcagcgtgaaggtgagctgcaaggccagcggctacaccttcaccaactactacatctactgggtgcggcaggcccccggccagggcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagc

SEQ ID NO: 39 - protein, 452 aa, synthetic construct
  source 1..452: note = Synthesized
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 123 aa, synthetic construct [sequence omitted]

SEQ ID NO: 41 - DNA, 642 bp, synthetic construct
  source 1..642: mol_type = other DNA; note = Synthesized
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 42 - DNA, 321 bp, synthetic construct
  source 1..321: mol_type = other DNA; note = Synthesized
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaa

SEQ ID NO: 43 - protein, 214 aa, synthetic construct
  source 1..214: note = Synthesized
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 107 aa, synthetic construct
  source 1..107: note = Synthesized
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 45 - DNA, 1347 bp, synthetic construct
  source 1..1347: mol_type = other DNA; note = Synthesized
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 46 - DNA, 357 bp, synthetic construct
  source 1..357: mol_type = other DNA; note = Synthesized
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagc

SEQ ID NO: 47 - protein, 449 aa, synthetic construct
  source 1..449: note = Synthesized
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 119 aa, synthetic construct
  source 1..119: note = Synthesized
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSS

SEQ ID NO: 49 - DNA, 654 bp, synthetic construct; identical to SEQ ID NO: 1
  source 1..654: mol_type = other DNA; note = Synthesized

SEQ ID NO: 50 - DNA, 333 bp, synthetic construct; identical to SEQ ID NO: 2
  source 1..333: mol_type = other DNA; note = Synthesized

SEQ ID NO: 51 - protein, 218 aa, synthetic construct; identical to SEQ ID NO: 3
  source 1..218: note = Synthesized

SEQ ID NO: 52 - protein, 111 aa, synthetic construct; identical to SEQ ID NO: 36
  source 1..111: note = Synthesized

SEQ ID NO: 53 - DNA, 2100 bp, synthetic construct
  source 1..2100: mol_type = other DNA; note = Synthesized
caggtgcagctgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaggcggtggaggatccggcggtggtggatcacaggtgcagctgcaggagtcgggcccaggactggtgaagccttcggagaccctgtccctcacctgcactgtctctggtggctccgtcagcagtggtgattactactggacctggatacggcagtccccagggaagggactggagtggattggacacatctattacagtgggaacaccaattataacccctccctcaagagccgactcaccatatcaattgacacgtccaagactcagttctccctgaagctgagttctgtgaccgctgcggacacggccatttattactgtgtgcgagatcgagtgactggtgcttttgatatctggggccaagggacaatggtcaccgtctcgagcggtggaggcggttcaggcggaggtggttccggcggtggcggctccgacatccagatgacccagtctccatcctccctgtctgcatctgtaggagacagagtcaccatcacttgccaggcgagtcaggacatcagcaactatttaaattggtatcagcagaaaccagggaaagcccctaaactcctgatctacgatgcatccaatttggaaacaggggtcccatcaaggttcagtggaagtggatctgggacagattttactttcaccatcagcagcctgcagcctgaagatattgcaacatatttctgtcaacactttgatcatctcccgctcgctttcggcggagggaccaaggtggaaattaaacgt

SEQ ID NO: 54 - DNA, 354 bp, synthetic construct; identical to SEQ ID NO: 6
  source 1..354: mol_type = other DNA; note = Synthesized

SEQ ID NO: 55 - DNA, 726 bp, synthetic construct
  source 1..726: mol_type = other DNA; note = Synthesized
caggtgcagctgcaggagtcgggcccaggactggtgaagccttcggagaccctgtccctcacctgcactgtctctggtggctccgtcagcagtggtgattactactggacctggatacggcagtccccagggaagggactggagtggattggacacatctattacagtgggaacaccaattataacccctccctcaagagccgactcaccatatcaattgacacgtccaagactcagttctccctgaagctgagttctgtgaccgctgcggacacggccatttattactgtgtgcgagatcgagtgactggtgcttttgatatctggggccaagggacaatggtcaccgtctcgagcggtggaggcggttcaggcggaggtggttccggcggtggcggctccgacatccagatgacccagtctccatcctccctgtctgcatctgtaggagacagagtcaccatcacttgccaggcgagtcaggacatcagcaactatttaaattggtatcagcagaaaccagggaaagcccctaaactcctgatctacgatgcatccaatttggaaacaggggtcccatcaaggttcagtggaagtggatctgggacagattttactttcaccatcagcagcctgcagcctgaagatattgcaacatatttctgtcaacactttgatcatctcccgctcgctttcggcggagggaccaaggtggaaattaaacgt

SEQ ID NO: 56 - protein, 700 aa, synthetic construct
  source 1..700: note = Synthesized
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKR

SEQ ID NO: 57 - protein, 118 aa, synthetic construct; identical to SEQ ID NO: 8
  source 1..118: note = Synthesized

SEQ ID NO: 58 - protein, 242 aa, synthetic construct
  source 1..242: note = Synthesized
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKR

SEQ ID NO: 59 - DNA, 642 bp, synthetic construct
  source 1..642: mol_type = other DNA; note = Synthesized
gacatccagatgacccagtctccatcctccctgtctgcatctgtaggagacagagtcaccatcacttgccaggcgagtcaggacatcagcaactatttaaattggtatcagcagaaaccagggaaagcccctaaactcctgatctacgatgcatccaatttggaaacaggggtcccatcaaggttcagtggaagtggatctgggacagattttactttcaccatcagcagcctgcagcctgaagatattgcaacatatttctgtcaacactttgatcatctcccgctcgctttcggcggagggaccaaggtggaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 60 - DNA, 321 bp, synthetic construct; identical to SEQ ID NO: 10
  source 1..321: mol_type = other DNA; note = Synthesized

SEQ ID NO: 61 - protein, 214 aa, synthetic construct; identical to SEQ ID NO: 11
  source 1..214: note = Synthesized

SEQ ID NO: 62 - protein, 107 aa, synthetic construct [sequence omitted]

SEQ ID NO: 63 - DNA, 2109 bp, synthetic construct
  source 1..2109: mol_type = other DNA; note = Synthesized
caggtgcagctgcaggagtcgggcccaggactggtgaagccttcggagaccctgtccctcacctgcactgtctctggtggctccgtcagcagtggtgattactactggacctggatccggcagtccccagggaagggactggagtggattggacacatctattacagtgggaacaccaattataacccctccctcaagagccgactcaccatatcaattgacacgtccaagactcagttctccctgaagctgagttctgtgaccgctgcggacacggccatttattactgtgtgcgagatcgagtgactggtgcttttgatatctggggccaagggacaatggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaggcggtggaggatccggcggtggtggatcacaggtgcagctgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtccta

SEQ ID NO: 64 - DNA, 357 bp, synthetic construct; identical to SEQ ID NO: 14
  source 1..357: mol_type = other DNA; note = Synthesized

SEQ ID NO: 65 - DNA, 732 bp, synthetic construct
  source 1..732: mol_type = other DNA; note = Synthesized
caggtgcagctgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtccta

SEQ ID NO: 66 - protein, 703 aa, synthetic construct
  source 1..703: note = Synthesized
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 67 - protein, 119 aa, synthetic construct [sequence omitted]

SEQ ID NO: 68 - protein, 243 aa, synthetic construct
  source 1..243: note = Synthesized
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTV

SEQ ID NO: 69 - DNA, 654 bp, synthetic construct; identical to SEQ ID NO: 1
  source 1..654: mol_type = other DNA; note = Synthesized

SEQ ID NO: 70 - DNA, 333 bp, synthetic construct; identical to SEQ ID NO: 2
  source 1..333: mol_type = other DNA; note = Synthesized

SEQ ID NO: 71 - protein, 218 aa, synthetic construct; identical to SEQ ID NO: 3
  source 1..218: note = Synthesized

SEQ ID NO: 72 - protein, 111 aa, synthetic construct; identical to SEQ ID NO: 36
  source 1..111: note = Synthesized

SEQ ID NO: 73 - DNA, 2127 bp, synthetic construct
  source 1..2127: mol_type = other DNA; note = Synthesized
caggtgcagctgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaggcggtggaggatccggcggtggtggatcacaagttcaacttcaacaatctggtgctgaagttaaaaaacctggttcttctgttaaagtttcttgtaaagcctctggttatacttttactaattattatatttattgggttcgtcaagctcctggtcaaggtcttgaatggattggtggtattaatcctacttctggtggttctaattttaatgaaaaatttaaaactcgtgttactattactgttgatgaatctacgaacactgcttatatggaactttcttctcttcgttctgaagatactgctttttatttttgtgcgcgtcaaggtctttggtttgattctgatggtcgtggttttgatttttggggtcaaggttccactgttactgtctcgagcggtggaggcggttcaggcggaggtggttccggcggtggcggctccgatattcaaatgactcaatctccttcttctctttctgcttctgttggtgatcgtgttactattacttgtcgttcttctcaaaatattgttcattctaatggtaatacttatcttgattggtatcaacaaactcctggtaaagctcctaaacttcttatttataaagtttctaatcgtttttctggtgttccttctcgtttttctggttctggttctggtactgattttacttttactatttcttctcttcaacctgaagatattgctacttattattgttttcaatattctcatgttccttggacttttggtcaaggtactaaacttcaaattactcgt

SEQ ID NO: 74 - DNA, 354 bp, synthetic construct; identical to SEQ ID NO: 6
  source 1..354: mol_type = other DNA; note = Synthesized

SEQ ID NO: 75 - DNA, 753 bp, synthetic construct
  source 1..753: mol_type = other DNA; note = Synthesized
caagttcaacttcaacaatctggtgctgaagttaaaaaacctggttcttctgttaaagtttcttgtaaagcctctggttatacttttactaattattatatttattgggttcgtcaagctcctggtcaaggtcttgaatggattggtggtattaatcctacttctggtggttctaattttaatgaaaaatttaaaactcgtgttactattactgttgatgaatctacgaacactgcttatatggaactttcttctcttcgttctgaagatactgctttttatttttgtgcgcgtcaaggtctttggtttgattctgatggtcgtggttttgatttttggggtcaaggttccactgttactgtctcgagcggtggaggcggttcaggcggaggtggttccggcggtggcggctccgatattcaaatgactcaatctccttcttctctttctgcttctgttggtgatcgtgttactattacttgtcgttcttctcaaaatattgttcattctaatggtaatacttatcttgattggtatcaacaaactcctggtaaagctcctaaacttcttatttataaagtttctaatcgtttttctggtgttccttctcgtttttctggttctggttctggtactgattttacttttactatttcttctcttcaacctgaagatattgctacttattattgttttcaatattctcatgttccttggacttttggtcaaggtactaaacttcaaattactcgt

SEQ ID NO: 76 - protein, 709 aa, synthetic construct
  source 1..709: note = Synthesized
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLQQSGAEVKKPGSSVKVSCKASGYTFTNYYIYWVRQAPGQGLEWIGGINPTSGGSNFNEKFKTRVTITVDESTNTAYMELSSLRSEDTAFYFCARQGLWFDSDGRGFDFWGQGSTVTVSSGGGGSGGGGSGGGGSDIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITR

SEQ ID NO: 77 - protein, 118 aa, synthetic construct; identical to SEQ ID NO: 8
  source 1..118: note = Synthesized

SEQ ID NO: 78 - protein, 251 aa, synthetic construct
  source 1..251: note = Synthesized
QVQLQQSGAEVKKPGSSVKVSCKASGYTFTNYYIYWVRQAPGQGLEWIGGINPTSGGSNFNEKFKTRVTITVDESTNTAYMELSSLRSEDTAFYFCARQGLWFDSDGRGFDFWGQGSTVTVSSGGGGSGGGGSGGGGSDIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITR

SEQ ID NO: 79 - DNA, 657 bp, synthetic construct; identical to SEQ ID NO: 25
  source 1..657: mol_type = other DNA; note = Synthesized

SEQ ID NO: 80 - DNA, 336 bp, synthetic construct; identical to SEQ ID NO: 26
  source 1..336: mol_type = other DNA; note = Synthesized

SEQ ID NO: 81 - protein, 219 aa, synthetic construct; identical to SEQ ID NO: 27
  source 1..219: note = Synthesized

SEQ ID NO: 82 - protein, 112 aa, synthetic construct [sequence omitted]

SEQ ID NO: 83 - DNA, 2121 bp, synthetic construct
  source 1..2121: mol_type = other DNA; note = Synthesized
caagttcaacttcaacaatctggtgctgaagttaaaaaacctggttcttctgttaaagtttcttgtaaagcctctggttatacttttactaattattatatttattgggttcgtcaagctcctggtcaaggtcttgaatggattggtggtattaatcctacttctggtggttctaattttaatgaaaaatttaaaactcgtgttactattactgttgatgaatctacgaacactgcttatatggaactttcttctcttcgttctgaagatactgctttttatttttgtgcgcgtcaaggtctttggtttgattctgatggtcgtggttttgatttttggggtcaaggttccactgttactgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaggcggtggaggatccggcggtggtggatcacaggtgcagctgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtccta

SEQ ID NO: 84 - DNA, 369 bp, synthetic construct; identical to SEQ ID NO: 30
  source 1..369: mol_type = other DNA; note = Synthesized

SEQ ID NO: 85 - DNA, 732 bp, synthetic construct; identical to SEQ ID NO: 65
  source 1..732: mol_type = other DNA; note = Synthesized

SEQ ID NO: 86 - protein, 707 aa, synthetic construct
  source 1..707: note = Synthesized
QVQLQQSGAEVKKPGSSVKVSCKASGYTFTNYYIYWVRQAPGQGLEWIGGINPTSGGSNFNEKFKTRVTITVDESTNTAYMELSSLRSEDTAFYFCARQGLWFDSDGRGFDFWGQGSTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 87 - protein, 123 aa, synthetic construct; identical to SEQ ID NO: 32
  source 1..123: note = Synthesized

SEQ ID NO: 88 - protein, 244 aa, synthetic construct
  source 1..244: note = Synthesized
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 89 - DNA, 657 bp, synthetic construct; identical to SEQ ID NO: 25
  source 1..657: mol_type = other DNA; note = Synthesized

SEQ ID NO: 90 - DNA, 336 bp, synthetic construct; identical to SEQ ID NO: 26
  source 1..336: mol_type = other DNA; note = Synthesized

SEQ ID NO: 91 - DNA, 336 bp, synthetic construct
  source 1..336: mol_type = other DNA; note = Synthesized
gacatccagatgacccagtccccttccagcctgtccgcttccgtgggcgacagggtgaccatcacctgcaggtcctcccagaacatcgtgcactccaacggcaatacctacctggactggtaccagcagacccccggcaaggcccctaagctgctgatctacaaggtgagcaaccggttcagcggcgtgccttcccggttttccggatccggctccggcacagacttcaccttcaccatctcctccctgcaacccgaggacatcgccacctactactgcttccagtactcccatgtgccctggaccttcggccagggcaccaagctgcagatcacc

SEQ ID NO: 92 - protein, 218 aa, synthetic construct; identical to SEQ ID NO: 3
  source 1..218: note = Synthesized

SEQ ID NO: 93 - protein, 111 aa, synthetic construct; identical to SEQ ID NO: 36
  source 1..111: note = Synthesized

SEQ ID NO: 94 - DNA, 2118 bp, synthetic construct
  source 1..2118: mol_type = other DNA; note = Synthesized
caggtgcagctgcagcagagcggcgccgaggtgaagaagcccggcagcagcgtgaaggtgagctgcaaggccagcggctacaccttcaccaactactacatctactgggtgcggcaggcccccggccagggcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcacaggtgcaattgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtccta

SEQ ID NO: 95 - DNA, 369 bp, synthetic construct; identical to SEQ ID NO: 38
  source 1..369: mol_type = other DNA; note = Synthesized

SEQ ID NO: 96 - DNA, 369 bp, synthetic construct
  source 1..369: mol_type = other DNA; note = Synthesized
caggtgcagctgcagcaatccggcgccgaggtgaagaagcctggctccagcgtgaaggtgtcctgcaaggcctccggctacaccttcaccaactactacatctactgggtgaggcaggctcctggccagggactggagtggatcggcggcatcaaccctacctccggcggctccaacttcaacgagaagttcaagacccgggtgaccatcaccgccgatgagagctccaccaccgcctacatggagctgtcctccctgaggtccgaggacaccgccttttacttctgcaccaggcagggactgtggttcgactccgacggccggggcttcgatttttggggccagggcaccacagtgaccgtgtcctcc

SEQ ID NO: 97 - DNA, 732 bp, synthetic construct
  source 1..732: mol_type = other DNA; note = Synthesized
caggtgcaattgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtccta

SEQ ID NO: 98 - protein, 706 aa, synthetic construct
  source 1..706: note = Synthesized
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 99 - protein, 123 aa, synthetic construct [sequence omitted]

SEQ ID NO: 100 - protein, 244 aa, synthetic construct; identical to SEQ ID NO: 88
  source 1..244: note = Synthesized

SEQ ID NO: 101 - DNA, 654 bp, synthetic construct; identical to SEQ ID NO: 1
  source 1..654: mol_type = other DNA; note = Synthesized

SEQ ID NO: 102 - DNA, 333 bp, synthetic construct; identical to SEQ ID NO: 2
  source 1..333: mol_type = other DNA; note = Synthesized

SEQ ID NO: 103 - protein, 218 aa, synthetic construct; identical to SEQ ID NO: 3
  source 1..218: note = Synthesized

SEQ ID NO: 104 - protein, 111 aa, synthetic construct; identical to SEQ ID NO: 36
  source 1..111: note = Synthesized

SEQ ID NO: 105 - DNA, 2100 bp, synthetic construct
  source 1..2100: mol_type = other DNA; note = Synthesized
caggtgcagctgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaggcggtggaggatccggcggtggtggatcacaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcggtggaggcggttcaggcggaggtggttccggcggtggcggctccgacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaacgt

SEQ ID NO: 106 - DNA, 354 bp, synthetic construct; identical to SEQ ID NO: 6
  source 1..354: mol_type = other DNA; note = Synthesized

SEQ ID NO: 107 - DNA, 726 bp, synthetic construct
  source 1..726: mol_type = other DNA; note = Synthesized
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcggtggaggcggttcaggcggaggtggttccggcggtggcggctccgacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaacgt

SEQ ID NO: 108 - protein, 700 aa, synthetic construct
  source 1..700: note = Synthesized
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKR

SEQ ID NO: 109 - protein, 118 aa, synthetic construct; identical to SEQ ID NO: 8
  source 1..118: note = Synthesized

SEQ ID NO: 110 - protein, 242 aa, synthetic construct
  source 1..242: note = Synthesized
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKR

SEQ ID NO: 111 - DNA, 642 bp, synthetic construct; identical to SEQ ID NO: 41
  source 1..642: mol_type = other DNA; note = Synthesized

SEQ ID NO: 112 - DNA, 321 bp, synthetic construct; identical to SEQ ID NO: 42
  source 1..321: mol_type = other DNA; note = Synthesized

SEQ ID NO: 113 - protein, 214 aa, synthetic construct; identical to SEQ ID NO: 43
  source 1..214: note = Synthesized

SEQ ID NO: 114 - protein, 107 aa, synthetic construct; identical to SEQ ID NO: 44
  source 1..107: note = Synthesized

SEQ ID NO: 115 - DNA, 2109 bp, synthetic construct
  source 1..2109: mol_type = other DNA; note = Synthesized
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaggcggtggaggatccggcggtggtggatcacaggtgcagctgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtccta

SEQ ID NO: 116 - DNA, 357 bp, synthetic construct; identical to SEQ ID NO: 46
  source 1..357: mol_type = other DNA; note = Synthesized

SEQ ID NO: 117 - DNA, 732 bp, synthetic construct; identical to SEQ ID NO: 65
  source 1..732: mol_type = other DNA; note = Synthesized

SEQ ID NO: 118 - protein, 703 aa, synthetic construct
  source 1..703: note = Synthesized
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 119 - protein, 119 aa, synthetic construct; identical to SEQ ID NO: 48
  source 1..119: note = Synthesized

SEQ ID NO: 120 - protein, 244 aa, synthetic construct; identical to SEQ ID NO: 88
  source 1..244: note = Synthesized

SEQ ID NO: 121 - DNA, 642 bp, synthetic construct; identical to SEQ ID NO: 41
  source 1..642: mol_type = other DNA; note = Synthesized

SEQ ID NO: 122 - DNA, 321 bp, synthetic construct; identical to SEQ ID NO: 42
  source 1..321: mol_type = other DNA; note = Synthesized

SEQ ID NO: 123 - protein, 214 aa, synthetic construct; identical to SEQ ID NO: 43
  source 1..214: note = Synthesized

SEQ ID NO: 124 - protein, 107 aa, synthetic construct; identical to SEQ ID NO: 44
  source 1..107: note = Synthesized

SEQ ID NO: 125 - DNA, 1344 bp, synthetic construct
  source 1..1344: mol_type = other DNA; note = Synthesized
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 126 - DNA, 357 bp, synthetic construct; identical to SEQ ID NO: 46
  source 1..357: mol_type = other DNA; note = Synthesized

SEQ ID NO: 127 - protein, 448 aa, synthetic construct
  source 1..448: note = Synthesized
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 119 aa, synthetic construct; identical to SEQ ID NO: 48
  source 1..119: note = Synthesized

SEQ ID NO: 129 - DNA, 642 bp, synthetic construct; identical to SEQ ID NO: 41
  source 1..642: mol_type = other DNA; note = Synthesized

SEQ ID NO: 130 - DNA, 321 bp, synthetic construct; identical to SEQ ID NO: 42
  source 1..321: mol_type = other DNA; note = Synthesized

SEQ ID NO: 131 - protein, 214 aa, synthetic construct; identical to SEQ ID NO: 43
  source 1..214: note = Synthesized

SEQ ID NO: 132 - protein, 107 aa, synthetic construct; identical to SEQ ID NO: 44
  source 1..107: note = Synthesized

SEQ ID NO: 133 - DNA, 2106 bp, synthetic construct
  source 1..2106: mol_type = other DNA; note = Synthesized
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcacaggtgcaattgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtccta

SEQ ID NO: 134 - DNA, 357 bp, synthetic construct; identical to SEQ ID NO: 46
  source 1..357: mol_type = other DNA; note = Synthesized

SEQ ID NO: 135 - DNA, 732 bp, synthetic construct; identical to SEQ ID NO: 65
  source 1..732: mol_type = other DNA; note = Synthesized

SEQ ID NO: 136 - protein, 702 aa, synthetic construct
  source 1..702: note = Synthesized
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 137 - protein, 119 aa, synthetic construct; identical to SEQ ID NO: 48
  source 1..119: note = Synthesized

SEQ ID NO: 138 - protein, 244 aa, synthetic construct; identical to SEQ ID NO: 88
  source 1..244: note = Synthesized

(19) United States
(12) Patent Application Publication
(10) Pub. No.: US 2026/0078190 A1
(43) Pub. Date: Mar. 19, 2026

(54) Bispecific Tetravalent Antibodies and Methods of Making and Using Thereof

(71) Applicant: SystImmune, Inc., Redmond, WA (US)

(72) Inventors: Zeren Gao, Bellevue, WA (US); Phil Tan, Bellevue, WA (US); Brian R. Kovacevich, Bellevue, WA (US); Blair R. Renshaw, Bellevue, WA (US); Jeffrey B. Adamo, Bellevue, WA (US); Nga Sze Amanda Mak, Bellevue, WA (US); Shi Zhuo, Chengdu (CN); Lan Chen, Chengdu (CN); Yi Zhu, Chengdu (CN)

(73) Assignee: SystImmune, Inc., Redmond, WA (US)

(21) Appl. No.: 19/311,740

(22) Filed: Aug. 27, 2025

Related U.S. Application Data
(63) Continuation of application No. 17/143,204, filed on Jan. 7, 2021, now Pat. No. 12,428,483.
Application No. 17/143,204 is a continuation of application No. 15/119,694, now Pat. No. 10,919,977.
(60) Provisional application No. 62/095,348, filed on Dec. 22, 2014.

(51) Int. Cl. C07K 16/28; A61K 31/337; A61K 33/243; A61K 39/00; A61K 45/06; A61K 47/68; A61P 35/00; C07K 14/705; C07K 16/32

(52) U.S. Cl. CPC C07K 16/2863; A61K 31/337; A61K 33/243; A61K 45/06; A61K 47/6843; A61P 35/00; C07K 14/70521; C07K 14/70532; C07K 14/70578; C07K 16/32; A61K 2039/505

(57) ABSTRACT

A bispecific tetravalent antibody comprising an lgG having a pair of heavy chains and a pair of light chains, and two scFv components being connected to either C or N terminals of the heavy or light chains. The bispecific tetravalent antibody may have a binding specificity for two different members of EGFR family.

### CROSS REFERENCE TO RELATED PATENT APPLICATIONS

This application is a continuation of U.S. patent application Ser. No. 17/143,204, filed Jan. 7, 2021, which is a continuation of U.S. patent application Ser. No. 15/119,694, filed Aug. 17, 2016, now U.S. Pat. No. 10,919,977, which is a U.S. National Stage Application under 35 U.S.C. § 371 of International Patent Application No. PCT/US15/66951, filed Dec. 19, 2015, which claims the benefit of U.S. Provisional Application No. 62/095,348, filed Dec. 22, 2014, the disclosure of each of which is hereby incorporated by reference herein in its entirety.

### SEQUENCE LISTING

This application contains a computer readable Sequence Listing which has been submitted in XML file format via Patent Center, the entire content of which is incorporated by reference herein in its entirety. The Sequence Listing XML file submitted via Patent Center is entitled “14247-824-999_SUB_SEQ_LISTING.xml”, was created on Dec. 3, 2025, and is 459,312 bytes in size.

### TECHNICAL FIELD

The present disclosure generally relates to the technical field of antibody therapeutic agents, and more particularly relates to bispecific tetravalent antibodies against two different members of EGFR family.

### BACKGROUND

Overexpression and/or deregulation of members of the ErbB/HER receptor family such as EGFR, HER2, HER3, HER4 have been shown to play an important role in tumorigenesis in cancers. Mutation and amplification of EGFR or HER2 produce aberrant growth signal which activates downstream signaling pathway contributing to tumorigenesis. Therapeutic antibodies and small-molecule inhibitors directed against EGFR and HER2 have been approved for use in the treatment of cancer (Arteaga et al., Nature Reviews Clinical Oncology 9 16-32, January 2012). Monoclonal antibodies against members of EGFR family such as EGFR and HER2, have demonstrated good clinical responses in colon cancer (Price et al., The Lancet Oncology 15(6), Pages 569-579, May 2014), squamous cell carcinoma of head and neck (Cohen, Cancer Treatment Reviews 40(2014) 567-577), breast and gastric cancers (Arteaga et al., Nature Reviews Clinical Oncology 9 16-32, January 2012). Several therapeutic anti-EGFR antibodies, including cetuximab, panitumumab and nimotuzumab are approved therapeutics for several cancers including metastatic colorectal cancer, head and neck squamous cell carcinoma and glioma (Price and Cohen, Curr Treat Options Oncol. 2012 March; 13(1): 35-46; Bode et al., Expert Opin Biol Ther. 2012 December; 12(12): 1649-59). Unfortunately, many tumors that initially respond to these therapeutic agents eventually progress due to an acquired resistance to the agents (Jackman et al. J Clin Oncol 2010; 28:357-60). Therefore, there exists a need for better cancer therapeutics.

### SUMMARY

The disclosure provides bispecific tetravalent antibodies. The bispecific tetravalent antibodies may include an immunoglobulin G (lgG) moiety with two heavy chains and two light chains, and two scFv moieties being covalently connected to either Cor N terminals of the heavy or light chains. The IgG moiety may have a binding specificity to a first member of EGFR family. The scFv moiety may have a binding specificity to a second member of the EGFR family. The lgG moiety and two scFv moieties are covalently connected to be functional as a bispecific tetravalent antibody. The objectives and advantages of the disclosure will become apparent from the following detailed description of preferred embodiments thereof in connection with the accompanying drawings.

### BRIEF DESCRIPTION OF THE DRAWINGS

Preferred embodiments according to the present disclosure will now be described with reference to the FIGs, in which like reference numerals denote like elements.

FIG. 1 is a diagram showing the domain structures of an example bivalent monospecific immunoglobulin G (lgG) antibody.

FIG. 2 is a diagram showing the domain structure of an example tetravalent bispecific antibody comprising an IgG moiety and two scFv moieties in accordance with one embodiment of the present invention.

FIG. 3 shows the domain structure diagrams of example tetravalent bispecific antibodies 1X1, 1X2, 1X3, 1X4, 1X4.2, 1X5 and 1X6.

FIG. 4 shows the VH domain sequence comparison between SI-1X4 and SI-1X4.2 showing the 5 amino acid differences.

FIGS. 5 and 6 are graphs showing monomeric EGFR binding by BLI.

FIGS. 7, 8, and 9 are graphs showing bispecific ELI binding.

FIG. 10 is a graph showing dimeric EGFR ELISA.

FIG. 11 shows binding kinetics of SI-1C5.2 and SI-1X4.2 with monomeric EGFR as analyzed by Octet.

FIG. 12 shows flow cytometric analysis of SI-1X antibodies binding to A431 cells.

FIG. 13 shows flow cytometric analysis of SI-1X antibodies binding to BxPC3 cells.

FIG. 14 shows flow cytometric analysis of SI-1X4.2 antibody binding to Fadu cells.

FIG. 15 shows flow cytometric analysis of SI-1X4.2 antibody binding toA431 cells.

FIG. 16 shows effect of SI-1X antibodies on A431 cell proliferation.

FIG. 17 shows effect of SI-1X antibodies on A431 cell proliferation.

FIG. 18 shows effect of SI-1X antibodies on BxPC3 cell proliferation.

FIG. 19 shows effect of SI-1X antibodies on BxPC3 cell proliferation.

FIG. 20 shows effect of SI-1X4.2 antibodies on Fadu cell proliferation.

FIG. 21 shows effect of SI-1X4.2 antibodies on A431 cell proliferation.

FIGS. 22A and 22B show ADCC activity of SI-1X antibodies on Fadu cell.

FIG. 22A shows ADCC activity of SI-1X6.4, SI-1C6.2, SI-1C7, SI-1C4, SI-3C9, or the combination of SI-1C6.2+SI-1C7 on Fadu cell. FIG. 22B shows ADCC activity of SI-1X4.2, SI-1C5.2, SI-1C7, SI-1C4, SI-3C9, or the combination of SI-1C5.2+SI-1C7 on Fadu cell.

FIGS. 23A and 23B show ADCC activity of SI-1X antibodies on NCI-H1975 cells. FIG. 23A shows ADCC activity of SI-1X6.4, SI-1C6.2, SI-1C7, SI-1C4, SI-3C9, or the combination of SI-1C6.2+SI-1C7 on NCI-H1975 cells. FIG. 23B shows ADCC activity of SI-1X4.2, SI-1C5.2, SI-1C7, SI-1C4, SI-3C9, or the combination of SI-1C5.2+SI-1C7 on NCI-H1975 cells.

FIG. 24 shows the thermal melting of SI-1X antibodies to demonstrate their stability.

FIGS. 25A-25D show the serum stability of SI-1X antibodies over 7 days period. FIG. 25A shows the serum stability of SI-1C5.2. FIG. 25B shows the serum stability of SI-1X4.2. FIG. 25C shows the serum stability of SI-1C6.2. FIG. 25D shows the serum stability of SI-1X6.4.

FIG. 26 is a graph showing the results of EGFR coated ELISA for the PK study in rat.

FIG. 27 is a graph showing the results of HER3 coated ELISA for the PK study in rat.

FIG. 28 is a graph showing the results of sandwich ELISA for the PK study in rat.

FIG. 29 is a graph showing a plot of mean tumor volume vs days in the mouse xenograft study.

FIG. 30 is a graph showing a plot relative body weight vs weeks in the mouse xenograft study.

### DETAILED DESCRIPTION

The present disclosure This disclosure provides bispecific tetravalent antibodies with superior therapeutic properties or efficacies over the currently known anti-EGFR antibodies. In one embodiment, the antibodies target two members of EGFR family including, without limitation, EFFR and HER3. The bispecific tetravalent antibodies may inhibit both EGFR and HER3 mediated signaling simultaneously therefore overcome resistance in EGFR inhibitor or monoclonal antibody treatment.

It must be noted that as used herein and in the appended claims, the singular forms “a”, “and”, and “the” in Throughout this specification and claims, the word “comprise,” or variations such as “comprises” or “comprising,” will be understood to imply the inclusion of a stated integer or group of integers but not the exclusion of any other integer or group of integers include plural referents unless the context clearly dictates otherwise.

“Antibody fragments” comprise a portion of an intact antibody, preferably the antigen-binding or variable region of the intact antibody. Examples of antibody fragments include Fv, Fab, Fab′, F(ab′)2, Fab′-SH; diabodies; linear antibodies (see U.S. Pat. No. 5,641,870, Example 2; Zapata et al., Protein Eng. 8(10): 1057-1062(1995)); single-chain antibody molecules (e.g. scFv). While in the present description, and throughout the specification, reference is made to antibodies and various properties of antibodies, the same disclosure also applies to functional antibody fragments, e.g. dual action Fab fragments.

In one aspect, the bispecific tetravalent antibodies may include an immunoglobulin G (lgG) moiety with two heavy chains and two light chains, and two scFv moieties being covalently connected to either C or N terminals of the heavy or light chains. The IgG moiety may have a binding specificity to a first member of EGFR family. The scFv moiety may have a binding specificity to a second member of the EGFR family. The IgG moiety may provide stability to the scFv moiety. The bispecific tetravalent antibody may block signalling for both AKT and MAPK/ERK pathways and may mediate antibody dependent cell-mediated cytotoxicity (ADCC) towards cells expressing either one or both antigens. In one embodiment, the bispecific tetravalent antibody is capable of binding both antigens simultaneously. In some embodiments, the bispecific tetravalent antibody provides stronger tumour inhibition in proliferation assays in vitro and in vivo than the mono-specific antibody parental control or combination of the mono-specific antibody parental controls.

In one embodiment, the disclosure provides a bispecific tetravalent antibody having two lgG1 heavy chains, two kappa light chains, and two single chain Fv (scFv) domains. The two lgG1 heavy chains and kappa light chains form an IgG moiety with a binding specificity to a first member of the EGFR family. The two scFv domains have a binding specificity to a second member of the EGFR family, and each scFv domain is connected to the C-terminus of either of the lgG1 heavy chains by a connector with an amino acid sequence (gly-gly-gly-gly-ser)ₙ, also known as (G₄S)ₙ (SEQ ID NO: 329), to provide a lgG1-connector connection. n is an integral of at least 1. For example, n may be 2, 3, 4, 5, 6, 7, 8, 9, 10, or 17. Each scFv domain has a structure order of N terminus-variable heavy-linker-variable light-C terminus. The linker may have an amino acid sequence of (gly-gly-gly-gly-ser)ₘ, also known as (G₄S)ₘ (SEQ ID NO: 332). m may be an integral of at least 2 or at least 3. For example, m may be 3, 4, 5, 6, 11, or 12. In some embodiments, at least one or both of the lgG1 heavy chains are humanized or human. In some embodiments, at least one or both of the kappa light chains are humanized or human.

The EGFR family members may include EGFR, HER2, HER3, a fragment or a derivative thereof. In some embodiments, the first member of the EGFR family may be EGFR, HER2, a fragment or a derivative thereof. In some embodiments, the second member of the EGFR family may be HER3, a fragment or a derivative thereof. In one embodiment, the lgG moiety may have a binding specificity for HER3. In one embodiment, the scFv domains may have a binding specificity for EGFR. In one embodiment, the lgG moiety may have a binding specificity for HER3, and the scFv domains may have a binding specificity for EGFR. In one embodiment, the lgG moiety may have a binding specificity for EGFR. In one embodiment, the scFv domains may have a binding specificity for HER3. In one embodiment, the lgG moiety may a binding specificity for EGFR, and the scFv domains may have a binding specificity for HER3.

In some embodiments, the C terminus of one or both of the lgG1 heavy chains misses an amino acid residue. For example, the lysine reside may be deleted from the C terminus of the lgG1 chain before the connector is fused onto the C-terminus. The deletion of the lysine residue makes the lgG1-connector connection resistant to protease activity.

In some embodiments, one or both of the lgG1 heavy chains contain two mutations in the CH3 domain. For example, the two mutations may be reversion to the common residues in human CH3 domain.

In some embodiments, the lgG1 heavy chains may an amino acid sequences of or with at least 95%, 98% or 99% similarity to SEQ ID NO 7, 15, 23, 31, 39, 47, and 127. In some embodiments, the lgG1 heavy chain, connector, and scFv domain may have an amino acid sequence of or with at least 95%, 98% or 99% similarity to SEQ ID NO 56, 66, 76, 86, 98, 108, 118, and 136. In some embodiments, the kappa light chains may have an amino acid sequence of or with at least 95%, 98% or 99% similarity to SEQ ID NO 3, 11, 19, 27, 35, 43, 51, 61, 71, 81, 92, 103, 113, 123, and 131. In some embodiments, the variable light chain may an amino acid sequence of or with at least 95%, 98% or 99% similarity to SEQ ID NO 4, 12, 20, 28, 36, 44, 52, 62, 72, 82, 93, 104, 114, 124, and 132. In some embodiment, the variable heavy chain may have an amino acid sequence of or with at least 95%, 98% or 99% similarity to SEQ ID NO 8, 16, 24, 32, 40, 48, 57, 67, 77, 87, 99, 109, 119, 128, and 137.

In some embodiments, the lgG moiety has a binding specificity for HER3, and the scFv domains have a binding specificity for EGFR. In one embodiment, the lgG1 heavy chain, connector, and scFv domain have an amino acid sequence of SEQ ID NO 56, and the kappa light chain has an amino acid sequence of SEQ ID NO 51. In one embodiment, the lgG1 heavy chain, connector, and scFv domain have an amino acid sequence of SEQ ID NO 76, and the kappa light chain has an amino acid sequence of SEQ ID NO 71. In one embodiment, the lgG1 heavy chain, connector, and scFv domain have an amino acid sequence of SEQ ID NO 108, and the kappa light chain has an amino acid sequence of SEQ ID NO 103.

In some embodiments, the lgG moiety has a binding specificity for EGFR, and the scFv domains have a binding specificity for HER3. In one embodiment, the lgG1 heavy chain, connector, and scFv domain have an amino acid sequence of SEQ ID NO 66, and the kappa light chain has an amino acid sequence of SEQ ID NO 61. In one embodiment, the lgG1 heavy chain, connector, and scFv domain have an amino acid sequence of SEQ ID NO 86, and the kappa light chain has an amino acid sequence of SEQ ID NO 81. In one embodiment, the lgG1 heavy chain, connector, and scFv domain have an amino acid sequence of SEQ

ID NO 98, and the kappa light chain has an amino acid sequence of SEQ ID NO 92. In one embodiment, the lgG1 heavy chain, connector, and scFv domain have an amino acid sequence of SEQ ID NO 118, and the kappa light chain has an amino acid sequence of SEQ ID NO 113. In one embodiment, the lgG1 heavy chain, connector, and scFv domain have an amino acid sequence of SEQ ID NO 136, and the kappa light chain has an amino acid sequence of SEQ ID NO 131.

The bispecific tetravalent antibodies have the activity of inhibiting cancer cell growth. In certain embodiments, an antibody of the invention has a dissociation constant (Kd) of ≤80 nM, ≤50 nM, ≤30 nM, ≤20 nM, ≤10 nM, or ≤0.1 nM for its target EGRF or HER3. The antibody may bind to both targets simultaneously. In some embodiments, the antibody binds to EGRF and HER3 with a Kd less than 50 nM. In some embodiments, the antibody binds to EGRF and/or HER3 with a Kd less than 40, 30, 25, 20, 19, 18 or 10 nM. In one embodiment, the antibody binds to EGRF with a Kd less than 30 nM and binds to HER3 with a Kd less than 30 nM. In one embodiment, the antibody binds to EGRF with a Kd less than 50 nM and binds to HER3 with a Kd less than 50 nM simultaneously.

In another aspect, the disclosure provides isolated nucleic acids encoding the bispecific tetravalent antibodies or its sub-component disclosed herein.

The sub-component may be the lgG1 heavy chain, the kappa light chain, the variable light chain, or the variable heavy chain.

In a further aspect, the disclosure provides expression vectors having the isolated nucleic acids encoding the bispecific tetravalent antibody or its sub-component disclosed herein. The vectors may be expressible in a host cell. The host cell may be prokaryotic or eukaryotic.

In a further aspect, the disclosure provides host cells having the isolated nucleic acids encoding the bispecific tetravalent antibodies disclosed herein or the expression vectors including such nucleic acid sequences.

In a further aspect, the disclosure provides methods for producing bispecific tetravalent antibodies. In one embodiment, the method may include culturing the above-described host cells so that the antibody is produced.

In a further aspect, the disclosure provides immunoconjugates including the bispecific tetravalent antibodies described herein and a cytotoxic agent.

In a further aspect, the disclosure provides pharmaceutical compositions.

The pharmaceutical composition may include the bispecific tetravalent antibodies or the immunoconjugates described herein and a pharmaceutically acceptable carrier. In some embodiments, the composition may further include radioisotope, radionuclide, a toxin, a therapeutic agent, a chemotherapeutic agent or a combination thereof.

In a further aspect, the disclosure provides methods of treating a subject with a cancer. In one embodiment, the method includes the step of administering to the subject an effective amount of a bispecific tetravalent antibody described herein. The cancer may include cells expressing at least two members of EGFR family including, for example, EGFR, HER2, HER3, a fragment or a derivative thereof. The cancer may be breast cancer, colorectal cancer, pancreatic cancer, head and neck cancer, melanoma, ovarian cancer, prostate cancer, and non-small lung cell cancer, glioma, esophageal cancer, nasopharyngeal cancer, anal cancer, rectal cancer, gastric cancer, bladder cancer, cervical cancer and brain cancer.

In one embodiment, the method may further include co-administering an effective amount of a therapeutic agent. The therapeutic agent may be, for example, an antibody, a chemotherapy agent, a cytotoxic agent, an enzyme, or a combination thereof. In some embodiments, the therapeutic agent may be an anti-estrogen agent, a receptor tyrosine inhibitor, or a combination thereof. In some embodiments, the therapeutic agent may be biologics. In one embodiment, the therapeutic agent may be a checkpoint inhibitor. In some embodiments, the therapeutic agent may include PD1, PDL1, CTLA4, 4-1BB, OX40, GITR, TIM3, LAG3, TIGIT, CD40, CD27, HVEM, BTLA, VISTA, B7H4, a derivative, a conjugate, or a fragment thereof. In some embodiments, the therapeutic agent may be capecitabine, cisplatin, trastuzumab, fulvestrant, tamoxifen, letrozole, exemestane, anastrozole, aminoglutethimide, testolactone, vorozole, formestane, fadrozole, letrozole, erlotinib, lafatinib, dasatinib, gefitinib, imatinib, pazopinib, lapatinib, sunitinib, nilotinib, sorafenib, nab-palitaxel, or a derivative thereof. In some embodiments, the subject in need of such treatment is a human.

In one embodiment, the disclosure provides methods for treating a subject by administering to the subject an effective amount of the bispecific tetravalent antibody to inhibit a biological activity of a HER receptor.

In one embodiment, the disclosure provides solutions having an effective concentration of the bispecific tetravalent antibody. In one embodiment, the solution is blood plasma in a subject.

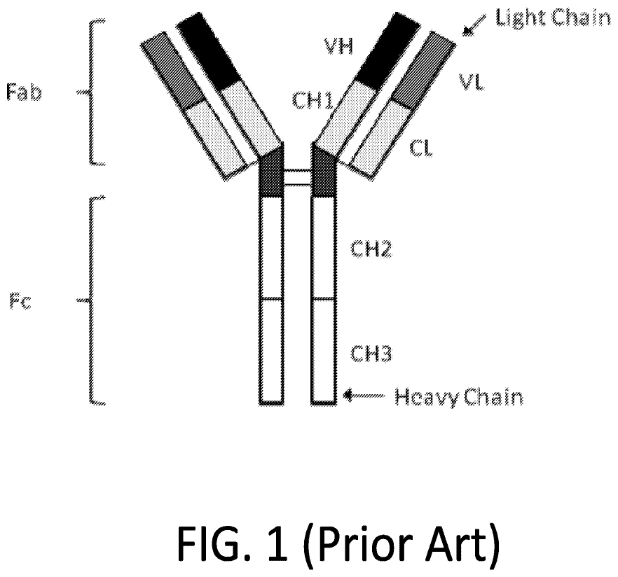

A diagram of the general structure of lgG is shown in FIG. 1.

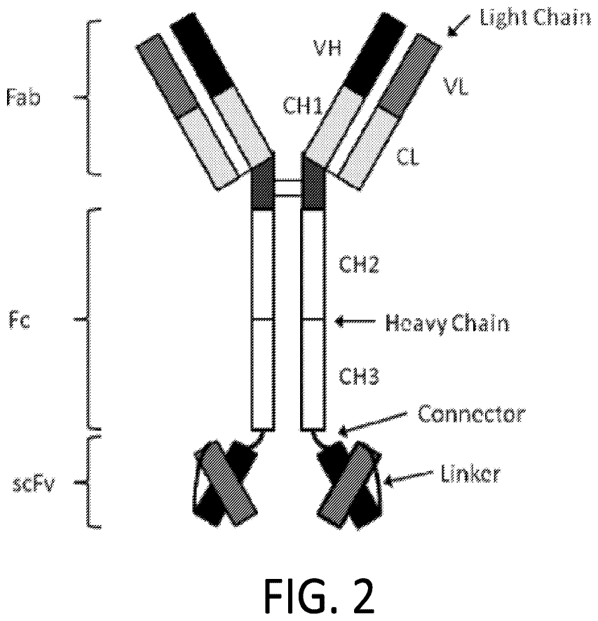

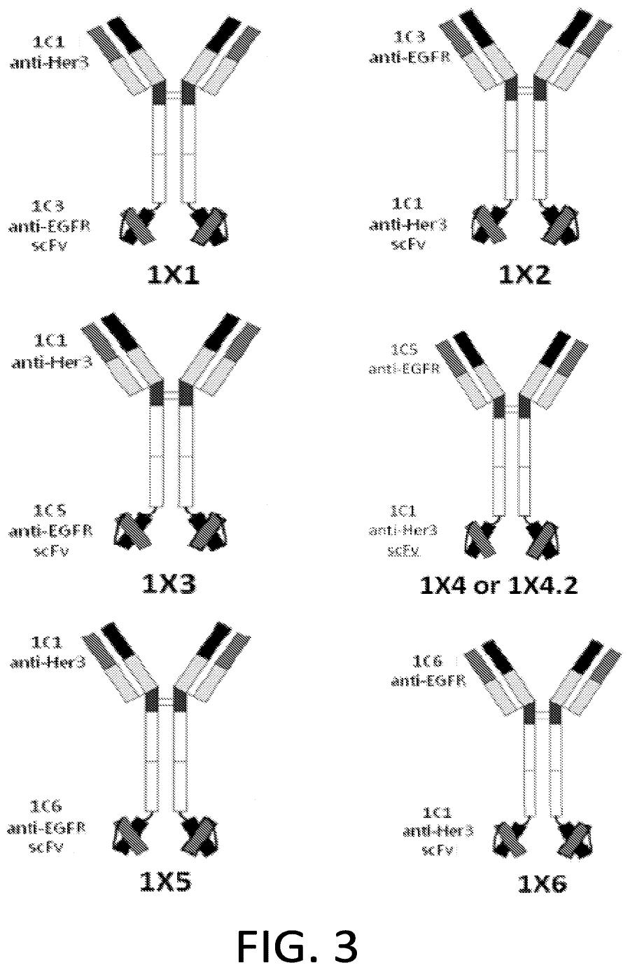

A diagram of the representative structure of the bispecific tetravalent antibodies according to some embodiments is shown in FIG. 2. In this example, the bispecific tetravalent antibody includes two human IgG1 heavy chains, two human kappa light chains, and two single chain Fv (scFv) domains. The two human IgG1 heavy chains and human kappa light chains form an lgG moiety with a binding specificity to one member of the EGFR family, and each of the two scFv domains is connected to the C-terminal residue of either of the human IgG1 heavy chains by a connector with an amino acid sequence of gly-gly-gly-gly-ser-gly-gly-gly-gly-ser ((G₄S)₂) (SEQ ID NO: 330). Each scFv domain is in the order: N terminus-variable heavy-linker-variable light-C terminus. The linker is comprised of amino acid sequence of gly-gly-gly-gly-ser-gly-gly-gly-gly-ser-gly-gly-gly-gly-ser, also known as (G₄S)₃ (SEQ ID NO: 331). For some embodiments of the bispecific tetravalent antibodies, the CH1, CH2, CH3, CL, Connector and Linker amino acid sequences are identical. Each bispecific tetravalent antibody has a bivalent anti-HER3 binding specificity on one end of the antibody and a bivalent anti-EGFR binding specificity on the other end. One pair of anti-HER3 variable heavy chain and variable light chain is designated as 1C1, and four pairs of anti-EGFR variable heavy chains and variable light chains are designated as 1C3, 1C5, 1C5.2, 1C6 and 1C6.4, respectively. The bispecific tetravalent antibodies are designated as 1X1, 1X2, 1X3, 1X4, 1X4.2, 1X5, 1X5.2, 1X6, and 1×6.4

In addition, a control molecule 1C4 (also designated as SI-1C4) was used in some of the studies. 1C4 is a bispecific antibody against EGFR and HER3 built on the two-in-one platform described by Schaefer et. al., 2011 (Schaefer et al., Cancer Cell. 2011 Oct. 18; 20(4): 472-86). IC4 has a similar structure to a monoclonal antibody. The molecule can bind to either EGFR or HER3 on each Fab arm, but cannot engage both targets simultaneously on each Fab arm.

Variable light chain, variable heavy chain and single chain Fv (scFv) DNA fragments were generated by gene synthesis through an outside vendor. Human Gamma-1 heavy chain and human kappa light chain DNA fragments were generated by gene synthesis through an outside vendor. The fragments were assembled together by DNA ligation using restriction sites and cloned into a vector that is designed for transient expression in mammalian cells. The vector contains a strong CMV-derived promoter, and other upstream and downstream elements required for transient expression. The resulting lgG expression plasmids were verified as containing the expected DNA sequences by DNA sequencing.

Transient expression of the antibody constructs was achieved using transfection of suspension-adapted HEK293F cells with linear PEI as described elsewhere (see CSH Protocols; 2008; doi: 10.1101/pdb.prot4977). Antibodies were purified from the resulting transfection supernatants using protein affinity chromatography and size exclusion chromatography if needed. Protein quality is analysed by Superdex 200 column. Protein used for all the assays have a purity of greater than 90%.

The bispecific antibody may be used for the treatment of cancer types with EGFR and HER3 co-expressions, including without limitation colon cancer, head and neck squamous cell carcinoma, lung cancer, glioma, pancreatic cancer, nasopharyngeal cancer and other cancer types.

The bispecific antibody is of tetravalent dual specificity. The example antibody may include an lgG and two scFv, which provides two different binding specificities compared to mono-specific antibody lgG. The IgG component provides stability and improved serum half-life over other bispecific antibodies that used only scFv such as BiTE technology (Lutterbuese et al., Proceedings of the National Academy of Sciences of the United States of America 107.28 (2010): 12605-12610. PMC. Web. 2 Dec. 2014) and others (for example, U.S. Pat. No. 7,332,585B2). It is also capable of mediating ADCC while those without Fc component cannot (for example, U.S. Pat. No. 7,332,585B2). The tetravalent dual specificity nature provides the bispecific antibody a simultaneous binding capability over some other bispecific antibodies, which may only bind one antigen at a time (Schanzer et al, Antimicrob. Agents Chemother. 2011, 55(5): 2369; EP272942A1).

For the convenient of narration, the sequences of or related to the bispecific antibodies are summarized in TABLE 1 herein below.

TABLE 1
Summary of nucleotide and amino acid sequences of or related to the bispecific antibodies.

| | |
|---|---|
| SI-1C1 | |
| SEQUENCES | |
| SEQ ID NO 1 | SI-1C1 LIGHT CHAIN FULL-LENGTH NUCLEOTIDE |
| | SEQUENCE |
| SEQ ID NO 2 | SI-1C1 LIGHT CHAIN VARIABLE LIGHT CHAIN |
| | NUCLEOTIDE SEQUENCE |
| SEQ ID NO 3 | SI-1C1 LIGHT CHAIN FULL-LENGTH AMINO ACID |
| | SEQUENCE. HUMAN KAPPA CONSTANT DOMAIN IS |
| | UNDERLINED |
| SEQ ID NO 4 | SI-1C1 LIGHT CHAIN VARIABLE LIGHT CHAIN |
| | AMINO ACID SEQUENCE. COMPLEMENTARITY |
| | DETERMINING REGIONS ARE UNDERLINED |
| SEQ ID NO 5 | SI-1C1 HEAVY CHAIN FULL-LENGTH NUCLEOTIDE |
| | SEQUENCE |
| SEQ ID NO 6 | SI-1C1 HEAVY CHAIN VARIABLE HEAVY CHAIN |
| | NUCLEOTIDE SEQUENCE |
| SEQ ID NO 7 | SI-1C1 HEAVY CHAIN FULL-LENGTH AMINO ACID |
| | SEQUENCE. HUMAN GAMMA-1 DOMAIN IS |
| | UNDERLINED |
| SEQ ID NO 8 | SI-1C1 HEAVY CHAIN VARIABLE HEAVY CHAIN |
| | AMINO ACID SEQUENCE. COMPLEMENTARITY |
| | DETERMINING REGIONS ARE UNDERLINED |
| SI-1C3 | |
| SEQUENCES | |
| SEQ ID NO 9 | SI-1C3 LIGHT CHAIN FULL-LENGTH NUCLEOTIDE |
| | SEQUENCE |
| SEQ ID NO 10 | SI-1C3 LIGHT CHAIN VARIABLE LIGHT CHAIN |
| | NUCLEOTIDE SEQUENCE |
| SEQ ID NO 11 | SI-1C3 LIGHT CHAIN FULL-LENGTH AMINO ACID |
| | SEQUENCE. HUMAN KAPPA CONSTANT DOMAIN IS |
| | UNDERLINED |
| SEQ ID NO 12 | SI-1C3 LIGHT CHAIN VARIABLE LIGHT CHAIN |
| | AMINO ACID SEQUENCE. COMPLEMENTARITY |
| | DETERMINING REGIONS ARE UNDERLINED |
| SEQ ID NO 13 | SI-1C3 HEAVY CHAIN FULL-LENGTH NUCLEOTIDE |
| | SEQUENCE |
| SEQ ID NO 14 | SI-1C3 HEAVY CHAIN VARIABLE HEAVY CHAIN |
| | NUCLEOTIDE SEQUENCE |
| SEQ ID NO 15 | SI-1C3 HEAVY CHAIN FULL-LENGTH AMINO ACID |
| | SEQUENCE. HUMAN GAMMA-1 DOMAIN IS |
| | UNDERLINED |
| SEQ ID NO 16 | SI-1C3 HEAVY CHAIN VARIABLE HEAVY CHAIN |
| | AMINO ACID SEQUENCE. COMPLEMENTARITY |
| | DETERMINING REGIONS ARE UNDERLINED |
| SI-1C4 | |
| SEQUENCES | |
| SEQ ID NO 17 | SI-1C4 LIGHT CHAIN FULL-LENGTH NUCLEOTIDE |
| | SEQUENCE |
| SEQ ID NO 18 | SI-1C4 LIGHT CHAIN VARIABLE LIGHT CHAIN |
| | NUCLEOTIDE SEQUENCE |
| SEQ ID NO 19 | SI-1C4 LIGHT CHAIN FULL-LENGTH AMINO ACID |
| | SEQUENCE. HUMAN KAPPA CONSTANT DOMAIN IS |
| | UNDERLINED |
| SEQ ID NO 20 | SI-1C4 LIGHT CHAIN VARIABLE LIGHT CHAIN |
| | AMINO ACID SEQUENCE. COMPLEMENTARITY |
| | DETERMINING REGIONS ARE UNDERLINED |
| SEQ ID NO 21 | SI-1C4 HEAVY CHAIN FULL-LENGTH NUCLEOTIDE |
| | SEQUENCE |
| SEQ ID NO 22 | SI-1C4 HEAVY CHAIN VARIABLE HEAVY CHAIN |
| | NUCLEOTIDE SEQUENCE |
| SEQ ID NO 23 | SI-1C4 HEAVY CHAIN FULL-LENGTH AMINO ACID |
| | SEQUENCE. HUMAN GAMMA-1 DOMAIN IS |
| | UNDERLINED |
| SEQ ID NO 24 | SI-1C4 HEAVY CHAIN VARIABLE HEAVY CHAIN |
| | AMINO ACID SEQUENCE. COMPLEMENTARITY |
| | DETERMINING REGIONS ARE UNDERLINED |
| SI-1C5 | |
| SEQUENCES | |
| SEQ ID NO 25 | SI-1C5 LIGHT CHAIN FULL-LENGTH NUCLEOTIDE |
| | SEQUENCE |
| SEQ ID NO 26 | SI-1C5 LIGHT CHAIN VARIABLE LIGHT CHAIN |
| | NUCLEOTIDE SEQUENCE |
| SEQ ID NO 27 | SI-1C5 LIGHT CHAIN FULL-LENGTH AMINO ACID |
| | SEQUENCE. HUMAN KAPPA CONSTANT DOMAIN IS |
| | UNDERLINED |
| SEQ ID NO 28 | SI-1C5 LIGHT CHAIN VARIABLE LIGHT CHAIN |
| | AMINO ACID SEQUENCE. COMPLEMENTARITY |
| | DETERMINING REGIONS ARE UNDERLINED |
| SEQ ID NO 29 | SI-1C5 HEAVY CHAIN FULL-LENGTH NUCLEOTIDE |
| | SEQUENCE |
| SEQ ID NO 30 | SI-1C5 HEAVY CHAIN VARIABLE HEAVY CHAIN |
| | NUCLEOTIDE SEQUENCE |
| SEQ ID NO 31 | SI-1C5 HEAVY CHAIN FULL-LENGTH AMINO ACID |
| | SEQUENCE. HUMAN GAMMA-1 DOMAIN IS |
| | UNDERLINED |
| SEQ ID NO 32 | SI-1C5 HEAVY CHAIN VARIABLE HEAVY CHAIN |
| | AMINO ACID SEQUENCE. COMPLEMENTARITY |
| | DETERMINING REGIONS ARE UNDERLINED |
| SI-1C5.2 | |
| SEQUENCES | |
| SEQ ID NO 33 | SIIC5.2 LIGHT CHAIN FULL-LENGTH NUCLEOTIDE |
| | SEQUENCE |
| SEQ ID NO 34 | SI-1C5.2 LIGHT CHAIN VARIABLE LIGHT CHAIN |
| | NUCLEOTIDE SEQUENCE |
| SEQ ID NO 35 | SI-1C5.2 LIGHT CHAIN FULL-LENGTH AMINO ACID |
| | SEQUENCE. HUMAN KAPPA CONSTANT DOMAIN IS |
| | UNDERLINED |
| SEQ ID NO 36 | SI-1C5.2 LIGHT CHAIN VARIABLE LIGHT CHAIN |
| | AMINO ACID SEQUENCE. COMPLEMENTARITY |
| | DETERMINING REGIONS ARE UNDERLINED |
| SEQ ID NO 37 | SI-1C5.2 HEAVY CHAIN FULL-LENGTH NUCLEOTIDE |
| | SEQUENCE |
| SEQ ID NO 38 | SI-1C5.2 HEAVY CHAIN VARIABLE HEAVY CHAIN |
| | NUCLEOTIDE SEQUENCE |
| SEQ ID NO 39 | SI-1C5.2 HEAVY CHAIN FULL-LENGTH AMINO ACID |
| | SEQUENCE. HUMAN GAMMA-1 DOMAIN IS |
| | UNDERLINED |
| SEQ ID NO 40 | SI-1C5.2 HEAVY CHAIN VARIABLE HEAVY CHAIN |
| | AMINO ACID SEQUENCE. COMPLEMENTARITY |
| | DETERMINING REGIONS ARE UNDERLINED |
| SI-1C6 | |
| SEQUENCES | |
| SEQ ID NO 41 | SI-1C6 LIGHT CHAIN FULL-LENGTH NUCLEOTIDE |
| | SEQUENCE |
| SEQ ID NO 42 | SI-1C6 LIGHT CHAIN VARIABLE LIGHT CHAIN |
| | NUCLEOTIDE SEQUENCE |
| SEQ ID NO 43 | SI-1C6 LIGHT CHAIN FULL-LENGTH AMINO ACID |
| | SEQUENCE. HUMAN KAPPA CONSTANT DOMAIN IS |
| | UNDERLINED |
| SEQ ID NO 44 | SI-1C6 LIGHT CHAIN VARIABLE LIGHT CHAIN |
| | AMINO ACID SEQUENCE. COMPLEMENTARITY |
| | DETERMINING REGIONS ARE UNDERLINED |
| SEQ ID NO 45 | SI-1C6 HEAVY CHAIN FULL-LENGTH NUCLEOTIDE |
| | SEQUENCE |
| SEQ ID NO 46 | SI-1C6 HEAVY CHAIN VARIABLE HEAVY CHAIN |
| | NUCLEOTIDE SEQUENCE |
| SEQ ID NO 47 | SI-1C6 HEAVY CHAIN FULL-LENGTH AMINO ACID |
| | SEQUENCE. HUMAN GAMMA-1 DOMAIN IS |
| | UNDERLINED |
| SEQ ID NO 48 | SI-1C6 HEAVY CHAIN VARIABLE HEAVY CHAIN |
| | AMINO ACID SEQUENCE. COMPLEMENTARITY |
| | DETERMINING REGIONS ARE UNDERLINED |
| SI-1X1 | |
| SEQUENCES | |
| SEQ ID NO 49 | SI1X1 LIGHT CHAIN FULL-LENGTH NUCLEOTIDE |
| | SEQUENCE |
| SEQ ID NO 50 | SI-1X1 LIGHT CHAIN VARIABLE LIGHT CHAIN |
| | NUCLEOTIDE SEQUENCE |
| SEQ ID NO 51 | SI-1X1 LIGHT CHAIN FULL-LENGTH AMINO ACID |
| | SEQUENCE. HUMAN KAPPA CONSTANT DOMAIN IS |
| | UNDERLINED |
| SEQ ID NO 52 | SI-1X1 LIGHT CHAIN VARIABLE LIGHT CHAIN |
| | AMINO ACID SEQUENCE. COMPLEMENTARITY |
| | DETERMINING REGIONS ARE UNDERLINED |
| SEQ ID NO 53 | SI1X1 BISPECIFIC HEAVY CHAIN FULL-LENGTH |
| | NUCLEOTIDE SEQUENCE |
| SEQ ID NO 54 | SI-1X1 BISPECIFIC HEAVY CHAIN VARIABLE |
| | HEAVY CHAIN NUCLEOTIDE SEQUENCE |
| SEQ ID NO 55 | SI-1X1 BISPECIFIC HEAVY CHAIN SCFV |
| | NUCLEOTIDE SEQUENCE |
| SEQ ID NO 56 | SI-1X1 BISPECIFIC HEAVY CHAIN FULL-LENGTH |
| | AMINO ACID SEQUENCE. HUMAN GAMMA-1 |
| | DOMAIN IS UNDERLINED, CONNECTOR IS IN |
| | ITALICS, SCFV IS IN BOLD |
| SEQ ID NO 57 | SI-1X1 BISPECIFIC HEAVY CHAIN VARIABLE |
| | HEAVY CHAIN AMINO ACID SEQUENCE. |
| | COMPLEMENTARITY DETERMINING REGIONS ARE |
| | UNDERLINED |
| SEQ ID NO 58 | SI1X1 BISPECIFIC HEAVY CHAIN SCFV AMINO ACID |
| | SEQUENCE. ORDER: VH-LINKER-VL. |
| | COMPLEMENTARITY DETERMINING REGIONS ARE |
| | UNDERLINED. LINKER IS IN BOLD ITALICS |
| SI-1X2 | |
| SEQUENCES | |
| SEQ ID NO 59 | SI-1X2 LIGHT CHAIN FULL-LENGTH NUCLEOTIDE |
| | SEQUENCE |
| SEQ ID NO 60 | SI-1X2 LIGHT CHAIN VARIABLE LIGHT CHAIN |
| | NUCLEOTIDE SEQUENCE |
| SEQ ID NO 61 | SI-1X2 LIGHT CHAIN FULL-LENGTH AMINO ACID |
| | SEQUENCE. HUMAN KAPPA CONSTANT DOMAIN IS |
| | UNDERLINED |
| SEQ ID NO 62 | SI-1X2 LIGHT CHAIN VARIABLE LIGHT CHAIN |
| | AMINO ACID SEQUENCE. COMPLEMENTARITY |
| | DETERMINING REGIONS ARE UNDERLINED |
| SEQ ID NO 63 | SI-1X2 BISPECIFIC HEAVY CHAIN FULL-LENGTH |
| | NUCLEOTIDE SEQUENCE |
| SEQ ID NO 64 | SI-1X2 BISPECIFIC HEAVY CHAIN VARIABLE |
| | HEAVY CHAIN NUCLEOTIDE SEQUENCE |
| SEQ ID NO 65 | SI-1X2 BISPECIFIC HEAVY CHAIN SCFV |
| | NUCLEOTIDE SEQUENCE |
| SEQ ID NO 66 | SI-1X2 BISPECIFIC HEAVY CHAIN FULL-LENGTH |
| | AMINO ACID SEQUENCE. HUMAN GAMMA-1 |
| | DOMAIN IS UNDERLINED, CONNECTOR IS IN |
| | ITALICS, SCFV IS IN BOLD |
| SEQ ID NO 67 | SI-1X2 BISPECIFIC HEAVY CHAIN VARIABLE |
| | HEAVY CHAIN AMINO ACID SEQUENCE. |
| | COMPLEMENTARITY DETERMINING REGIONS ARE |
| | UNDERLINED |
| SEQ ID NO 68 | SI-1X2 BISPECIFIC HEAVY CHAIN SCFV AMINO |
| | ACID SEQUENCE. ORDER: VH-LINKER-VL. |
| | COMPLEMENTARITY DETERMINING REGIONS ARE |
| | UNDERLINED. LINKER IS IN BOLD ITALICS |
| SI-1X3 | |
| SEQUENCES | |
| SEQ ID NO 69 | SI-1X3 LIGHT CHAIN FULL-LENGTH NUCLEOTIDE |
| | SEQUENCE |
| SEQ ID NO 70 | SI-1X3 LIGHT CHAIN VARIABLE LIGHT CHAIN |
| | NUCLEOTIDE SEQUENCE |
| SEQ ID NO 71 | SI-1X3 LIGHT CHAIN FULL-LENGTH AMINO ACID |
| | SEQUENCE. HUMAN KAPPA CONSTANT DOMAIN IS |
| | UNDERLINED |
| SEQ ID NO 72 | SI-1X3 LIGHT CHAIN VARIABLE LIGHT CHAIN |
| | AMINO ACID SEQUENCE. COMPLEMENTARITY |
| | DETERMINING REGIONS ARE UNDERLINED |
| SEQ ID NO 73 | SI-1X3 BISPECIFIC HEAVY CHAIN FULL-LENGTH |
| | NUCLEOTIDE SEQUENCE |
| SEQ ID NO 74 | SI-1X3 BISPECIFIC HEAVY CHAIN VARIABLE |
| | HEAVY CHAIN NUCLEOTIDE SEQUENCE |
| SEQ ID NO 75 | SI-1X3 BISPECIFIC HEAVY CHAIN SCFV |
| | NUCLEOTIDE SEQUENCE |
| SEQ ID NO 76 | SI-1X3 BISPECIFIC HEAVY CHAIN FULL-LENGTH |
| | AMINO ACID SEQUENCE. HUMAN GAMMA-1 |
| | DOMAIN IS UNDERLINED, CONNECTOR IS IN |
| | ITALICS, SCFV IS IN BOLD |
| SEQ ID NO 77 | SI-1X3 BISPECIFIC HEAVY CHAIN VARIABLE |
| | HEAVY CHAIN AMINO ACID SEQUENCE. |
| | COMPLEMENTARITY DETERMINING REGIONS ARE |
| | UNDERLINED |
| SEQ ID NO 78 | SI-1X3 BISPECIFIC HEAVY CHAIN SCFV AMINO |
| | ACID SEQUENCE. ORDER: VH-LINKER-VL. |
| | COMPLEMENTARITY DETERMINING REGIONS ARE |
| | UNDERLINED. LINKER IS IN BOLD ITALICS |
| SI-1X4 | |
| SEQUENCES | |
| SEQ ID NO 79 | SI-1X4 LIGHT CHAIN FULL-LENGTH NUCLEOTIDE |
| | SEQUENCE |
| SEQ ID NO 80 | SI-1X4 LIGHT CHAIN VARIABLE LIGHT CHAIN |
| | NUCLEOTIDE SEQUENCE |
| SEQ ID NO 81 | SI-1X4 LIGHT CHAIN FULL-LENGTH AMINO ACID |
| | SEQUENCE. HUMAN KAPPA CONSTANT DOMAIN IS |
| | UNDERLINED |
| SEQ ID NO 82 | SI-1X4 LIGHT CHAIN VARIABLE LIGHT CHAIN |
| | AMINO ACID SEQUENCE. COMPLEMENTARITY |
| | DETERMINING REGIONS ARE UNDERLINED |
| SEQ ID NO 83 | SI-1X4 BISPECIFIC HEAVY CHAIN FULL-LENGTH |
| | NUCLEOTIDE SEQUENCE |
| SEQ ID NO 84 | SI-1X4 BISPECIFIC HEAVY CHAIN VARIABLE |
| | HEAVY CHAIN NUCLEOTIDE SEQUENCE |
| SEQ ID NO 85 | SI-1X4 BISPECIFIC HEAVY CHAIN SCFV |
| | NUCLEOTIDE SEQUENCE |
| SEQ ID NO 86 | SI-1X4 BISPECIFIC HEAVY CHAIN FULL-LENGTH |
| | AMINO ACID SEQUENCE. HUMAN GAMMA-1 |
| | DOMAIN IS UNDERLINED, CONNECTOR IS IN |
| | ITALICS, SCFV IS IN BOLD |
| SEQ ID NO 87 | SI-1X4 BISPECIFIC HEAVY CHAIN VARIABLE |
| | HEAVY CHAIN AMINO ACID SEQUENCE. |
| | COMPLEMENTARITY DETERMINING REGIONS ARE |
| | UNDERLINED |
| SEQ ID NO 88 | SI-1X4 BISPECIFIC HEAVY CHAIN SCFV AMINO |
| | ACID SEQUENCE. ORDER: VH-LINKER-VL. |
| | COMPLEMENTARITY DETERMINING REGIONS ARE |
| | UNDERLINED. LINKER IS IN BOLD ITALICS |
| SI-1X4.2 | |
| SEQUENCES | |
| SEQ ID NO 89 | SI-1X4.2 LIGHT CHAIN FULL-LENGTH NUCLEOTIDE |
| | SEQUENCE |
| SEQ ID NO 90 | SI-1X4.2 LIGHT CHAIN VARIABLE LIGHT CHAIN |
| | NUCLEOTIDE SEQUENCE |
| SEQ ID NO 91 | SI-1X4.2 LIGHT CHAIN VARIABLE LIGHT CHAIN |
| | NUCLEOTIDE SEQUENCE CODON OPTIMIZED FOR |
| | CHO EXPRESSION |
| SEQ ID NO 92 | SI-1X4.2 LIGHT CHAIN FULL-LENGTH AMINO ACID |
| | SEQUENCE. HUMAN KAPPA CONSTANT DOMAIN IS |
| | UNDERLINED |
| SEQ ID NO 93 | SI-1X4.2 LIGHT CHAIN VARIABLE LIGHT CHAIN |
| | AMINO ACID SEQUENCE. COMPLEMENTARITY |
| | DETERMINING REGIONS ARE UNDERLINED |
| SEQ ID NO 94 | SI-1X4.2 BISPECIFIC HEAVY CHAIN FULL-LENGTH |
| | NUCLEOTIDE SEQUENCE |
| SEQ ID NO 95 | SI-1X4.2 BISPECIFIC HEAVY CHAIN VARIABLE |
| | HEAVY CHAIN NUCLEOTIDE SEQUENCE |
| SEQ ID NO 96 | SI-1X4.2 BISPECIFIC HEAVY CHAIN VARIABLE |
| | HEAVY CHAIN NUCLEOTIDE SEQUENCE CODON |
| | OPTIMIZED FOR CHO EXPRESSION |
| SEQ ID NO 97 | SI-1X4.2 BISPECIFIC HEAVY CHAIN SCFV |
| | NUCLEOTIDE SEQUENCE |
| SEQ ID NO 98 | SI-1X4.2 BISPECIFIC HEAVY CHAIN FULL-LENGTH |
| | AMINO ACID SEQUENCE. HUMAN GAMMA-1 |
| | DOMAIN IS UNDERLINED, CONNECTOR IS IN |
| | ITALICS, SCFV IS IN BOLD |
| SEQ ID NO 99 | SI-1X4.2 BISPECIFIC HEAVY CHAIN VARIABLE |
| | HEAVY CHAIN AMINO ACID SEQUENCE. |
| | COMPLEMENTARITY DETERMINING REGIONS ARE |
| | UNDERLINED |
| SEQ ID NO 100 | SI-1X4.2 BISPECIFIC HEAVY CHAIN SCFV AMINO |
| | ACID SEQUENCE. ORDER: VH-LINKER-VL. |
| | COMPLEMENTARITY DETERMINING REGIONS ARE |
| | UNDERLINED. LINKER IS IN BOLD ITALICS |
| SI-1X5 | |
| SEQUENCES | |
| SEQ ID NO 101 | SI-1X5 LIGHT CHAIN FULL-LENGTH NUCLEOTIDE |
| | SEQUENCE |
| SEQ ID NO 102 | SI-1X5 LIGHT CHAIN VARIABLE LIGHT CHAIN |
| | NUCLEOTIDE SEQUENCE |
| SEQ ID NO 103 | SI-1X5 LIGHT CHAIN FULL-LENGTH AMINO ACID |
| | SEQUENCE. HUMAN KAPPA CONSTANT DOMAIN IS |
| | UNDERLINED |
| SEQ ID NO 104 | SI-1X5 LIGHT CHAIN VARIABLE LIGHT CHAIN |
| | AMINO ACID SEQUENCE. COMPLEMENTARITY |
| | DETERMINING REGIONS ARE UNDERLINED |
| SEQ ID NO 105 | SI-1X5 BISPECIFIC HEAVY CHAIN FULL-LENGTH |
| | NUCLEOTIDE SEQUENCE |
| SEQ ID NO 106 | SI-1X5 BISPECIFIC HEAVY CHAIN VARIABLE |
| | HEAVY CHAIN NUCLEOTIDE SEQUENCE |
| SEQ ID NO 107 | SI-1X5 BISPECIFIC HEAVY CHAIN SCFV |
| | NUCLEOTIDE SEQUENCE |
| SEQ ID NO 108 | SI-1X5 BISPECIFIC HEAVY CHAIN FULL-LENGTH |
| | AMINO ACID SEQUENCE. HUMAN GAMMA-1 |
| | DOMAIN IS UNDERLINED, CONNECTOR IS IN |
| | ITALICS, SCFV IS IN BOLD |
| SEQ ID NO 109 | SI-1X5 BISPECIFIC HEAVY CHAIN VARIABLE |
| | HEAVY CHAIN AMINO ACID SEQUENCE. |
| | COMPLEMENTARITY DETERMINING REGIONS ARE |
| | UNDERLINED |
| SEQ ID NO 110 | SI-1X5 BISPECIFIC HEAVY CHAIN SCFV AMINO |
| | ACID SEQUENCE. ORDER: VH-LINKER-VL. |
| | COMPLEMENTARITY DETERMINING REGIONS ARE |
| | UNDERLINED. LINKER IS IN BOLD ITALICS |
| SI-1X6 | |
| SEQUENCES | |
| SEQ ID NO 111 | SI-1X6 LIGHT CHAIN FULL-LENGTH NUCLEOTIDE |
| | SEQUENCE |
| SEQ ID NO 112 | SI-1X6 LIGHT CHAIN VARIABLE LIGHT CHAIN |
| | NUCLEOTIDE SEQUENCE |
| SEQ ID NO 113 | SI-1X6 LIGHT CHAIN FULL-LENGTH AMINO ACID |
| | SEQUENCE. HUMAN KAPPA CONSTANT DOMAIN IS |
| | UNDERLINED |
| SEQ ID NO 114 | SI-1X6 LIGHT CHAIN VARIABLE LIGHT CHAIN |
| | AMINO ACID SEQUENCE. COMPLEMENTARITY |
| | DETERMINING REGIONS ARE UNDERLINED |
| SEQ ID NO 115 | SI-1X6 BISPECIFIC HEAVY CHAIN FULL-LENGTH |
| | NUCLEOTIDE SEQUENCE |
| SEQ ID NO 116 | SI-1X6 BISPECIFIC HEAVY CHAIN VARIABLE |
| | HEAVY CHAIN NUCLEOTIDE SEQUENCE |
| SEQ ID NO 117 | SI-1X6 BISPECIFIC HEAVY CHAIN SCFV |
| | NUCLEOTIDE SEQUENCE |
| SEQ ID NO 118 | SI-1X6 BISPECIFIC HEAVY CHAIN FULL-LENGTH |
| | AMINO ACID SEQUENCE. HUMAN GAMMA-1 |
| | DOMAIN IS UNDERLINED, CONNECTOR IS IN |
| | ITALICS, SCFV IS IN BOLD |
| SEQ ID NO 119 | SI-1X6 BISPECIFIC HEAVY CHAIN VARIABLE |
| | HEAVY CHAIN AMINO ACID SEQUENCE. |
| | COMPLEMENTARITY DETERMINING REGIONS ARE |
| | UNDERLINED |
| SEQ ID NO 120 | SI-1X6 BISPECIFIC HEAVY CHAIN SCFV AMINO |
| | ACID SEQUENCE. ORDER: VH-LINKER-VL. |
| | COMPLEMENTARITY DETERMINING REGIONS ARE |
| | UNDERLINED. LINKER IS IN BOLD ITALICS |
| SI-1C6.2 | |
| SEQUENCES | |
| SEQ ID NO 121 | SI-1C6.2 LIGHT CHAIN FULL-LENGTH NUCLEOTIDE |
| | SEQUENCE |
| SEQ ID NO 122 | SI-1C6.2 LIGHT CHAIN VARIABLE LIGHT CHAIN |
| | NUCLEOTIDE SEQUENCE |
| SEQ ID NO 123 | SI-1C6.2 LIGHT CHAIN FULL-LENGTH AMINO ACID |
| | SEQUENCE. HUMAN KAPPA CONSTANT DOMAIN IS |
| | UNDERLINED |
| SEQ ID NO 124 | SI-1C6.2 LIGHT CHAIN VARIABLE LIGHT CHAIN AMINO |
| | ACID SEQUENCE. COMPLEMENTARITY DETERMINING |
| | REGIONS ARE UNDERLINED |
| SEQ ID NO 125 | SI-1C6.2 HEAVY CHAIN FULL-LENGTH NUCLEOTIDE |
| | SEQUENCE |
| SEQ ID NO 126 | SI-1C6.2 HEAVY CHAIN VARIABLE HEAVY CHAIN |
| | NUCLEOTIDE SEQUENCE |
| SEQ ID NO 127 | SI-1C6.2 HEAVY CHAIN FULL-LENGTH AMINO ACID |
| | SEQUENCE. HUMAN GAMMA-1 DOMAIN IS UNDERLINED |
| SEQ ID NO 128 | SI-1C6.2 HEAVY CHAIN VARIABLE HEAVY CHAIN |
| | AMINO ACID SEQUENCE. COMPLEMENTARITY |
| | DETERMINING REGIONS ARE UNDERLINED |
| SI-1X6.4 | |
| SEQUENCES | |
| SEQ ID NO 129 | SI-1X6.4 LIGHT CHAIN FULL-LENGTH NUCLEOTIDE |
| | SEQUENCE |
| SEQ ID NO 130 | SI-1X6.4 LIGHT CHAIN VARIABLE LIGHT CHAIN |
| | NUCLEOTIDE SEQUENCE |
| SEQ ID NO 131 | SI-1X6.4 LIGHT CHAIN FULL-LENGTH AMINO ACID |
| | SEQUENCE. HUMAN KAPPA CONSTANT DOMAIN IS |
| | UNDERLINED |
| SEQ ID NO 132 | SI-1X6.4 LIGHT CHAIN VARIABLE LIGHT CHAIN AMINO |
| | ACID SEQUENCE. COMPLEMENTARITY DETERMINING |
| | REGIONS ARE UNDERLINED |
| SEQ ID NO 133 | SI-1X6.4 BISPECIFIC HEAVY CHAIN FULL-LENGTH |
| | NUCLEOTIDE SEQUENCE |
| SEQ ID NO 134 | SI-1X6.4 BISPECIFIC HEAVY CHAIN VARIABLE HEAVY |
| | CHAIN NUCLEOTIDE SEQUENCE |
| SEQ ID NO 135 | SI-1X6.4 BISPECIFIC HEAVY CHAIN SCFV NUCLEOTIDE |
| | SEQUENCE |
| SEQ ID NO 136 | SI-1X6.4 BISPECIFIC HEAVY CHAIN FULL-LENGTH |
| | AMINO ACID SEQUENCE. HUMAN GAMMA-1 DOMAIN IS |
| | UNDERLINED, CONNECTOR IS IN ITALICS, SCFV IS IN |
| | BOLD |
| SEQ ID NO 137 | SI-1X6.4 BISPECIFIC HEAVY CHAIN VARIABLE HEAVY |
| | CHAIN AMINO ACID SEQUENCE. COMPLEMENTARITY |
| | DETERMINING REGIONS ARE UNDERLINED |
| SEQ ID NO 138 | SI-1X6.4 BISPECIFIC HEAVY CHAIN SCFV AMINO ACID |
| | SEQUENCE. ORDER: VH-LINKER-VL. |
| | COMPLEMENTARITY DETERMINING REGIONS ARE |
| | UNDERLINED. LINKER IS IN BOLD ITALICS |

### EXAMPLES

While The following examples are provided by way of illustration only and not by way of limitation. Those of skill in the art will readily recognize a variety of non-critical parameters that could be changed or modified to yield essentially the same or similar results.

### Example 1: Sequence Differences Between SI-1X4 and SI-1X4.2

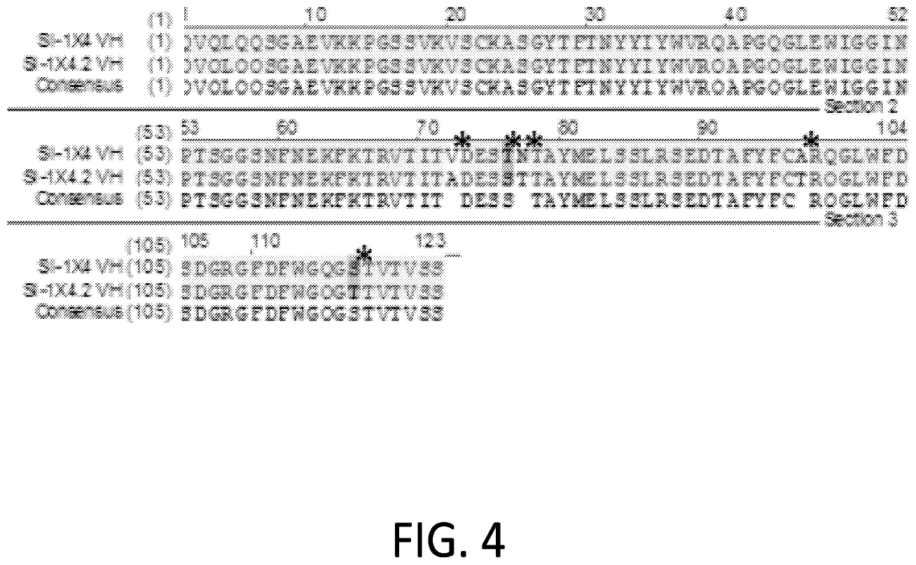

SI-1X4.2 is a modification of SI-1X4 molecule and contained 5 amino acid changes as follows: V71A, T75S, N76S, A93T and S107T using the Kabat 10 numbering system. Some of these changes especially positions 75, 76 and 93 potentially made interaction with antigen even though these are not in the CDR loops and are essential for binding and activity. FIG. 4 shows the 5 amino acid differences between SI-1X4.2 and SI-1X4.

### Example 2: Characterization of Antibodies Against Epidermal Growth Factor Receptor Using BLI

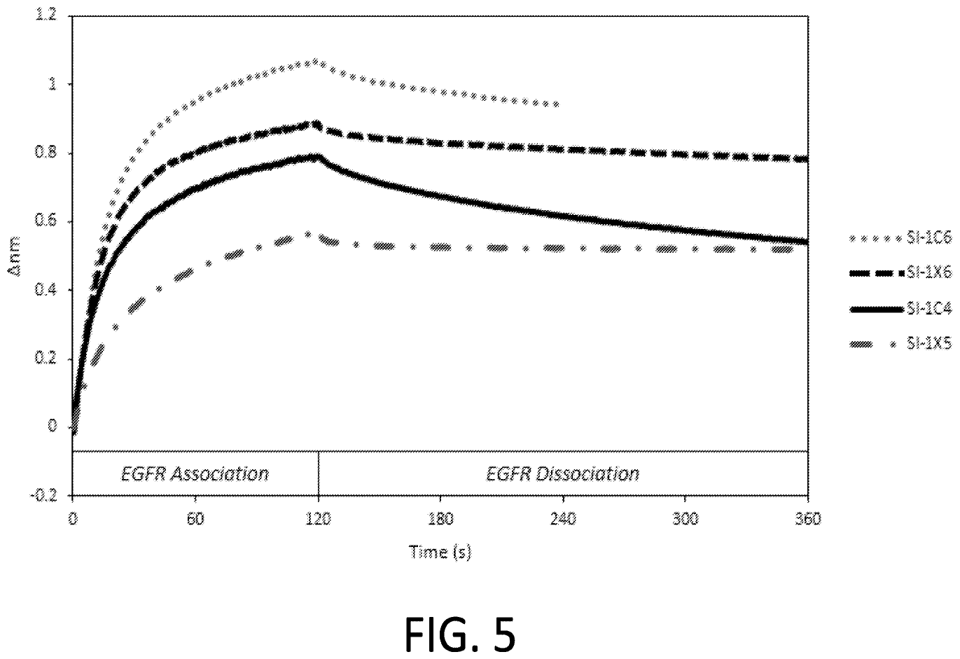

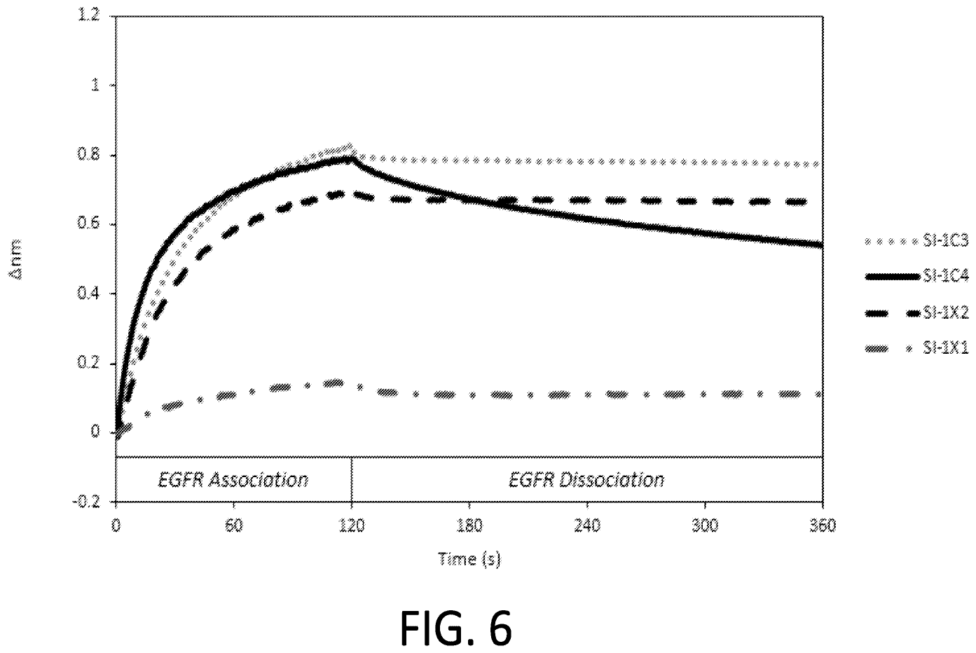

Monomeric EGFR extracellular domain binding was measured in a biolayer interferometry (BLI) binding assay on a BLItz instrument (ForteBio, Inc.). 25 μg/mL of SI-1C3, SI-1C4, SI-1C6, SI-1X1, SI-1X2, SI-1X5, and SI-1X6 were diluted in PBS and captured on anti-huIgG Fc BLItz biosensor tips for 120 seconds. Tips were washed for 30 seconds in PBS and moved to an EGFR (ProSpec Bio, PKA-344) sample for binding at 588 nM. Binding of EGFR ECD to the tips was recorded as biolayer interferometry signals (Anm) over an association time of 120 seconds. Tips were moved to PBS and dissociation was observed for 240 seconds (*SI-1C6 dissociation time of only 120 seconds observed). FIGS. 5 and 6 report data starting at the association step of EGFR to the antibody-loaded biosensor. Each Figure shows comparison to SI-1C4 as a benchmark antibody.

Since SI-1C3 and SI-1X2 share their EGFR binding domain displayed as a Fab, their binding profiles are similar and stronger than the scFv form displayed on SI-1X1 (FIG. 6). Each has a very slow off-rate to EGFR compared to SI-1C4 and is not affected by their on-rate. SI-1X1 may show weaker on-rate binding to EGFR, but stays bound very strongly. The same trend is observed in FIG. 5, where the Fab versions of the EGFR binding domains displayed on SI-1C6 and SI-1X6 bind at a faster rate than their representative scFv displayed on SI-1X5. Having the EGFR binding domain on the Fab side of the bispecifics antibody appears to bind with faster on-rates than the scFv versions, yet exhibit similar off-rates. SI-1X3 and SI-1X4 do not exhibit monomeric EGFR binding in this assay (data not shown) and dimeric EGFR binding is investigated in an ELISA below.

### Example 3: Characterization of Antibodies Against EGFR and Her3 Using BL1

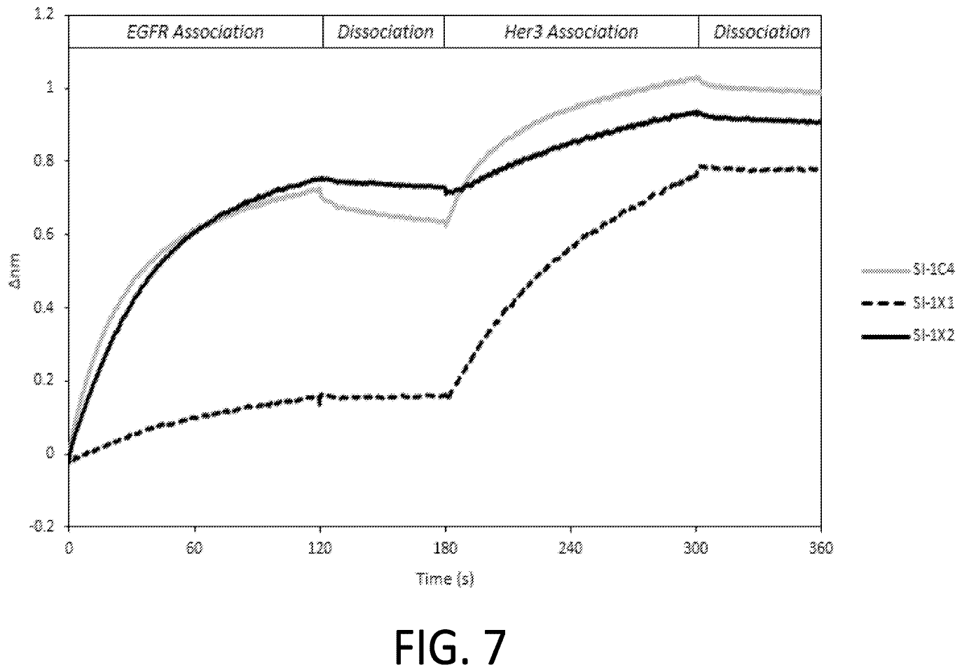

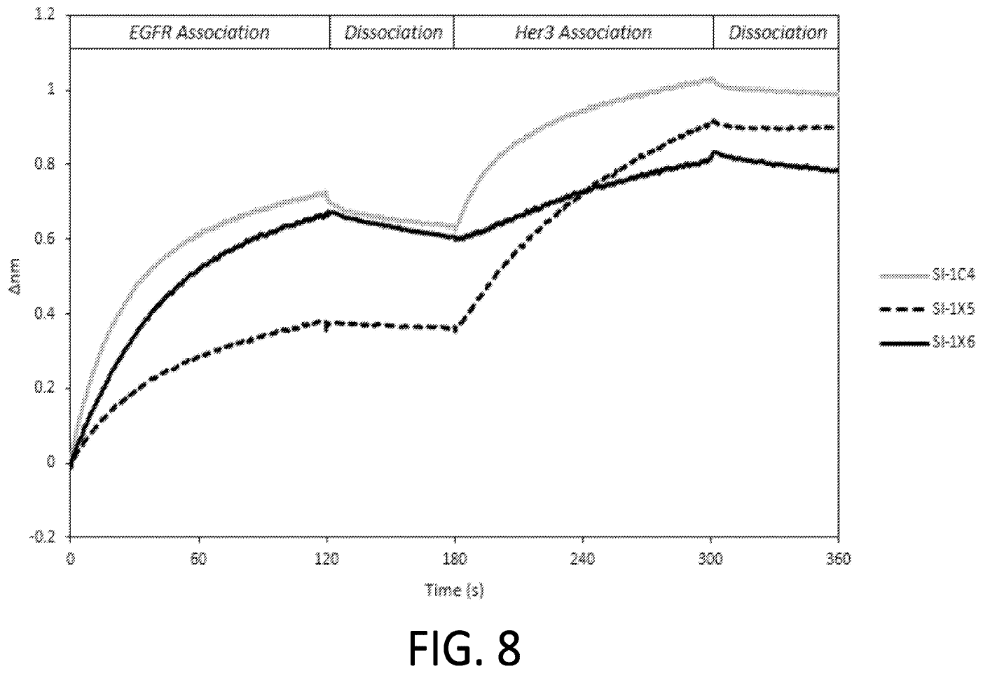

Bispecific binding to EGFR and Her3 extracellular domains was measured in a biolayer interferometry (BLI) binding assay on a BLItz instrument (ForteBio, Inc.). 200 nM of SI-1C1, SI-1C3, SI-1C4, SI-1C6, SI-1X1, SI-1X2, SI-1X3, SI-1X4, SI-1X5, and SI-1X6 were diluted in 1X Kinetics Buffer (ForteBio, Inc.) and captured on anti-huIgG Fc BLItz biosensor tips for 120 seconds. Tips were washed in KB for 30 seconds and moved to an EGFR sample (ProSpec Bio, PKA-344) for binding at 200 nM. Binding of EGFR ECD to the tips was recorded as biolayer interferometry signals (Anm) over an association time of 120 seconds. Tips were moved to KB and dissociation was observed for 60 seconds. The process was repeated with Her3 ECD sample (Sino Biological, 10201-H08H-10) at 200 nM for 120 seconds and a similar dissociation step of 60 seconds in KB. FIGS. 8-10 7-9 report data starting at the association step of EGFR to the antibody-loaded biosensor. Antibodies are able to exhibit simultaneous bispecific binding of EGFR and Her3 while being bound by the Fc to the sensor. As observed in FIG. 7 and FIG. 8, the display of the EGFR binding domain as Fab (SI-1X2, SI-1X6) has stronger on-rate binding than their scFv forms (SI-1X1, SI-1X5, respectively). Here, both EGFR and Her3 exhibit the same Fab>>scFv on-rate trend. SI-1X3 and SI-1X4 do not exhibit binding to monomeric EGFR, however each has the ability to bind Her3, as expected since each molecule uses the same Her3 binding domain as SI-1X1, SI-1X2, SI-1X5, and SI-1X6. SI-1X3 and SI-1X4 are investigated for dimeric EGFR binding in an ELISA below.

### Example 4: Dimeric EGFR ELISA Assay

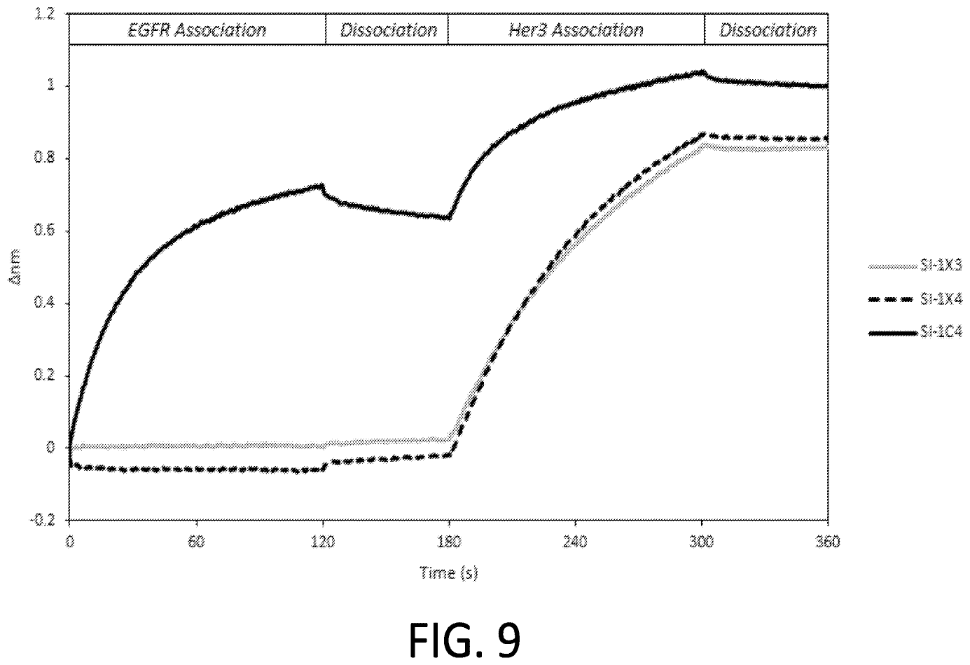

As observed earlier, SI-1X3 and SI-1X4 were unable to bind a monomeric form of EGFR in a BLI assay (FIG. 9). It has been suggested that in order for the αEGFR binding domain used in SI-1C5, SI-1X3, and SI-1X4 to bind to EGFR in vitro, bivalent binding is required (Perez et al, Chin Clin Oncol 2014; 3(1): 5). To observe this, we utilized ELISA for antibody binding relative to other EGFR binding antibodies using a dimeric form of EGFR.

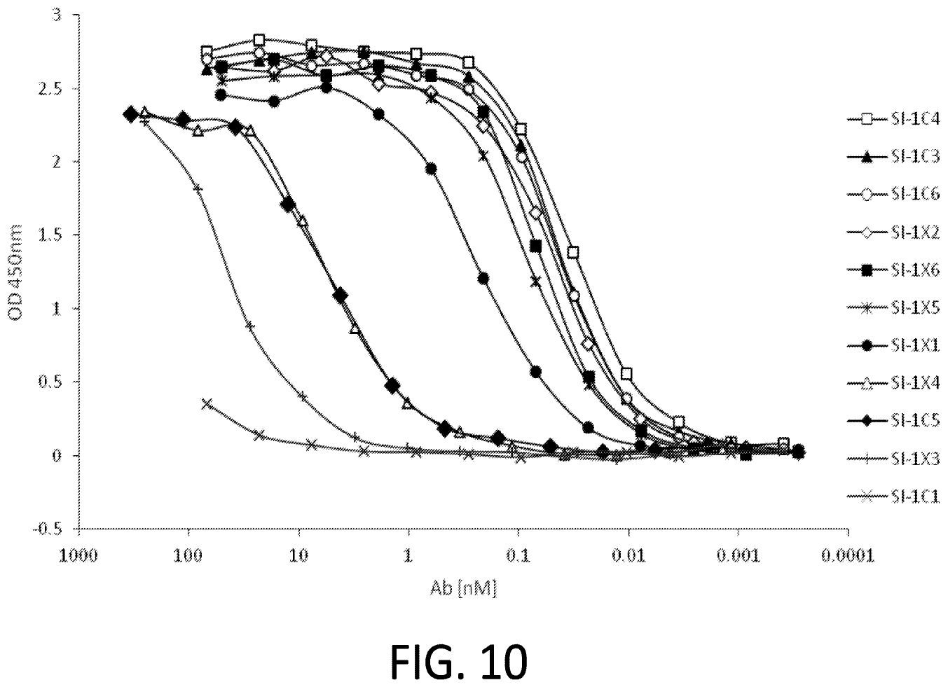

ELISA was performed using dimeric EGFR ECD reagent, SI-2C1, fused to rabbit Fc created in house. EGFR was coated onto Maxisorp immunoplates (Nunc) at 3 μg/mL in PBS at 4° C. overnight. Plates were blocked in PBS with 3% BSA and 0.05% Tween20 for 2 hours at room temperature. Antibodies were captured at starting at 10 μg/mL except for SI-1C5, SI-1X3, and SI-1X4 which started at 50 μg/mL for (reported in nM), all with 3× dilutions in PBST (1% BSA) for 1 hour at room temperature. Goat αhuman IgG-HRP antibody (Jackson ImmunoResearch, 109-035-098) was used for detection of the Fc portion of the antibodies at 1:2000 dilution in PBST (1% BSA) and developed in TMB (Thermo Scientific) for 5 minutes with 2M H2S04 as a stop solution. 3 washes with PBST (1% BSA) were performed between each step. All data points were performed in triplicate and collected at 450 nm (FIG. 10). SI-1C5, SI-1X3, and SI-1X4 all bound to the dimeric EGFR ECD in this ELISA format at high concentrations as compared to the other molecules.

### Example 5: Binding Kinetics of 1C5.2 and 1X4.2 Using Octet

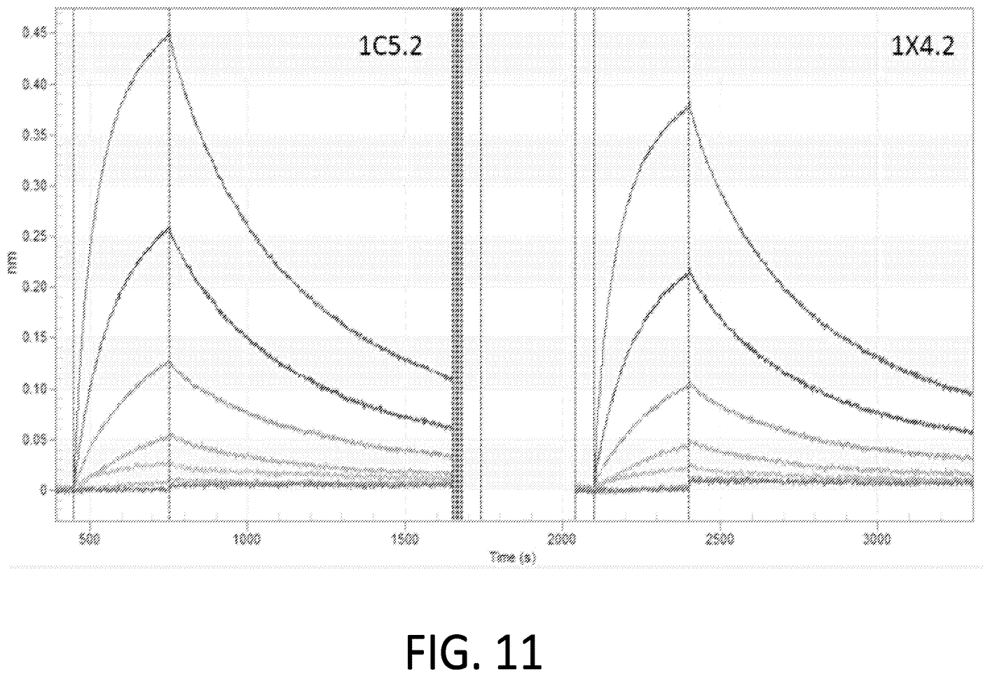

Kinetics determined using ForteBio Octet Red96 instrument with anti-human Fc sensors (ForteBio, AHC #18-5060). Binding experiments performed at 30° C. with 1000 RPM mixing. EGFR protein is extracellular domain (Met 1-Ser 645) of human EGFR with a C-terminal polyhistidine tag. All samples diluted in 10× Kinetics Buffer (ForteBio #18-5032). 1C5.2, 1X6 and 1X4.2 were loaded onto 8 sensors at 10 g/ml each for 300 seconds followed by a Baseline for 60 seconds in 10× Kinetics Buffer. Association with EGFR protein was performed for 300 seconds with each sensor in a single concentration of EGFR protein (300, 100, 33.33, 11.11, 3.705, 1.235, 0.4116 and 0 nM). Dissociation was then performed in 10× Kinetics Buffer for 900 seconds. A typical association and dissociation trace for 1C5.2 and 1X4.2 is shown in FIG. 11.

Data analysis was performed using ForteBio Data Analysis Software v9.0. Software curve-fitting was performed and the four most optimal curve fits for each 1C5.2 (TABLE 2), 1X4.2 (TABLE 3) and 1X6 (TABLE 4) were used and averaged to determine KD, k (on) and k (dis). The average KD for SI-1C5.2 and SI-1X4.2 were 19.2 nM and 18.4 nM respectively. The average KD for SI-1C6 was 3.04 nM 1C5.2 and 1X4.2 contained five amino acid changes as compared to 1C5 and 1X4 as described in example 1. These changes accounted for improved binding to EGFR ECD when compared to data generated for 1C5 and 1X4 in FIG. 10.

TABLE 2
Summary of KD, KON and KDIS for 1C5.2

| | EGFR | KD | KON | KDIS |
|---|---|---|---|---|
| | (NM) | (M) | (1/MS) | (1/S) |
| SI-1C5.2 | 300 | 3.74E−08 | 4.61E+04 | 1.72E−03 |
| SI-1C5.2 | 100 | 2.23E−08 | 7.89E+04 | 1.76E−03 |
| SI-1C5.2 | 33.3 | 9.94E−09 | 1.60E+05 | 1.59E−03 |
| SI-1C5.2 | 11.1 | 7.08E−09 | 2.12E+05 | 1.50E−03 |
| AVERAGES | | 1.92E−08 | 1.24E+05 | 1.64E−03 |

TABLE 3
Summary of KD, KON and KDIS for 1X4.2

| | EGFR | KD | KON | KDIS |
|---|---|---|---|---|
| | (NM) | (M) | (1/MS) | (1/S) |
| SI-1X4.2 | 300 | 3.69E−08 | 4.63E+04 | 1.71E−03 |
| SI-1X4.2 | 100 | 2.10E−08 | 7.88E+04 | 1.65E−03 |
| SI-1X4.2 | 33.3 | 9.44E−09 | 1.58E+05 | 1.49E−03 |
| SI-1X4.2 | 11.1 | 6.19E−09 | 2.18E+05 | 1.35E−03 |
| AVERAGES | | 1.84E−08 | 1.25E+05 | 1.55E−03 |

TABLE 4
Summary of KD, KON and KDIS for 1X6

| | EGFR | KD | KON | KDIS |
|---|---|---|---|---|
| | (NM) | (M) | (1/MS) | (1/S) |
| SI-1C6 | 300 | 3.04E−09 | 4.11E+05 | 1.25E−03 |
| SI-1C6 | 100 | 3.04E−09 | 4.11E+05 | 1.25E−03 |
| SI-1C6 | 33.3 | 3.04E−09 | 4.11E+05 | 1.25E−03 |
| SI-1C6 | 11.1 | 3.04E−09 | 4.11E+05 | 1.25E−03 |
| AVERAGES | | 3.04E−09 | 4.11E+05 | 1.25E−03 |

### Example 6: Binding Tests of Example Bispecific Antibodies to Tumor Cell Lines

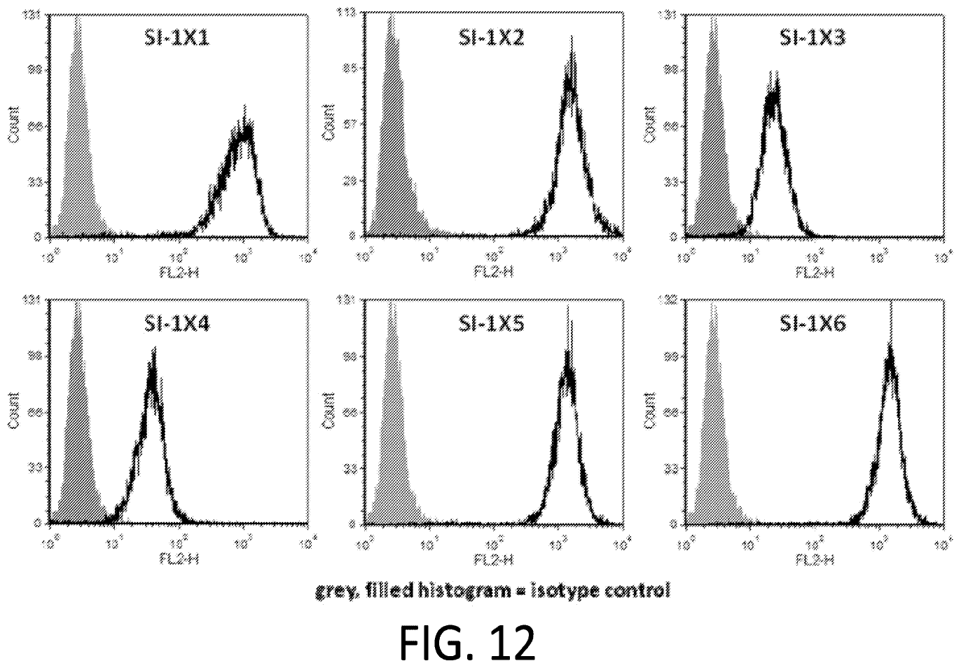

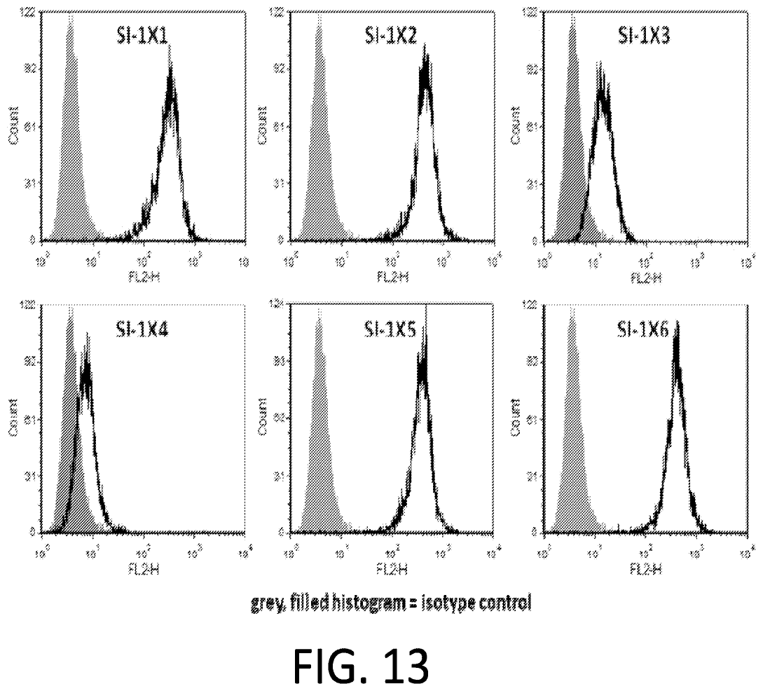

The bispecific antibodies SI-1X1, SI-1X2, SI-1X3, SI-1X4, SI-1X5, and SI-1X6, as well as an isotype control were tested for binding to the tumor cell lines, A431 (epidermoid carcinoma, ATCC CRL-1555) and BxPC3 (pancreatic adenocarcinoma, ATCC CRL-1687) by flow cytometry. Cells were grown in RPMI-1640 medium containing 10% fetal bovine serum and were harvested for analysis while in exponential growth phase. Aliquots of 5×10⁶ cells were washed once in PBS, then resuspended in 250 μl of PBS+1% bovine serum albumin (BSA) and incubated at 4° C. for 15 minutes to block membranes from non-specific binding. 250 μl of antibody, diluted to 10 μg/ml in PBS/1% BSA, was added to each sample for a final antibody concentration of 5 μg/ml. Cells were incubated in primary antibody for 1 hour at 4° C. with mixing. Cells were then washed twice with 1 ml PBS/1% BSA and then resuspended in 500 μl of PE-conjugated mouse-anti-human IgG-Fc and incubated at 4° C. with mixing for 45 minutes. Samples were again washed twice with 1 ml PBS/1% BSA, resuspended in 300 ml PBS and analyzed using a FACScalibur flow cytometer. For each sample, 10000 events were collected in the FL-2 channel. Histograms were generated using FCS Express software and SI-1X histograms were overlaid with histograms from the isotype control staining. All six bispecific antibodies displayed histogram shifts with respect to control staining indicating cell binding. This data is displayed in FIG. 12 (A431 cell binding) and FIG. 13 (BxPC3 cell binding).

### Example 7: Characterization of SI-1C5.2 and SI-1X4.2 by Cell Binding Assays

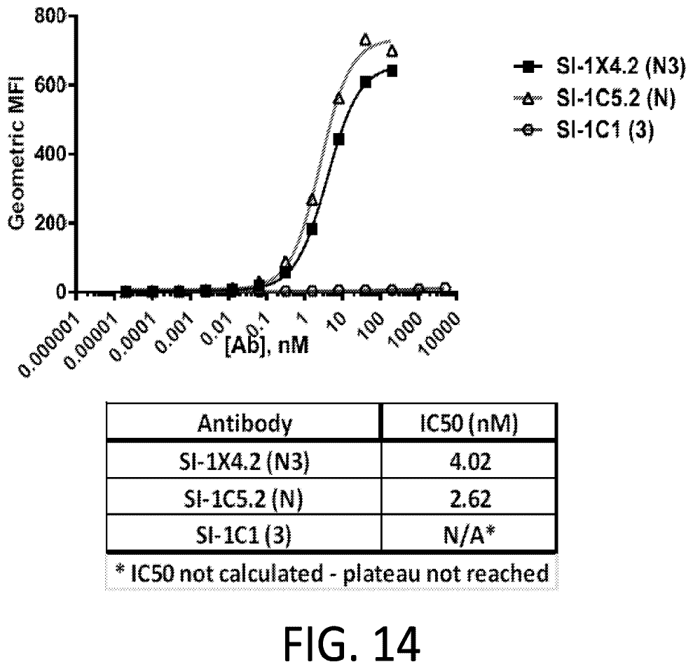

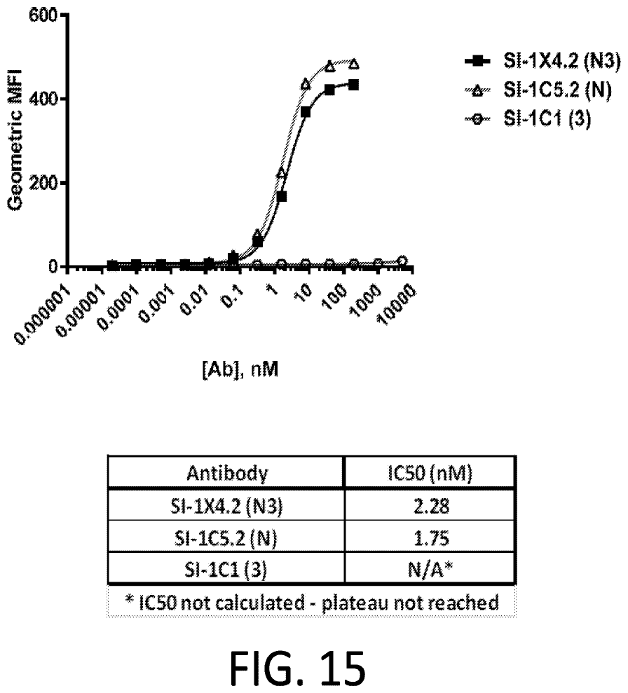

The bispecific antibody, SI-1X4.2, monospecific antibodies, SI-1C5.2 and SI-1C1, as well as an isotype control were tested for binding to the tumor cell lines, A431 (epidermoid carcinoma, ATCC CRL-1555) (FIG. 14) and FaDu (hypopharyngeal squamous cell carcinoma, ATCC HTB-43) (FIG. 15) by flow cytometry. Cells were grown in RPMI-1640 medium containing 10% fetal bovine serum and were harvested for analysis while in exponential growth phase. Cells were washed once in PBS, then resuspended in PBS+5% fetal bovine serum albumin (FBS) at a concentration of 5×10⁶ cells/ml and incubated at 4° C. for 15 minutes to block membranes from non-specific binding. 100 μl aliquots of cells were added to 100 μl aliquots of antibody (also diluted in PBS+5% FBS) in a 96-well plate. Samples were incubated in primary antibody for 45 minutes on ice. Cells were then washed twice with 200 μl of PBS+5% FBS and then resuspended in 100 μl of PE-conjugated mouse-anti-human IgG-Fc and incubated on ice 30 minutes. Samples were again washed twice with 200 μl of PBS+5% FBS, resuspended in 200 μl PBS and analyzed using a FACScalibur flow cytometer. For each sample, 10000 events were collected in the FL-2 channel. Histograms were analyzed using FCS Express software and the geometric mean fluorescence intensity (GMFI) was determined for each data set. EC50 binding values were determined by plotting the GMFI versus antibody concentration using Graphpad Prism software. The bispecific antibody, SI-1X4.2 displayed similar binding profile as the monospecific anti-EGFR antibody, SI-1C5.2 with similar EC50 in both cell lines. The other monospecific anti-Her3 antibody, SI-1C1 binds weakly to the two cell lines probably due to low level of expression of Her3 on the surface of the cells. 1C5.2 and 1X4.2 contained five amino acid changes as compared to 1C5 and 1X4 as described in example 1. These changes accounted for improved binding to target cells when compared to the parental molecule, 1X4.

### Example 8: Anti-Proliferative Effect of SI-1X Antibodies on Tumor Cell Lines

To assess the growth inhibitory potential of anti-Her3/EGFR bispecific antibodies, the effect on proliferation of A431 cells (ATCC CRL-1555, Manassas, Va.) which are an epidermoid carcinoma tumor line was tested. The effect on proliferation of BxPC3 (ATCC CRL-1687, Manassas, Va.), a pancreatic adenocarcinoma tumor line was also tested. For each line, cells were seeded into 96-well tissue culture plates at a density of 6000 cells/well in 100 μl RPMI-1640 medium containing 1% fetal bovine serum. After 4 hours, test antibodies were added at various concentrations, ranging from 0.0015 nM to 100 nM. Cells were cultured in the presence of test antibodies for 72 hours. To each well, 20 μl of MTS reagent (Promega, Madison, WI) was added and cells were incubated at 37° C. for 2 hours. MTS is readily taken up by actively proliferating cells, reduced into formazan (which readily absorbs light at 490 nm), and then secreted into the culture medium. Following incubation, OD490 values were measured using a BioTek (Winooski, VT) ELx800 absorbance reader. OD490 values for control cells (treated with medium only) were also obtained in this manner at the time of antibody addition in order to establish baseline metabolic activity. Proliferation may be calculated by subtracting the control baseline OD490 from the 72 hour OD490. Data from antibody titrations was expressed at % of control population according to the following formula: % of control proliferation=(test proliferation/control proliferation)*100.

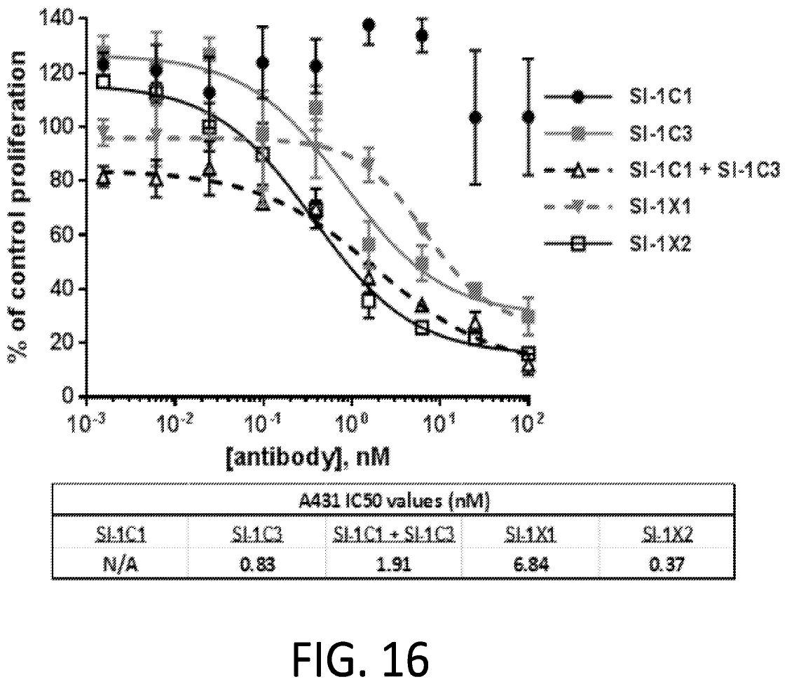

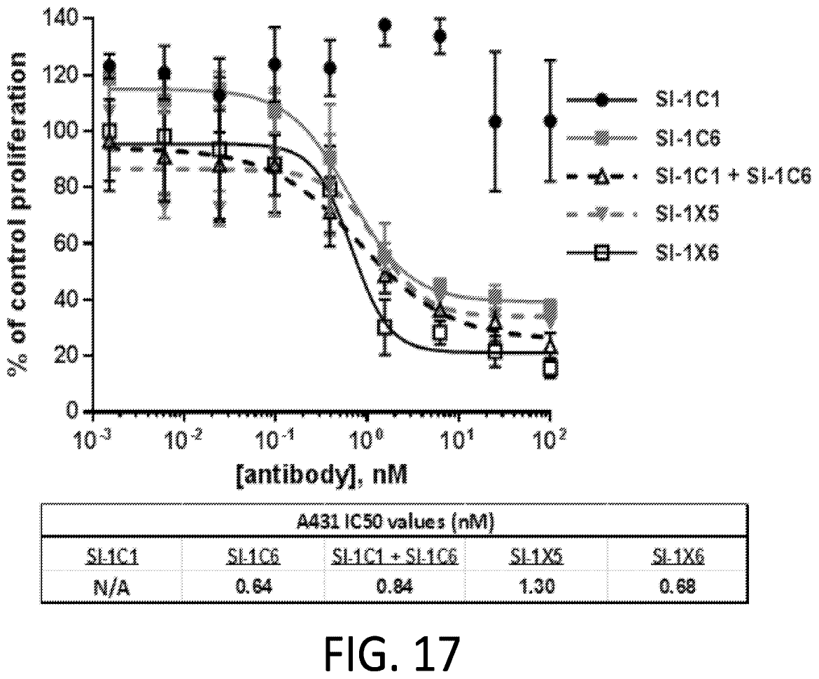

The effects of various bispecific anti-Her3/anti-EGFR antibodies on A431 cell proliferation are shown in FIG. 16 and FIG. 17. SI-1X2 demonstrated more efficacious antiproliferative effect than the control antibodies SI-1C1 (anti-Her3), SI-1C3 (anti-EGFR), or SI-1C1 and SI-1C3 applied together. SI-1X1 exhibited antiproliferative effects, although not to the degree seen with SI-1C3 and the combination of SI-1C1 and SI-1C3. Inhibition plots as well as IC50 values are shown in FIG. 17. Similar results were observed for SI-1X5 and SI-1X6, where SI-1X6 is more potent than SI-1X5 and the control antibody SI-1C1 (anti-Her3), however it displayed similar antiproliferative potential as the control antibody SI-1C6 (anti-EGFR) and the combination of SI-1C1 and SI-1C6. This may be seen along with IC50 values in FIG. 17.

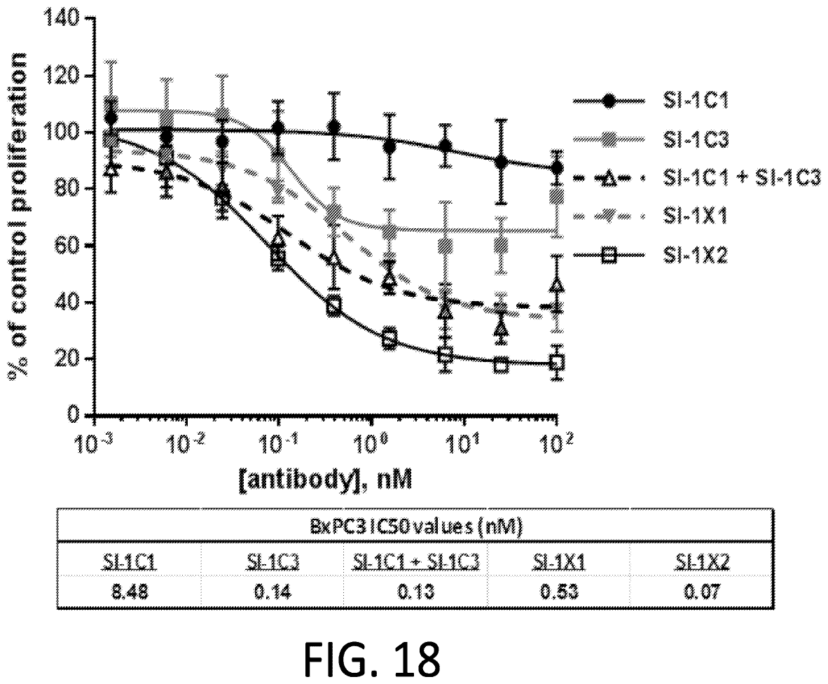

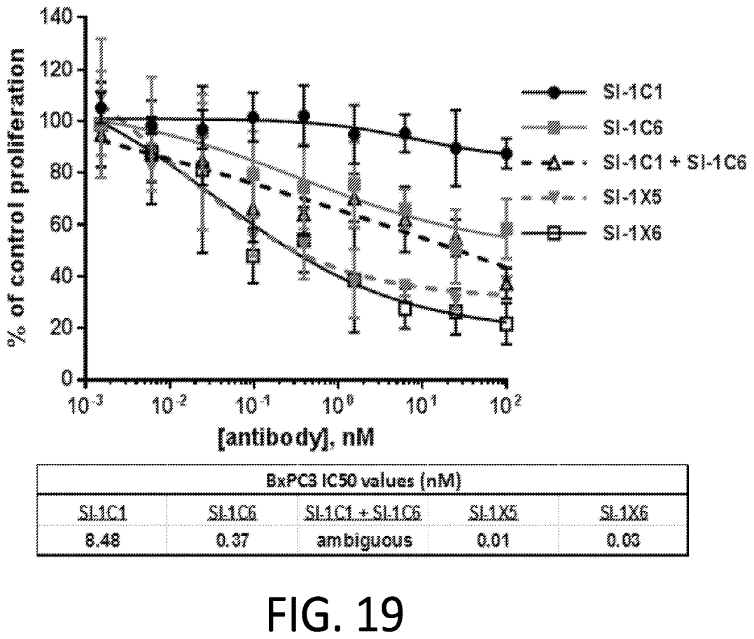

These molecules were also tested for antiproliferative effects in the BxPC3 cell line (FIG. 18 and FIG. 19). Again, SI-1X2 demonstrated more efficacious antiproliferative effect than the control antibodies SI-1C1 (anti-Her3), SI-1C3 (anti-EGFR), or SI-1C1 and SI-1C3 applied together. SI-1X1 was more efficacious than SI-1C1, but weaker than SI-1C3 and the combination of SI-1C1 and SI-1C3. Inhibition curves and IC50 values are displayed in FIG. 19. BxPC3 proliferation was more strongly inhibited by both SI-1X5 and SI-1X6 than with the control antibodies SI-1C1 (anti-Her3), SI-1C6 (anti-EGFR), or SI-1C1 and SI-1C6 in combination. This data along with IC50 values is shown in FIG. 19.

### Example 9: Anti-Proliferative Effect of SI-1C5.2 and SI-1X4.2 on Tumor Cell Lines

To assess the growth inhibitory potential of anti-Her3/EGFR bispecific antibodies, the effect on proliferation of FaDu (nasopharyngeal squamous cell carcinoma line, ATCC HTB-43) and A431 (epidermoid carcinoma, ATCC CRL-1555) cells were tested. Cells were seeded into 96-well tissue culture plates at a density of 6000 cells/well in 100 μl RPMI-1640 medium containing 1% fetal bovine serum. After 4 hours, test antibodies were added at various concentrations, ranging from 0.0015 nM to 100 nM. Cells were cultured in the presence of test antibodies for 72 hours. To each well, 11 μl of alamar blue reagent (Thermo Scientific) was added and cells were incubated at 37° C. for 2 hours. Alamar blue is readily taken up by actively proliferating cells, reduced, and then secreted into the culture medium. The reduced form of alamar blue is strongly fluorescent. Following incubation, fluorescence was measured using a Molecular Devices (Sunnyvale, CA) FilterMax F5 multi-mode plate reader using an excitation wavelength of 535 nm and an emission wavelength of 595 nm. Fluorescence values for control cells (treated with medium only) were also obtained in this manner at the time of antibody addition in order to establish baseline metabolic activity. Proliferation may be calculated by subtracting the control baseline fluorescence from the 72-hour fluorescence values. Data from antibody titrations was expressed at % of control population according to the following formula: % of control proliferation=(test proliferation/control proliferation)*100.

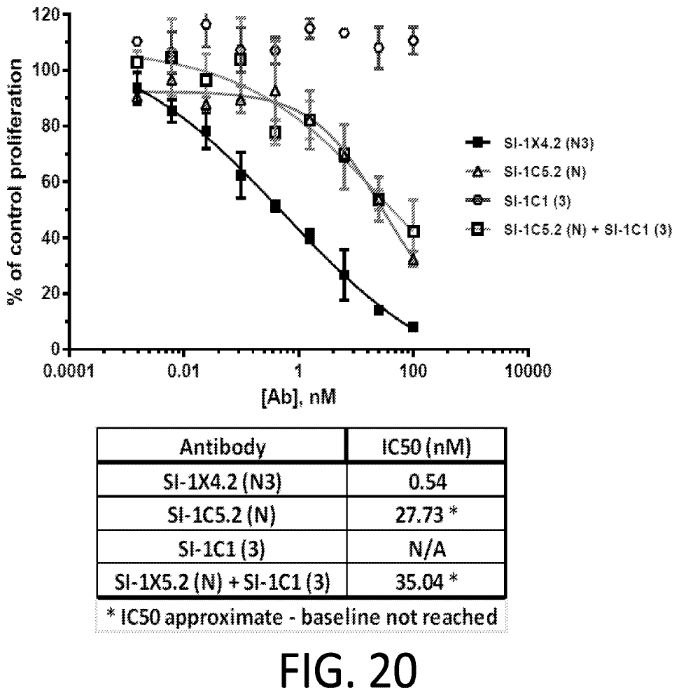

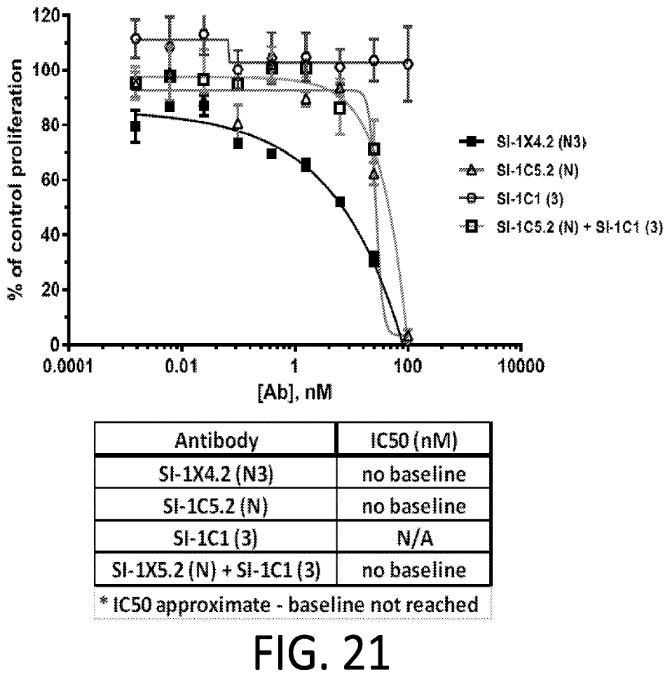

The effects of SI-1C5.2 and SI-1X4.2 on Fadu and A431 cell proliferation are shown in FIG. 20 and FIG. 21 respectively. In both cell lines, SI-1X4.2 demonstrated improved efficacious anti-proliferative effect than the control antibodies, SI-1C5.2 (anti-EGFR Mab), SI-1C1 (anti-Her3 Mab) or SI-1C1 and SI-1C7 applied together.

### Example 10: ADCC Activities of SI-1X Bispecific Antibodies

The ability of SI-1X antibodies to mediate cellular cytotoxicity against several tumor cell lines was tested. Whole blood was obtained from normal, healthy volunteers. Blood was diluted with an equal volume of phosphate buffered saline (PBS). 20 ml aliquots of diluted blood were carefully layered over 15 ml Ficol Pacque PLUS (GE Life Sciences cat #17-1440-02; Pittsburgh, PA). Tubes were centrifuged at 300 g for 40 minutes with no brake. Following centrifugation most of the plasma layer was carefully aspirated and the buffy coat (containing PBMC) was carefully removed with a pipet in the smallest possible volume. PBMCs were pooled in 50 ml tubes and PBS added to bring each tube up to 50 ml. Tubes were centrifuged at 1300 RPM for 10 minutes and the supernatant was carefully aspirated. Cells were resuspended in 40 ml PBS and centrifuged again. The process was repeated for a total of 2 washes. Following the final wash, cells were resuspended in 30 ml RPMI-1630+10% FBS and incubated overnight at 37° C., 5% CO₂.

Target cells tested were the head and neck squamous cell carcinoma line, Fa Du (ATCC HTB-43, Manassas, VA) and the non-small cell lung adenocarcinoma cell line, NCI-H1975 (ATCC CRL-5908, Manassas, VA). Target cells were labeled with calcein as follows. Cells were grown as monolayers and were detached by incubation with accutase. Cells were washed twice in RPMI with no serum. 1 ml of cells at 4×10⁶ cells/ml was mixed with 1 ml RPMI (no serum)+20 μM calcein AM (Sigma cat #C1359; St. Louis, MO). Cells were incubated at 37° C. for 30 minutes, with gentle mixing every 10 minutes. Following labeling, cells were washed twice with 14 ml RPMI+10% FBS+2.5 mM probenecid (assay medium). Probenecid (Sigma cat #P8761; St. Louis, MO) is an anionic transporter inhibitor and is known to reduce spontaneous release of intracellular calcein. Cells were resuspended in 20 ml assay medium and allowed to recover for 2 hours at 37° C., 5% CO₂. Cells were then washed once with assay medium and diluted to 200,000 cells/ml. Aliquots of 50 μl (10,000 cells) calcein-labeled cells were aliquoted to 96-well round-bottom plates. 50 μl of antibody (at 3× final concentration) was added to cells and allowed to bind for 40 minutes on ice. PBMCs from the previous day were centrifuged at 300 g for 5 minutes, resuspended in 20 ml fresh assay medium, counted, and diluted to 6×10⁶ cells/ml. 50 μl PBMC (300,000) were added to each well and plates incubated at 37° C., 5% CO₂ for 4 hours. Each antibody was titrated in triplicate via 10-fold serial dilutions, starting at 50 nM and going down to 0.00005 nM. Control wells were also set up containing labeled target cells in the absence of antibody and effector cells in order to measure maximal and spontaneous calcein release.

At the end of the 4-hour incubation, 50 μl of assay medium containing 8% IGEPAL CA-630 (Sigma cat #18896; St. Louis, MO) was added to control wells containing labeled target cells only (to measure the maximal calcein release). 50 μl of assay medium was added to all the other wells to bring the total volume to 200 μl per well. Plates were centrifuged at 2000 RPM for 10 minutes and 150 μl supernatant was carefully transferred to V-bottom 96-well plates. These plates were centrifuged at 2000 RPM for an additional 10 minutes and 100 ml supernatant was carefully transferred to black, clear-bottom 96-well plates. Calcein in the supernatant was quantitated by measuring the fluorescence of each sample using an excitation wavelength of 485 nM and an emission wavelength of 535 nM. The percentage of specific lysis was calculated as follows:

$$\% specific lysis = [ ( test sample value - spontaneous release ) / ( maximal release - spontaneous release ) ] * 100$$

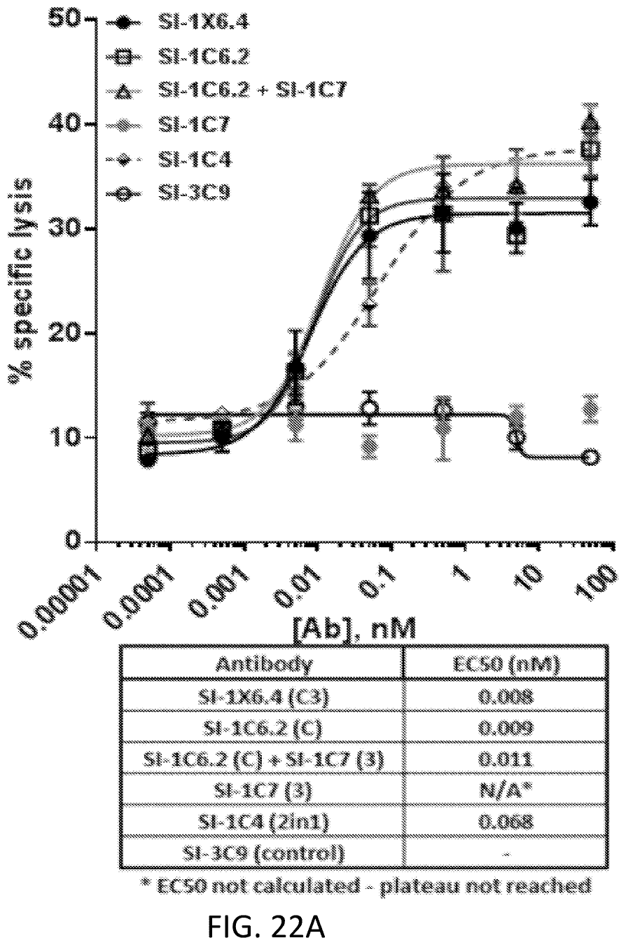

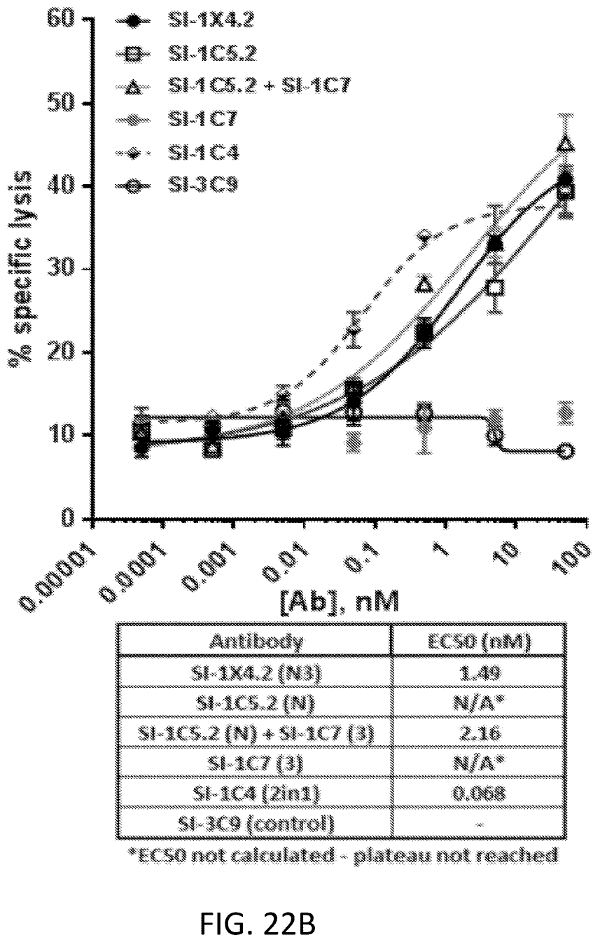

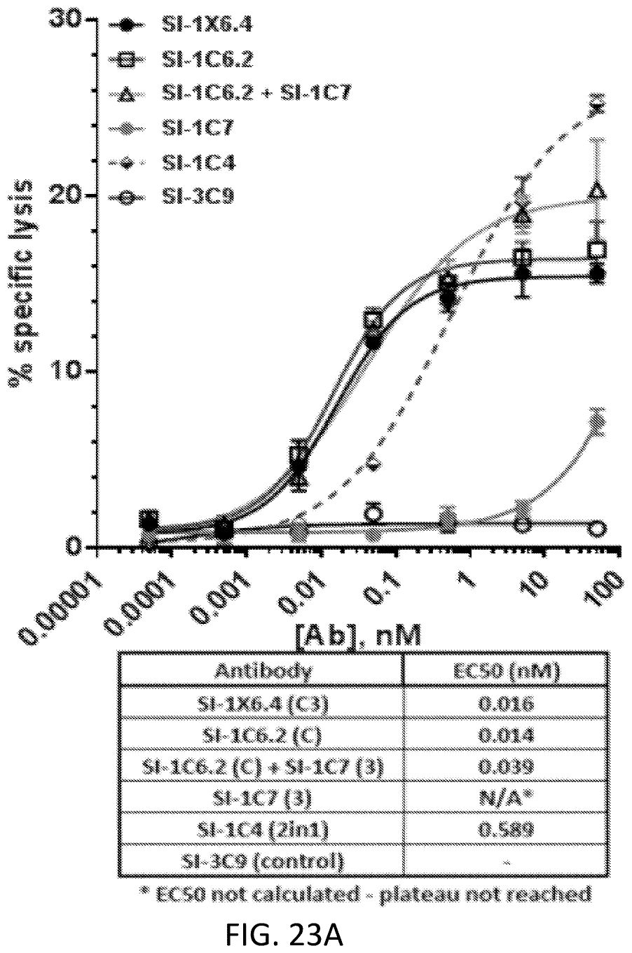

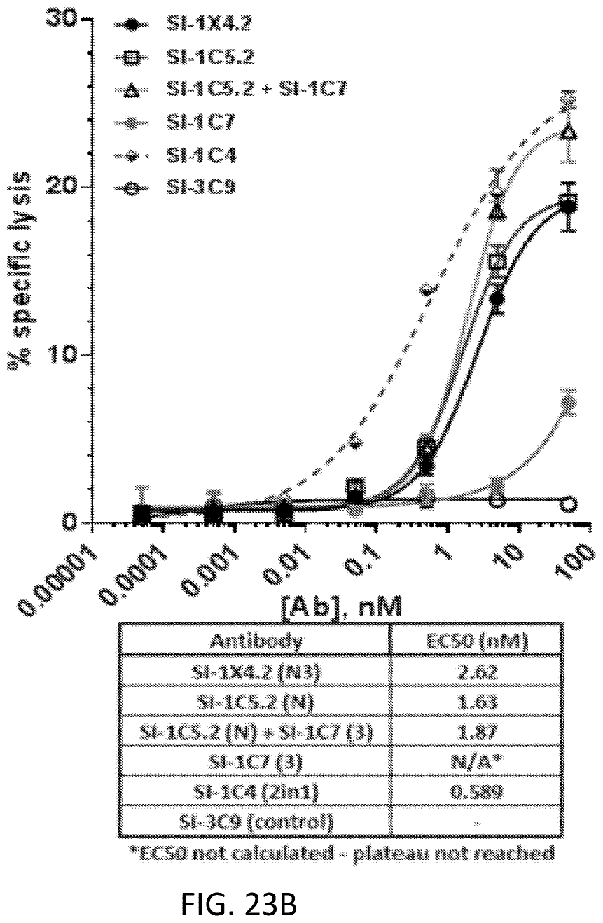

The data is shown in FIGS. 22A and 22B and FIGS. 23A and 23B. For both cell lines, SI-1X6.4 mediated cellular cytotoxicity, but was not particularly more effective than the control antibodies, SI-1C6.2, SI-1C7, or the combination of SI-1C6.2+SI-1C7. SI-1X6.4 did mediate cytotoxicity with a lower EC50 than our benchmark antibody, SI-1C4. For both cell lines, SI-1X4.2 mediated cellular cytotoxicity at about the same degree as the control antibodies. However, it was not as effective as mediating cellular cytotoxicity as the benchmark, SI-1C4. This is likely due to the lower affinity of SI-1X4.2.

### Example 11: Thermal Stability of SI-1X Bispecific Antibodies

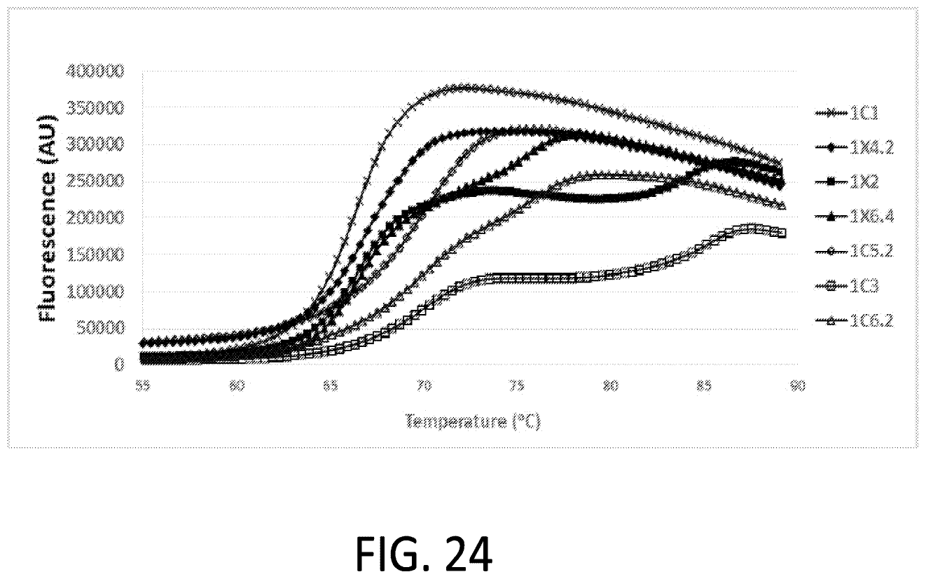

Protein Thermal Shift Study was performed for protein thermal stability analysis. Protein melt reactions were set up using Protein Thermal Shift Buffer™ and the Protein Thermal Shift Dye™ (Applied Biosystems). In brief, the 20 μl reaction mixture contains 5 μg protein, 5 μl Protein Thermal Shift Buffer™ and 2.5u 8× diluted Protein Thermal Shift™ Dye. For the negative control, PBS was used instead. The reaction mixture was added into MicroAmp Optical Reaction Plate and sealed with MicroAmp Optical Adhesive Film. Each sample consisted of 4 repeats. The protein melt reactions were run on Applied Biosystem Real-Time PCR System from 25-90° C. in 1% increment and then analyzed by Protein Thermal Shift Software™. FIG. 24 shows the thermal curve of SI-1X2, SI-1X4.2, SI-1X6.4, SI-1C3, SI-1C3, SI-1C6.2, SI-1C5.2 and SI-1C7. TABLE 5 shows Tm for these molecules. Tm is defined as the temperature needed to unfold 50% of the protein. The bispecific molecules, 1X2, 1X4.2 and 1X6 all have Tm around 66° C. which are comparable to all the MAbs (1C3, 1C6.2, 1C5.2) and the Fc-scFv (1C7) molecules.

TABLE 5

| Protein | Tm |
|---|---|
| Name | (° C.) |
| SI-1X2 | 66.52 |
| SI-1C3 | 70.06 |
| SI-1X4.2 | 66.94 |
| SI-1C5.2 | 70.26 |
| SI-1X6.4 | 66.50 |
| SI-1C6.2 | 70.12 |
| SI-1C7 | 66.40 |

### Example 12: Serum Stability of SI-1X Bispecific Antibodies

Serum stability of the molecules SI-1C5.2, SI-1C6.2, SI-1X4.2, and SI-1X6.4 was determined by comparative binding to monomeric EGFR ECD by ELISA after incubation at 100 μg/mL in 95% human serum (Atlanta Biologics, S40110) at 37° C. for Days 0, 3, and 7 time points with an extra time point of 55° C. on Day 7 to provide a known condition where degradation occurs. ELISA plates were coated with monomeric EGFR ECD (SI-2R4) at 3 μg/mL in PBS at 4° C. overnight. Coated ELISA plates were blocked with 3% BSA PBST for 2 hours at 25° C. and then washed 3 times with PBST. SI-1C6.2 and SI-1X6.4 were diluted 1:10 with 1% BSA PBST and diluted 4× across the plate. SI-1C5.2 and SI-1X4.2 were diluted 1:2 with 1% BSA PBST and diluted 4× across the plate and incubated at 25° C. for 1 hour. 3 more washes with PBST were performed before antigen capture with 1 μg/mL Her3 ECD Rabbit lgG1 (SI-1R1) for 1 hour at 25° C. in 1% BSA PBST. 3 more washes with PBST were performed before goat anti-rabbit IgG-HRP (Bio-Rad 172-1019) secondary antibody was applied at 1:5000 dilution in 1% BSA PBST at 25° C. for 1 hour. 3 final washes with PBST before development with 100 μl Pierce 1-step Ultra TMB ELISA (Pierce, 34028) for 10 minutes with a final quench of 100 μl 2M H₂SO₄. Plates were read at 450 nm. ELISA data was plotted and curves created using GraphPad Prism 6.

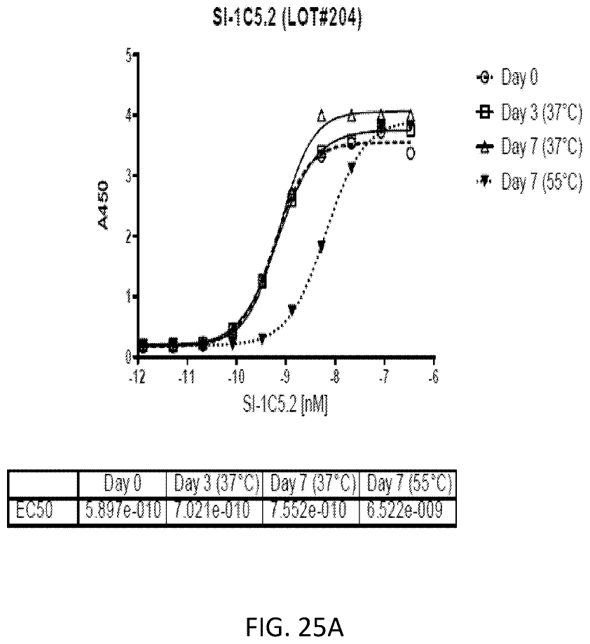

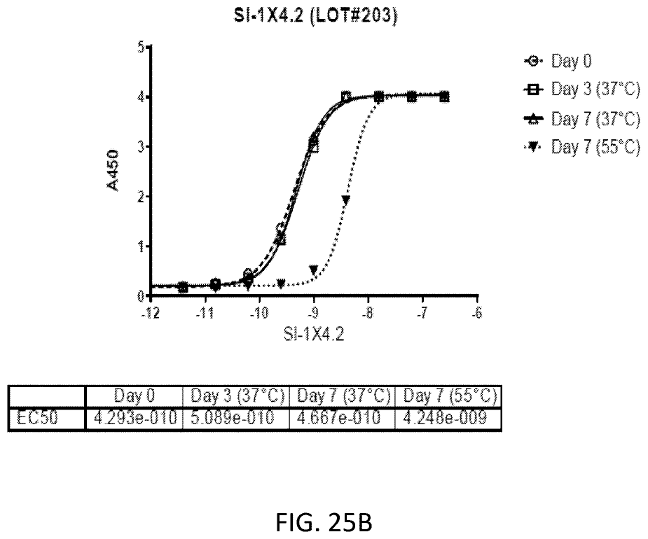

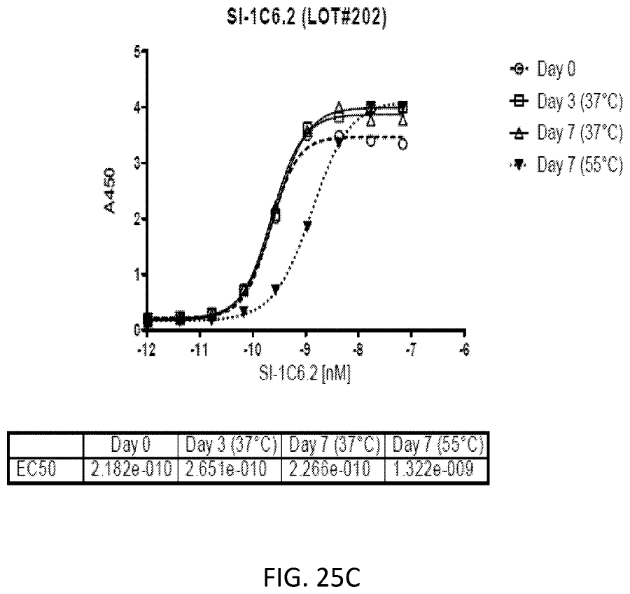

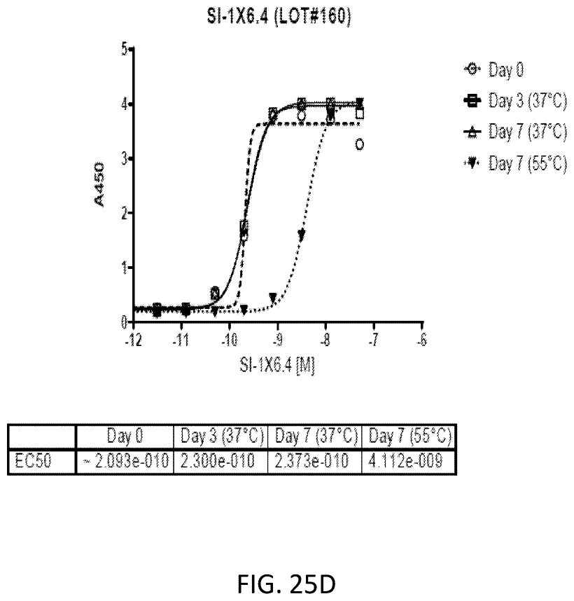

Results of the ELISA are reported by EC50 on FIGS. 25A-25D and indicate a favorable profile of minor degradation when held at 37° C. When placed in 55° C., the EC50 shifts roughly a log as the molecules are subjected to degradation conditions. EC50 values for SI-1C5.2 shift from 589.7 PM on Day 0 to 755.2 pM on Day 7 at 37° C. (Δ165.5 pM) with a shift to 6.522 nM on Day 7 at 55° C. (Δ5932.3 pM). EC50 values for SI-1C6 shift from 218.2 pM on Day 0 to 226.6 pM on Day 7 at 37° C. (Δ8.4 pM) with a shift to 1.322 nM on Day 7 at 55° C. (Δ1103 pM). EC50 values for SI-1X4.2 shift from 429.3 pM on Day 0 to 466.7 pM on Day 7 at 37° C. (Δ37.4 pM) with a shift to 4.248 nM on Day 7 at 55° C. (Δ3818.7 pM). EC50 values for SI-1X6 shift from 209.3 PM on Day 0 to 237.3 pM on Day 7 at 37° C. (Δ28 pM) with a shift to 4.112 nM on Day 7 at 55° C. (Δ3902.7 pM).

### Example 13: PK Half-Life of SI-1X Molecules

To test their half-life in vivo, pharmacokinetic experiments were performed in SD rats. A single, intravenous tail vein injection of bispecific Abs (1C6 10 mg/kg, 1X6 10 mg/kg, 1X2 10 mg/kg, 1X4 32 mg/kg) were given to groups of 4 female rats randomized by body weight (190-212 g range). Blood (˜150 μL) was drawn from the orbital plexus at each time point, processed for serum, and stored at −80° C. until analysis. Study durations were 28 days.

Antibody concentrations were determined using three ELISA assays. In assay 1 (EGFR ECD coated ELISA), recombinant EGFR-rabbit Fc was coated to the plate, wells were washed with PBST (phosphate buffered saline with 0.05% Tween) and blocked with 1% BSA in PBST. Serum or serum diluted standards were then added, followed by PBST washing, addition of HRP labeled rabbit-anti-human lgG (BOSTER), and additional PBST washing. TMB was then added and the plates were incubated 2.5 minutes in the dark. Color reaction was stopped by adding 2M sulfuric acid. Plate was read at 450 nm wavelength. For assay 2 (Her3 coated ELISA), serum was detected using a similar ELISA, but recombinant HER3-His was used as capture reagent. For assay 3 (Sandwich ELISA), recombinant HER3-His was coated, serum or serum diluted standard were added, followed by PBST washing, addition of EGFR-rabbit Fc in PBST, and additional PBST washing. HRP labeled goat-anti-rabbit lgG (BOSTER) was then added. PK parameters were determined with a non-compartmental model.

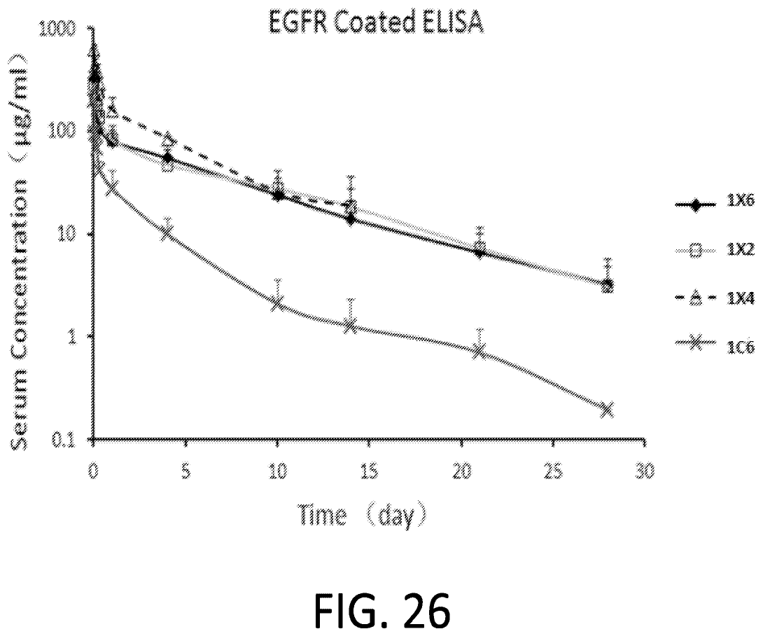

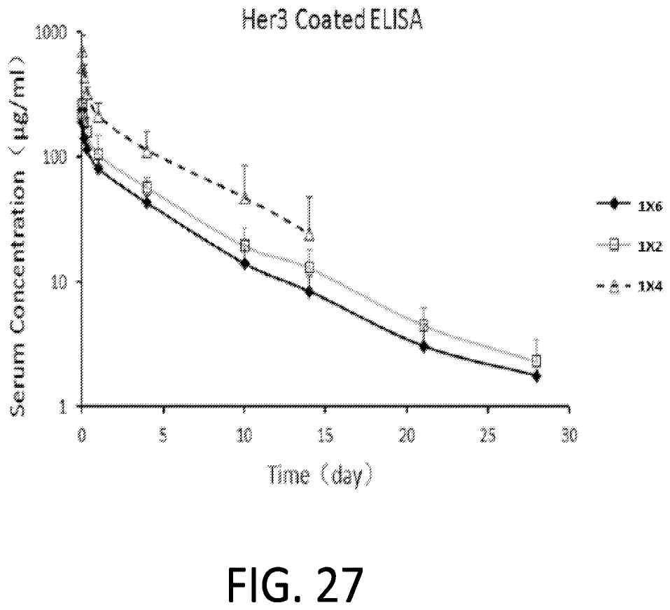

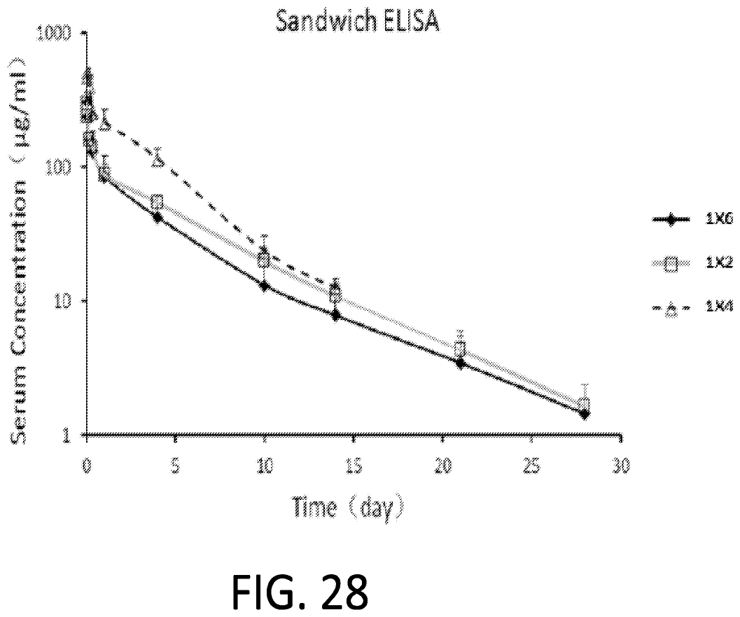

FIGS. 26-28 show serum concentration data for four antibodies with three different assays respectively. Fitted PK parameters from in vivo PK studies are provided in TABLE 6. PK data include half-life, which represents the beta phase that characterizes elimination of antibody from serum and Cmax, which represents the maximal observed serum concentration, AUC, which represents the area under the concentration time curve.

TABLE 6

| | | Half-Life | Cmax | AUC |
|---|---|---|---|---|
| Assay | Sample | (h) | (μg/ml) | (μg/ml*h) |
| EGFR | SI-1X6 | 159 | 326.5 | 18250.6 |
| Coated | SI-1X2 | 130 | 280.3 | 18889.8 |
| ELISA | SI-1X4.2 | 146 | 627.8 | 31317.0 |
| | SI-1C6 | 130 | 196.4 | 3790.3 |
| Her3 | SI-1X6 | 142 | 236.7 | 14213.6 |
| Coated | SI-1X2 | 136 | 264.8 | 19012.2 |
| ELISA | SI-1X4.2 | 124 | 716.6 | 40063.4 |
| Sandwich | SI-1X6 | 136 | 301.6 | 14182.6 |
| ELISA | SI-1X2 | 123 | 297.6 | 17203.9 |
| | SI-1X4.2 | 211 | 518.9 | 34874.6 |

### Example 14: Mouse Xenograft Studies

The example tested the activity of SI-1X2, SI-1X4.2 and SI-1X6 of concomitant blockade of EGFR, HER3 in preclinical models of Fadu (head and neck squamous cell carcinoma xenograft model) and compared their potency with cetuximab and cetuximab in combination with an anti-HER3 antibody.

All mouse studies were conducted through Institutional Animal care and used committee-approved animal protocols in accordance with institutional guidelines. Six-week-old female Balb/c Nude mice were purchased from Beijing Vital River Laboratories and housed in air-filtered laminar flow cabinets with a 12-hour light cycle and food and water ad libitum. The size of the animal groups was calculated to measure means difference between placebo and treatment groups of 25% with a power of 80% and a P value of 0.01. Host mice carrying xenografts were randomly and equally assigned to either control or treatment groups. Animal experiments were conducted in a controlled and non-blinded manner. For cell line-derived xenograft studies, mice were injected subcutaneously with 2×106 Fadu suspended 150 μl of culture medium per mouse.

Once tumors reached an average volume of 100-250 mm3, mice were randomized into 9 groups, with 6 mice per group. Vehicle Control, 1C6 (25 mg/kg), 1C4 (25 mg/kg), 1C6+1C1 (25 mg/kg+50 mg/kg), SI-1X2 (25 mg/kg), SI-1X6 (10 mg/kg), SI-1X6 (25 mg/kg), and SI-1X4.2 (10 mg/kg) SI-1X4 (25 mg/kg). All test articles were administered once weekly via intravenous injection. Tumors were measured by digital caliper over the entire treatment period every 3 days and the volume was determined using the following formula: ½×lenth×width2. The body weight of mice were recorded before the first dose and followed by every week during the treatment period and recovery period.

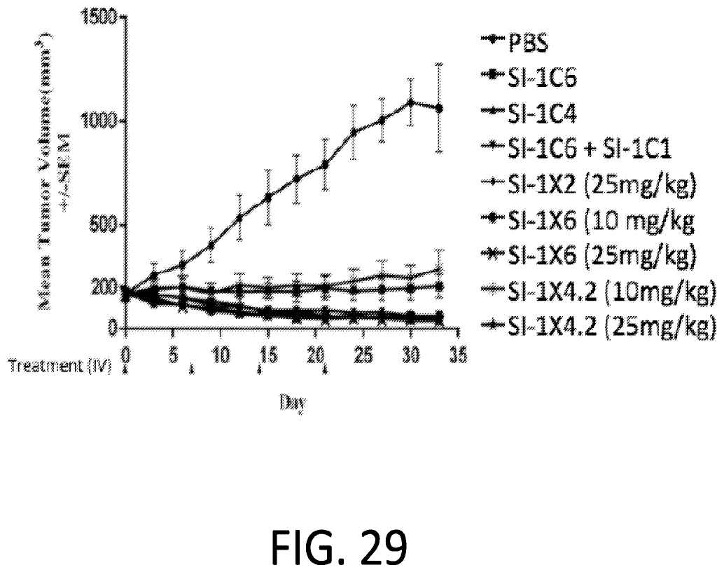

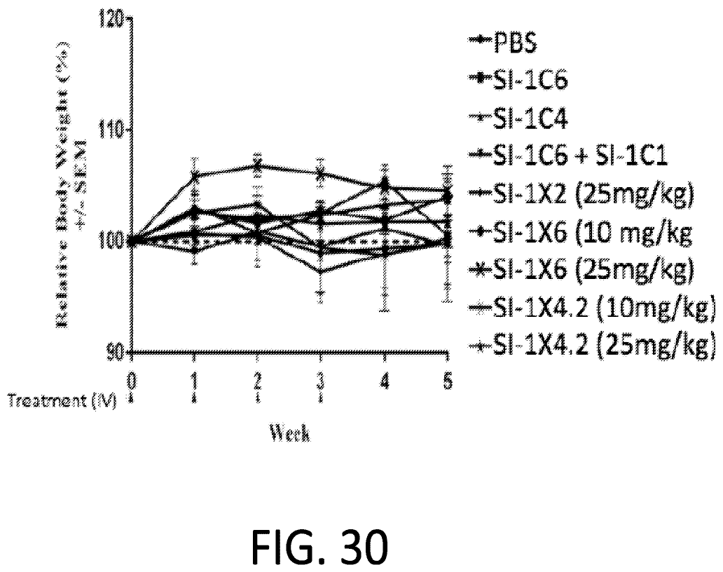

All the test groups of SI-1X2, SI-1X6 and SI-1X4.2 and SI-1X6 combination yielded significantly tumor growth inhibition compared to positive control of SI-1C6 excluding the low dose SI-1X4.2 10 mg/kg group (FIGS. 29-30). Moreover, no relapses were observed 2 weeks after treatment cessation excluding the low dose SI-1X4.2 10 mg/kg group.

### Pharmaceutical Compositions

The term “effective amount” refers to an amount of a drug effective to achieve a desired effect, e.g., to ameliorate disease in a subject. Where the disease is a caner, the effective amount of the drug may inhibit (for example, slow to some extent, inhibit or stop) one or more of the following example characteristics including, without limitation, cancer cell growth, cancer cell proliferation, cancer cell motility, cancer cell infiltration into peripheral organs, tumor metastasis, and tumor growth. Wherein the disease is a caner, the effective amount of the drug may alternatively do one or more of the following when administered to a subject: slow or stop tumor growth, reduce tumor size (for example, volume or mass), relieve to some extent one or more of the symptoms associated with the cancer, extend progression free survival, result in an objective response (including, for example, a partial response or a complete response), and increase overall survival time. To the extent the drug may prevent growth and/or kill existing cancer cells, it is cytostatic and/or cytotoxic.

With respect to the formulation of suitable compositions for administration to a subject such as a human patient in need of treatment, the antibodies disclosed herein may be mixed or combined with pharmaceutically acceptable carriers known in the art dependent upon the chosen route of administration. There are no particular limitations to the modes of application of the antibodies disclosed herein, and the choice of suitable administration routes and suitable compositions are known in the art without undue experimentation.

Although many forms of administration are possible, an example administration form would be a solution for injection, in particular for intravenous or intra-arterial injection. Usually, a suitable pharmaceutical composition for injection may include pharmaceutically suitable carriers or excipients such as, without limitation, a buffer, a surfactant, or a stabilizer agent. Example buffers may include, without limitation, acetate, phosphate or citrate buffer. Example surfactants may include, without limitation, polysorbate. Example stabilizer may include, without limitation, human albumin.

Similarly, persons skilled in the art have the ability to determine the effective amount or concentration of the antibodies disclosed therein to effective treat a condition such as a cancer. Other parameters such as the proportions of the various components in the pharmaceutical composition, administration does and frequency may be obtained by person skilled in the art without undue experimentation. For example, a suitable solution for injection may contain, without limitation, from about 1 to about 20, from about 1 to about 10 mg antibodies per ml. The example dose may be, without limitation, from about 0.1 to about 20, from about 1 to about 5 mg/Kg body weight. The example administration frequency could be, without limitation, once per day or three times per week.

While the present disclosure has been described with reference to particular embodiments or examples, it may be understood that the embodiments are illustrative and that the disclosure scope is not so limited. Alternative embodiments of the present disclosure may become apparent to those having ordinary skill in the art to which the present disclosure pertains. Such alternate embodiments are considered to be encompassed within the scope of the present disclosure. Accordingly, the scope of the present disclosure is defined by the appended claims and is supported by the foregoing description.

Sequence listing:

```
SI-1C1 SEQUENCES
SI1C1 LIGHT CHAIN FULL-LENGTH NUCLEOTIDE SEQUENCE (SEQ ID NO: 139)
CAGTCTGCCCTGACTCAGCCTGCCTCCGTGTCTGGGTCTCCTGGACAGTCGATCACCATCTCCTGCAC
TGGAACCAGCAGTGACGTTGGTGGTTATAACTTTGTCTCCTGGTACCAACAACACCCAGGCAAAGCC
CCCAAACTCATGATCTATGATGTCAGTGATCGGCCCTCAGGGGTGTCTGATCGCTTCTCCGGCTCCAA
GTCTGGCAACACGGCCTCCCTGATCATCTCTGGCCTCCAGGCTGACGACGAGGCTGATTATTACTGC
AGCTCATATGGGAGCAGCAGCACTCATGTGATTTTCGGCGGAGGGACCAAGCTGACCGTCCTACGT
ACGGTGGCTGCACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGT
SI-1C1 LIGHT CHAIN VARIABLE LIGHT CHAIN NUCLEOTIDE SEQUENCE (SEQ ID NO: 140)
CAGTCTGCCCTGACTCAGCCTGCCTCCGTGTCTGGGTCTCCTGGACAGTCGATCACCATCTCCTGCAC
TGGAACCAGCAGTGACGTTGGTGGTTATAACTTTGTCTCCTGGTACCAACAACACCCAGGCAAAGCC
CCCAAACTCATGATCTATGATGTCAGTGATCGGCCCTCAGGGGTGTCTGATCGCTTCTCCGGCTCCAA
GTCTGGCAACACGGCCTCCCTGATCATCTCTGGCCTCCAGGCTGACGACGAGGCTGATTATTACTGC
AGCTCATATGGGAGCAGCAGCACTCATGTGATTTTCGGCGGAGGGACCAAGCTGACCGTCCTA
SI-1C1 LIGHT CHAIN FULL-LENGTH AMINO ACID SEQUENCE (SEQ ID NO: 141). HUMAN
KAPPA CONSTANT DOMAIN IS UNDERLINED (SEQ ID NO: 207)
QSALTQPASVSGSPGQSITISCTGTSSDVGGYNFVSWYQQHPGKAPKLMIYDVSDRPSGVSDRFSGSKSG
NTASLIISGLQADDEADYYCSSYGSSSTHVIFGGGTKLTVLRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKSE
NRGEC
SI-1C1 LIGHT CHAIN VARIABLE LIGHT CHAIN AMINO ACID SEQUENCE (SEQ ID NO: 142).
COMPLEMENTARITY DETERMINING REGIONS ARE UNDERLINED (SEQ ID NOS: 208, 209 AND 210)
QSALTQPASVSGSPGQSITISCTGTSSDVGGYNFVSWYQQHPGKAPKLMIYDVSDRPSGVSDRFSGSKSG
NTASLIISGLQADDEADYYCSSYGSSSTHVIFGGGTKLTVL
SI1C1 HEAVY CHAIN FULL-LENGTH NUCLEOTIDE SEQUENCE (SEQ ID NO: 143)
CAGGTGCAGCTGCAGGAGTCGGGGGGAGGCCTGGTCAAGCCTGGAGGGTCCCTGAGACTCTCCTGT
GCAGCCTCTGGATTCACCTTTAGTAGTTATTGGATGAGCTGGGTCCGCCAGGCTCCAGGGAAGGGG
CTGGAGTGGGTGGCCAACATAAACCGCGATGGAAGTGCGAGTTACTATGTGGACTCTGTGAAGGGC
CGATTCACCATCTCCAGAGACGACGCCAAGAACTCACTGTATCTGCAAATGAACAGCCTGAGAGCTG
AGGACACGGCTGTGTATTACTGTGCGAGAGATCGTGGGGTGGGCTACTTCGATCTCTGGGGCCGTG
GCACCCTGGTCACCGTCTCGAGCGCTAGCACCAAGGGCCCATCGGTCTTCCCCCTGGCACCCTCCTCC
AAGAGCACCTCTGGGGGCACAGCGGCCCTGGGCTGCCTGGTCAAGGACTACTTCCCCGAACCGGTG
ACGGTGTCGTGGAACTCAGGCGCCCTGACCAGCGGCGTGCACACCTTCCCGGCTGTCCTACAGTCCT
CAGGACTCTACTCCCTCAGCAGCGTGGTGACCGTGCCCTCCAGCAGCTTGGGCACCCAGACCTACAT
CTGCAACGTGAATCACAAGCCCAGCAACACCAAGGTGGACAAGAGAGTTGAGCCCAAATCTTGTGA
CAAAACTCACACATGCCCACCGTGCCCAGCACCTGAACTCCTGGGGGGACCGTCAGTCTTCCTCTTCC
CCCCAAAACCCAAGGACACCCTCATGATCTCCCGGACCCCTGAGGTCACATGCGTGGTGGTGGACGT
GAGCCACGAAGACCCTGAGGTCAAGTTCAACTGGTACGTGGACGGCGTGGAGGTGCATAATGCCAA
GACAAAGCCGCGGGAGGAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTACACCCTGCCCCCATCCC
GGGAGGAGATGACCAAGAACCAGGTCAGCCTGACCTGCCTGGTCAAAGGCTTCTATCCCAGCGACA
TCGCCGTGGAGTGGGAGAGCAATGGGCAGCCGGAGAACAACTACAAGACCACGCCTCCCGTGCTG
GACTCCGACGGCTCCTTCTTCCTCTATAGCAAGCTCACCGTGGACAAGAGCAGGTGGCAGCAGGGG
AACGTCTTCTCATGCTCCGTGATGCATGAGGCTCTGCACAACCACTACACGCAGAAGAGCCTCTCCCT
GTCTCCGGGTAAA
SI-1C1 HEAVY CHAIN VARIABLE HEAVY CHAIN NUCLEOTIDE SEQUENCE (SEQ ID NO: 144)
CAGGTGCAGCTGCAGGAGTCGGGGGGAGGCCTGGTCAAGCCTGGAGGGTCCCTGAGACTCTCCTGT
GCAGCCTCTGGATTCACCTTTAGTAGTTATTGGATGAGCTGGGTCCGCCAGGCTCCAGGGAAGGGG
CTGGAGTGGGTGGCCAACATAAACCGCGATGGAAGTGCGAGTTACTATGTGGACTCTGTGAAGGGC
CGATTCACCATCTCCAGAGACGACGCCAAGAACTCACTGTATCTGCAAATGAACAGCCTGAGAGCTG
AGGACACGGCTGTGTATTACTGTGCGAGAGATCGTGGGGTGGGCTACTTCGATCTCTGGGGCCGTG
GCACCCTGGTCACCGTCTCGAGC
SI-1C1 HEAVY CHAIN FULL-LENGTH AMINO ACID SEQUENCE (SEQ ID NO: 145). HUMAN
GAMMA-1 DOMAIN IS UNDERLINED (SEQ ID NO: 211)
QVQLQESGGGLVKPGGSLRLSCAASGFTFSSYWMSWVRQAPGKGLEWVANINRDGSASYYVDSVKGR
FTISRDDAKNSLYLQMNSLRAEDTAVYYCARDRGVGYFDLWGRGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPS
NTKVDKRVEPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGK
SI-1C1 HEAVY CHAIN VARIABLE HEAVY CHAIN AMINO ACID SEQUENCE (SEQ ID NO: 146).
COMPLEMENTARITY DETERMINING REGIONS ARE UNDERLINED (SEQ ID NOS: 212, 213 AND 214)
QVQLQESGGGLVKPGGSLRLSCAASGFTFSSYWMSWVRQAPGKGLEWVANINRDGSASYYVDSVKGR
FTISRDDAKNSLYLQMNSLRAEDTAVYYCARDRGVGYFDLWGRGTLVTVSS
SI-1X1 SEQUENCES
SI1X1 LIGHT CHAIN FULL-LENGTH NUCLEOTIDE SEQUENCE (SEQ ID NO: 147)
CAGTCTGCCCTGACTCAGCCTGCCTCCGTGTCTGGGTCTCCTGGACAGTCGATCACCATCTCCTGCAC
TGGAACCAGCAGTGACGTTGGTGGTTATAACTTTGTCTCCTGGTACCAACAACACCCAGGCAAAGCC
CCCAAACTCATGATCTATGATGTCAGTGATCGGCCCTCAGGGGTGTCTGATCGCTTCTCCGGCTCCAA
GTCTGGCAACACGGCCTCCCTGATCATCTCTGGCCTCCAGGCTGACGACGAGGCTGATTATTACTGC
AGCTCATATGGGAGCAGCAGCACTCATGTGATTTTCGGCGGAGGGACCAAGCTGACCGTCCTACGT
ACGGTGGCTGCACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGT
SI-1X1 LIGHT CHAIN VARIABLE LIGHT CHAIN NUCLEOTIDE SEQUENCE (SEQ ID NO: 148)
CAGTCTGCCCTGACTCAGCCTGCCTCCGTGTCTGGGTCTCCTGGACAGTCGATCACCATCTCCTGCAC
TGGAACCAGCAGTGACGTTGGTGGTTATAACTTTGTCTCCTGGTACCAACAACACCCAGGCAAAGCC
CCCAAACTCATGATCTATGATGTCAGTGATCGGCCCTCAGGGGTGTCTGATCGCTTCTCCGGCTCCAA
GTCTGGCAACACGGCCTCCCTGATCATCTCTGGCCTCCAGGCTGACGACGAGGCTGATTATTACTGC
AGCTCATATGGGAGCAGCAGCACTCATGTGATTTTCGGCGGAGGGACCAAGCTGACCGTCCTA
SI-1X1 LIGHT CHAIN FULL-LENGTH AMINO ACID SEQUENCE (SEQ ID NO: 149). HUMAN
KAPPA CONSTANT DOMAIN IS UNDERLINED (SEQ ID NO: 215)
QSALTQPASVSGSPGQSITISCTGTSSDVGGYNFVSWYQQHPGKAPKLMIYDVSDRPSGVSDRFSGSKSG
NTASLIISGLQADDEADYYCSSYGSSSTHVIFGGGTKLTVLRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKSF
NRGEC
SI-1X1 LIGHT CHAIN VARIABLE LIGHT CHAIN AMINO ACID SEQUENCE (SEQ ID NO: 150).
COMPLEMENTARITY DETERMINING REGIONS ARE UNDERLINED (SEQ ID NOS: 216, 217 AND
218)
QSALTQPASVSGSPGQSITISCTGTSSDVGGYNFVSWYQQHPGKAPKLMIYDVSDRPSGVSDRFSGSKSG
NTASLIISGLQADDEADYYCSSYGSSSTHVIFGGGTKLTVL
SI1X1 BISPECIFIC HEAVY CHAIN FULL-LENGTH NUCLEOTIDE SEQUENCE (SEQ ID NO: 151)
CAGGTGCAGCTGCAGGAGTCGGGGGGAGGCCTGGTCAAGCCTGGAGGGTCCCTGAGACTCTCCTGT
GCAGCCTCTGGATTCACCTTTAGTAGTTATTGGATGAGCTGGGTCCGCCAGGCTCCAGGGAAGGGG
CTGGAGTGGGTGGCCAACATAAACCGCGATGGAAGTGCGAGTTACTATGTGGACTCTGTGAAGGGC
CGATTCACCATCTCCAGAGACGACGCCAAGAACTCACTGTATCTGCAAATGAACAGCCTGAGAGCTG
AGGACACGGCTGTGTATTACTGTGCGAGAGATCGTGGGGTGGGCTACTTCGATCTCTGGGGCCGTG
GCACCCTGGTCACCGTCTCGAGCGCTAGCACCAAGGGCCCATCGGTCTTCCCCCTGGCACCCTCCTCC
AAGAGCACCTCTGGGGGCACAGCGGCCCTGGGCTGCCTGGTCAAGGACTACTTCCCCGAACCGGTG
ACGGTGTCGTGGAACTCAGGCGCCCTGACCAGCGGCGTGCACACCTTCCCGGCTGTCCTACAGTCCT
CAGGACTCTACTCCCTCAGCAGCGTGGTGACCGTGCCCTCCAGCAGCTTGGGCACCCAGACCTACAT
CTGCAACGTGAATCACAAGCCCAGCAACACCAAGGTGGACAAGAGAGTTGAGCCCAAATCTTGTGA
CAAAACTCACACATGCCCACCGTGCCCAGCACCTGAACTCCTGGGGGGACCGTCAGTCTTCCTCTTCC
CCCCAAAACCCAAGGACACCCTCATGATCTCCCGGACCCCTGAGGTCACATGCGTGGTGGTGGACGT
GAGCCACGAAGACCCTGAGGTCAAGTTCAACTGGTACGTGGACGGCGTGGAGGTGCATAATGCCAA
GACAAAGCCGCGGGAGGAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTACACCCTGCCCCCATCCC
GGGAGGAGATGACCAAGAACCAGGTCAGCCTGACCTGCCTGGTCAAAGGCTTCTATCCCAGCGACA
TCGCCGTGGAGTGGGAGAGCAATGGGCAGCCGGAGAACAACTACAAGACCACGCCTCCCGTGCTG
GACTCCGACGGCTCCTTCTTCCTCTATAGCAAGCTCACCGTGGACAAGAGCAGGTGGCAGCAGGGG
AACGTCTTCTCATGCTCCGTGATGCATGAGGCTCTGCACAACCACTACACGCAGAAGAGCCTCTCCCT
GTCTCCGGGTAAAGGCGGTGGAGGATCCGGCGGTGGTGGATCACAGGTGCAGCTGCAGGAGTCGG
GCCCAGGACTGGTGAAGCCTTCGGAGACCCTGTCCCTCACCTGCACTGTCTCTGGTGGCTCCGTCAG
CAGTGGTGATTACTACTGGACCTGGATACGGCAGTCCCCAGGGAAGGGACTGGAGTGGATTGGACA
CATCTATTACAGTGGGAACACCAATTATAACCCCTCCCTCAAGAGCCGACTCACCATATCAATTGACA
CGTCCAAGACTCAGTTCTCCCTGAAGCTGAGTTCTGTGACCGCTGCGGACACGGCCATTTATTACTGT
GTGCGAGATCGAGTGACTGGTGCTTTTGATATCTGGGGCCAAGGGACAATGGTCACCGTCTCGAGC
GGTGGAGGCGGTTCAGGCGGAGGTGGTTCCGGCGGTGGCGGCTCCGACATCCAGATGACCCAGTC
TCCATCCTCCCTGTCTGCATCTGTAGGAGACAGAGTCACCATCACTTGCCAGGCGAGTCAGGACATC
AGCAACTATTTAAATTGGTATCAGCAGAAACCAGGGAAAGCCCCTAAACTCCTGATCTACGATGCAT
CCAATTTGGAAACAGGGGTCCCATCAAGGTTCAGTGGAAGTGGATCTGGGACAGATTTTACTTTCAC
CATCAGCAGCCTGCAGCCTGAAGATATTGCAACATATTTCTGTCAACACTTTGATCATCTCCCGCTCG
CTTTCGGCGGAGGGACCAAGGTGGAAATTAAACGT
SI-1X1 BISPECIFIC HEAVY CHAIN VARIABLE HEAVY CHAIN NUCLEOTIDE SEQUENCE (SEQ ID NO: 152)
CAGGTGCAGCTGCAGGAGTCGGGGGGAGGCCTGGTCAAGCCTGGAGGGTCCCTGAGACTCTCCTGT
GCAGCCTCTGGATTCACCTTTAGTAGTTATTGGATGAGCTGGGTCCGCCAGGCTCCAGGGAAGGGG
CTGGAGTGGGTGGCCAACATAAACCGCGATGGAAGTGCGAGTTACTATGTGGACTCTGTGAAGGGC
CGATTCACCATCTCCAGAGACGACGCCAAGAACTCACTGTATCTGCAAATGAACAGCCTGAGAGCTG
AGGACACGGCTGTGTATTACTGTGCGAGAGATCGTGGGGTGGGCTACTTCGATCTCTGGGGCCGTG
GCACCCTGGTCACCGTCTCGAGC
SI-1X1 BISPECIFIC HEAVY CHAIN SCFV NUCLEOTIDE SEQUENCE (SEQ ID NO: 153)
CAGGTGCAGCTGCAGGAGTCGGGCCCAGGACTGGTGAAGCCTTCGGAGACCCTGTCCCTCACCTGC
ACTGTCTCTGGTGGCTCCGTCAGCAGTGGTGATTACTACTGGACCTGGATACGGCAGTCCCCAGGGA
AGGGACTGGAGTGGATTGGACACATCTATTACAGTGGGAACACCAATTATAACCCCTCCCTCAAGAG
CCGACTCACCATATCAATTGACACGTCCAAGACTCAGTTCTCCCTGAAGCTGAGTTCTGTGACCGCTG
CGGACACGGCCATTTATTACTGTGTGCGAGATCGAGTGACTGGTGCTTTTGATATCTGGGGCCAAGG
GACAATGGTCACCGTCTCGAGCGGTGGAGGCGGTTCAGGCGGAGGTGGTTCCGGCGGTGGCGGCT
CCGACATCCAGATGACCCAGTCTCCATCCTCCCTGTCTGCATCTGTAGGAGACAGAGTCACCATCACT
TGCCAGGCGAGTCAGGACATCAGCAACTATTTAAATTGGTATCAGCAGAAACCAGGGAAAGCCCCT
AAACTCCTGATCTACGATGCATCCAATTTGGAAACAGGGGTCCCATCAAGGTTCAGTGGAAGTGGAT
CTGGGACAGATTITACTTTCACCATCAGCAGCCTGCAGCCTGAAGATATTGCAACATATTTCTGTCAA
CACTTTGATCATCTCCCGCTCGCTTTCGGCGGAGGGACCAAGGTGGAAATTAAACGT
SI-1X1 BISPECIFIC HEAVY CHAIN FULL-LENGTH AMINO ACID SEQUENCE (SEQ ID NO: 154).
HUMAN GAMMA-1 DOMAIN IS UNDERLINED (SEQ ID NO: 219), CONNECTOR IS IN ITALICS
(SEQ ID NO: 220), SCFV IS IN BOLD (SEQ ID NO: 221)
QVQLQESGGGLVKPGGSLRLSCAASGFTFSSYWMSWVRQAPGKGLEWVANINRDGSASYYVDSVKGR
FTISRDDAKNSLYLQMNSLRAEDTAVYYCARDRGVGYFDLWGRGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPS
NTKVDKRVEPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGKGGGGSGGGGSQVQLQESGPGLVKPSETLSLTCTVSGGS
VSSGDYYWTWIRQSPGKGLEWIGHIYYSGNTNYNPSLKSRLTISIDTSKTQFSLKLSSVTAADTAIYYCV
RDRVTGAFDIWGQGTMVTVSSGGGGSGGGGSGGGGSDIQMTQSPSSLSASVGDRVTITCQASQDIS
NYLNWYQQKPGKAPKLLIYDASNLETGVPSRFSGSGSGTDFTFTISSLQPEDIATYFCQHFDHLPLAFGG
GTKVEIKR
SI-1X1 BISPECIFIC HEAVY CHAIN VARIABLE HEAVY CHAIN AMINO ACID SEQUENCE (SEQ ID
NO: 155). COMPLEMENTARITY DETERMINING REGIONS ARE UNDERLINED (SEQ ID NOS: 222,
223 AND 224)
QVQLQESGGGLVKPGGSLRLSCAASGFTFSSYWMSWVRQAPGKGLEWVANINRDGSASYYVDSVKGR
FTISRDDAKNSLYLQMNSLRAEDTAVYYCARDRGVGYFDLWGRGTLVTVSS
SI1X1 BISPECIFIC HEAVY CHAIN SCFV AMINO ACID SEQUENCE (SEQ ID NO: 156). ORDER: VH
(SEQ ID NO: 225)-LINKER (SEQ ID NO: 226)-VL (SEQ ID NO: 227). COMPLEMENTARITY
DETERMINING REGIONS ARE UNDERLINED (SEQ ID NOS: 228, 229, 230, 231, 232 AND 233).
LINKER IS IN BOLD ITALICS (SEQ ID NO: 226)
QVQLQESGPGLVKPSETLSLTCTVSGGSVSSGDYYWTWIRQSPGKGLEWIGHIYYSGNTNYNPSLKSRLTI
SIDTSKTQFSLKLSSVTAADTAIYYCVRDRVTGAFDIWGQGTMVTVSSGGGGSGGGGSGGGGSDIQMT
QSPSSLSASVGDRVTITCQASQDISNYLNWYQQKPGKAPKLLIYDASNLETGVPSRFSGSGSGTDFTFTISS
LQPEDIATYFCQHFDHLPLAFGGGTKVEIKR
SI-1X2 SEQUENCES
SI1X2 LIGHT CHAIN FULL-LENGTH NUCLEOTIDE SEQUENCE (SEQ ID NO: 157)
GACATCCAGATGACCCAGTCTCCATCCTCCCTGTCTGCATCTGTAGGAGACAGAGTCACCATCACTTG
CCAGGCGAGTCAGGACATCAGCAACTATTTAAATTGGTATCAGCAGAAACCAGGGAAAGCCCCTAA
ACTCCTGATCTACGATGCATCCAATTTGGAAACAGGGGTCCCATCAAGGTTCAGTGGAAGTGGATCT
GGGACAGATTTTACTTTCACCATCAGCAGCCTGCAGCCTGAAGATATTGCAACATATTTCTGTCAACA
CTTTGATCATCTCCCGCTCGCTTTCGGCGGAGGGACCAAGGTGGAAATTAAACGTACGGTGGCTGCA
CCATCTGTCTTCATCTTCCCGCCATCTGATGAGCAGTTGAAATCTGGAACTGCCTCTGTTGTGTGCCTG
CTGAATAACTTCTATCCCAGAGAGGCCAAAGTACAGTGGAAGGTGGATAACGCCCTCCAATCGGGTA
ACTCCCAGGAGAGTGTCACAGAGCAGGACAGCAAGGACAGCACCTACAGCCTCAGCAGCACCCTGA
CGCTGAGCAAAGCAGACTACGAGAAACACAAAGTCTACGCCTGCGAAGTCACCCATCAGGGCCTGA
GCTCGCCCGTCACAAAGAGCTTCAACAGGGGAGAGTGT
SI-1X2 LIGHT CHAIN VARIABLE LIGHT CHAIN NUCLEOTIDE SEQUENCE (SEQ ID NO: 158)
GACATCCAGATGACCCAGTCTCCATCCTCCCTGTCTGCATCTGTAGGAGACAGAGTCACCATCACTTG
CCAGGCGAGTCAGGACATCAGCAACTATTTAAATTGGTATCAGCAGAAACCAGGGAAAGCCCCTAA
ACTCCTGATCTACGATGCATCCAATTTGGAAACAGGGGTCCCATCAAGGTTCAGTGGAAGTGGATCT
GGGACAGATTTTACTTTCACCATCAGCAGCCTGCAGCCTGAAGATATTGCAACATATTTCTGTCAACA
CTTTGATCATCTCCCGCTCGCTTTCGGCGGAGGGACCAAGGTGGAAATTAAA
SI-1X2 LIGHT CHAIN FULL-LENGTH AMINO ACID SEQUENCE (SEQ ID NO: 159). HUMAN
KAPPA CONSTANT DOMAIN IS UNDERLINED (SEQ ID NO: 234)
DIQMTQSPSSLSASVGDRVTITCQASQDISNYLNWYQQKPGKAPKLLIYDASNLETGVPSRFSGSGSGTD
FTFTISSLQPEDIATYFCQHFDHLPLAFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVYACEVTHQGLSSPVTKSFNRGE
C
SI-1X2 LIGHT CHAIN VARIABLE LIGHT CHAIN AMINO ACID SEQUENCE (SEEQ ID NO: 160).
COMPLEMENTARITY DETERMINING REGIONS ARE UNDERLINED (SEQ ID NOS: 236, 236 AND
237)
DIQMTQSPSSLSASVGDRVTITCQASQDISNYLNWYQQKPGKAPKLLIYDASNLETGVPSRFSGSGSGTD
FTFTISSLQPEDIATYFCQHFDHLPLAFGGGTKVEIK
SI1X2 BISPECIFIC HEAVY CHAIN FULL-LENGTH NUCLEOTIDE SEQUENCE (SEQ ID NO: 161)
CAGGTGCAGCTGCAGGAGTCGGGCCCAGGACTGGTGAAGCCTTCGGAGACCCTGTCCCTCACCTGC
ACTGTCTCTGGTGGCTCCGTCAGCAGTGGTGATTACTACTGGACCTGGATCCGGCAGTCCCCAGGGA
AGGGACTGGAGTGGATTGGACACATCTATTACAGTGGGAACACCAATTATAACCCCTCCCTCAAGAG
CCGACTCACCATATCAATTGACACGTCCAAGACTCAGTTCTCCCTGAAGCTGAGTTCTGTGACCGCTG
CGGACACGGCCATTTATTACTGTGTGCGAGATCGAGTGACTGGTGCTTTTGATATCTGGGGCCAAGG
GACAATGGTCACCGTCTCGAGCGCTAGCACCAAGGGCCCATCGGTCTTCCCCCTGGCACCCTCCTCCA
AGAGCACCTCTGGGGGCACAGCGGCCCTGGGCTGCCTGGTCAAGGACTACTTCCCCGAACCGGTGA
CGGTGTCGTGGAACTCAGGCGCCCTGACCAGCGGCGTGCACACCTTCCCGGCTGTCCTACAGTCCTC
AGGACTCTACTCCCTCAGCAGCGTGGTGACCGTGCCCTCCAGCAGCTTGGGCACCCAGACCTACATC
TGCAACGTGAATCACAAGCCCAGCAACACCAAGGTGGACAAGAGAGTTGAGCCCAAATCTTGTGAC
AAAACTCACACATGCCCACCGTGCCCAGCACCTGAACTCCTGGGGGGACCGTCAGTCTTCCTCTTCCC
CCCAAAACCCAAGGACACCCTCATGATCTCCCGGACCCCTGAGGTCACATGCGTGGTGGTGGACGTG
AGCCACGAAGACCCTGAGGTCAAGTTCAACTGGTACGTGGACGGCGTGGAGGTGCATAATGCCAAG
ACAAAGCCGCGGGAGGAGCAGTACAACAGCACGTACCGTGTGGTCAGCGTCCTCACCGTCCTGCAC
CAGGACTGGCTGAATGGCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTACACCCTGCCCCCATCCC
GGGAGGAGATGACCAAGAACCAGGTCAGCCTGACCTGCCTGGTCAAAGGCTTCTATCCCAGCGACA
TCGCCGTGGAGTGGGAGAGCAATGGGCAGCCGGAGAACAACTACAAGACCACGCCTCCCGTGCTG
GACTCCGACGGCTCCTTCTTCCTCTATAGCAAGCTCACCGTGGACAAGAGCAGGTGGCAGCAGGGG
AACGTCTTCTCATGCTCCGTGATGCATGAGGCTCTGCACAACCACTACACGCAGAAGAGCCTCTCCCT
GTCTCCGGGTAAAGGCGGTGGAGGATCCGGCGGTGGTGGATCACAGGTGCAGCTGCAGGAGTCGG
GGGGAGGCCTGGTCAAGCCTGGAGGGTCCCTGAGACTCTCCTGTGCAGCCTCTGGATTCACCTTTAG
TAGTTATTGGATGAGCTGGGTCCGCCAGGCTCCAGGGAAGGGGCTGGAGTGGGTGGCCAACATAA
ACCGCGATGGAAGTGCGAGTTACTATGTGGACTCTGTGAAGGGCCGATTCACCATCTCCAGAGACG
ACGCCAAGAACTCACTGTATCTGCAAATGAACAGCCTGAGAGCTGAGGACACGGCTGTGTATTACTG
TGCGAGAGATCGTGGGGTGGGCTACTTCGATCTCTGGGGCCGTGGCACCCTGGTCACCGTCTCGAG
CGGTGGAGGCGGTTCAGGCGGAGGTGGTTCCGGCGGTGGCGGCTCCCAGTCTGCCCTGACTCAGCC
TGCCTCCGTGTCTGGGTCTCCTGGACAGTCGATCACCATCTCCTGCACTGGAACCAGCAGTGACGTTG
GTGGTTATAACTTTGTCTCCTGGTACCAACAACACCCAGGCAAAGCCCCCAAACTCATGATCTATGAT
GTCAGTGATCGGCCCTCAGGGGTGTCTGATCGCTTCTCCGGCTCCAAGTCTGGCAACACGGCCTCCC
TGATCATCTCTGGCCTCCAGGCTGACGACGAGGCTGATTATTACTGCAGCTCATATGGGAGCAGCAG
CACTCATGTGATTTTCGGCGGAGGGACCAAGGTGACCGTCCTA
SI-1X2 BISPECIFIC HEAVY CHAIN VARIABLE HEAVY CHAIN NUCLEOTIDE SEQUENCE (SEQ ID
NO: 162)
CAGGTGCAGCTGCAGGAGTCGGGCCCAGGACTGGTGAAGCCTTCGGAGACCCTGTCCCTCACCTGC
ACTGTCTCTGGTGGCTCCGTCAGCAGTGGTGATTACTACTGGACCTGGATCCGGCAGTCCCCAGGGA
AGGGACTGGAGTGGATTGGACACATCTATTACAGTGGGAACACCAATTATAACCCCTCCCTCAAGAG
CCGACTCACCATATCAATTGACACGTCCAAGACTCAGTTCTCCCTGAAGCTGAGTTCTGTGACCGCTG
CGGACACGGCCATTTATTACTGTGTGCGAGATCGAGTGACTGGTGCTTTTGATATCTGGGGCCAAGG
GACAATGGTCACCGTCTCGAGC
SI-1X2 BISPECIFIC HEAVY CHAIN SCFV NUCLEOTIDE SEQUENCE (SEQ ID NO: 163)
CAGGTGCAGCTGCAGGAGTCGGGGGGAGGCCTGGTCAAGCCTGGAGGGTCCCTGAGACTCTCCTGT
GCAGCCTCTGGATTCACCTTTAGTAGTTATTGGATGAGCTGGGTCCGCCAGGCTCCAGGGAAGGGG
CTGGAGTGGGTGGCCAACATAAACCGCGATGGAAGTGCGAGTTACTATGTGGACTCTGTGAAGGGC
CGATTCACCATCTCCAGAGACGACGCCAAGAACTCACTGTATCTGCAAATGAACAGCCTGAGAGCTG
AGGACACGGCTGTGTATTACTGTGCGAGAGATCGTGGGGTGGGCTACTTCGATCTCTGGGGCCGTG
GCACCCTGGTCACCGTCTCGAGCGGTGGAGGCGGTTCAGGCGGAGGTGGTTCCGGCGGTGGCGGC
TCCCAGTCTGCCCTGACTCAGCCTGCCTCCGTGTCTGGGTCTCCTGGACAGTCGATCACCATCTCCTG
CACTGGAACCAGCAGTGACGTTGGTGGTTATAACTTTGTCTCCTGGTACCAACAACACCCAGGCAAA
GCCCCCAAACTCATGATCTATGATGTCAGTGATCGGCCCTCAGGGGTGTCTGATCGCTTCTCCGGCTC
CAAGTCTGGCAACACGGCCTCCCTGATCATCTCTGGCCTCCAGGCTGACGACGAGGCTGATTATTACT
GCAGCTCATATGGGAGCAGCAGCACTCATGTGATTTTCGGCGGAGGGACCAAGGTGACCGTCCTA
SI-1X2 BISPECIFIC HEAVY CHAIN FULL-LENGTH AMINO ACID SEQUENCE (SEQ ID NO: 164).
HUMAN GAMMA-1 DOMAIN IS UNDERLINED (SEQ ID NO: 238), CONNECTOR IS IN ITALICS
(SEQ ID NO: 239), SCFV IS IN BOLD (SEQ ID NO: 240)
QVQLQESGPGLVKPSETLSLTCTVSGGSVSSGDYYWTWIRQSPGKGLEWIGHIYYSGNTNYNPSLKSRLTI
SIDTSKTQFSLKLSSVTAADTAIYYCVRDRVTGAFDIWGQGTMVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGGGGSQVQLQESGGGLVKPGGSLRLSCAASGFTFSS
YWMSWVRQAPGKGLEWVANINRDGSASYYVDSVKGRFTISRDDAKNSLYLQMNSLRAEDTAVYYC
ARDRGVGYFDLWGRGTLVTVSSGGGGSGGGGSGGGGSQSALTQPASVSGSPGQSITISCTGTSSDVG
GYNFVSWYQQHPGKAPKLMIYDVSDRPSGVSDRFSGSKSGNTASLIISGLQADDEADYYCSSYGSSST
HVIFGGGTKVTVL
SI-1X2 BISPECIFIC HEAVY CHAIN VARIABLE HEAVY CHAIN AMINO ACID SEQUENCE (SEQ ID
NO: 165). COMPLEMENTARITY DETERMINING REGIONS ARE UNDERLINED (SEQ ID NOS: 241,
242 AND 243)
QVQLQESGPGLVKPSETLSLTCTVSGGSVSSGDYYWTWIRQSPGKGLEWIGHIYYSGNTNYNPSLKSRLTI
SIDTSKTQFSLKLSSVTAADTAIYYCVRDRVTGAFDIWGQGTMVTVSS
SI1X2 BISPECIFIC HEAVY CHAIN SCFV AMINO ACID SEQUENCE (SEQ ID NO: 166). ORDER: VH
(SEQ ID NO: 244)-LINKER (SEQ ID NO: 245)-VL (SEQ ID NO: 246). COMPLEMENTARITY
DETERMINING REGIONS ARE UNDERLINED (SEQ ID NOS: 247, 248, 249, 250, 251 AND 252).
LINKER IS IN BOLD ITALICS (SEQ ID NO: 245)
QVQLQESGGGLVKPGGSLRLSCAASGFTFSSYWMSWVRQAPGKGLEWVANINRDGSASYYVDSVKGR
FTISRDDAKNSLYLQMNSLRAEDTAVYYCARDRGVGYFDLWGRGTLVTVSSGGGGGGGGSGGGGSQ
SALTQPASVSGSPGQSITISCTGTSSDVGGYNFVSWYQQHPGKAPKLMIYDVSDRPSGVSDRFSGSKSGN
TASLIISGLQADDEADYYCSSYGSSSTHVIFGGGTKVTVL
SI-1X3 SEQUENCES
SI1X3 LIGHT CHAIN FULL-LENGTH NUCLEOTIDE SEQUENCE (SEQ ID NO: 167)
CAGTCTGCCCTGACTCAGCCTGCCTCCGTGTCTGGGTCTCCTGGACAGTCGATCACCATCTCCTGCAC
TGGAACCAGCAGTGACGTTGGTGGTTATAACTTTGTCTCCTGGTACCAACAACACCCAGGCAAAGCC
CCCAAACTCATGATCTATGATGTCAGTGATCGGCCCTCAGGGGTGTCTGATCGCTTCTCCGGCTCCAA
GTCTGGCAACACGGCCTCCCTGATCATCTCTGGCCTCCAGGCTGACGACGAGGCTGATTATTACTGC
AGCTCATATGGGAGCAGCAGCACTCATGTGATTTTCGGCGGAGGGACCAAGCTGACCGTCCTACGT
ACGGTGGCTGCACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGT
SI-1X3 LIGHT CHAIN VARIABLE LIGHT CHAIN NUCLEOTIDE SEQUENCE (SEQ ID NO: 168)
CAGTCTGCCCTGACTCAGCCTGCCTCCGTGTCTGGGTCTCCTGGACAGTCGATCACCATCTCCTGCAC
TGGAACCAGCAGTGACGTTGGTGGTTATAACTTTGTCTCCTGGTACCAACAACACCCAGGCAAAGCC
CCCAAACTCATGATCTATGATGTCAGTGATCGGCCCTCAGGGGTGTCTGATCGCTTCTCCGGCTCCAA
GTCTGGCAACACGGCCTCCCTGATCATCTCTGGCCTCCAGGCTGACGACGAGGCTGATTATTACTGC
AGCTCATATGGGAGCAGCAGCACTCATGTGATTTTCGGCGGAGGGACCAAGCTGACCGTCCTA
SI-1X3 LIGHT CHAIN FULL-LENGTH AMINO ACID SEQUENCE (SEQ ID NO: 169). HUMAN
KAPPA CONSTANT DOMAIN IS UNDERLINED (SEQ ID NO: 253)
QSALTQPASVSGSPGQSITISCTGTSSDVGGYNFVSWYQQHPGKAPKLMIYDVSDRPSGVSDRFSGSKSG
NTASLIISGLQADDEADYYCSSYGSSSTHVIFGGGTKLTVLRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKSF
NRGEC
SI-1X3 LIGHT CHAIN VARIABLE LIGHT CHAIN AMINO ACID SEQUENCE (SEQ ID NO: 170).
COMPLEMENTARITY DETERMINING REGIONS ARE UNDERLINED (SEQ ID NOS: 254, 255 AND 256)
QSALTQPASVSGSPGQSITISCTGTSSDVGGYNFVSWYQQHPGKAPKLMIYDVSDRPSGVSDRFSGSKSG
NTASLIISGLQADDEADYYCSSYGSSSTHVIFGGGTKLTVL
SI1X3 BISPECIFIC HEAVY CHAIN FULL-LENGTH NUCLEOTIDE SEQUENCE (SEQ ID NO: 171)
CAGGTGCAGCTGCAGGAGTCGGGGGGAGGCCTGGTCAAGCCTGGAGGGTCCCTGAGACTCTCCTGT
GCAGCCTCTGGATTCACCTTTAGTAGTTATTGGATGAGCTGGGTCCGCCAGGCTCCAGGGAAGGGG
CTGGAGTGGGTGGCCAACATAAACCGCGATGGAAGTGCGAGTTACTATGTGGACTCTGTGAAGGGC
CGATTCACCATCTCCAGAGACGACGCCAAGAACTCACTGTATCTGCAAATGAACAGCCTGAGAGCTG
AGGACACGGCTGTGTATTACTGTGCGAGAGATCGTGGGGTGGGCTACTTCGATCTCTGGGGCCGTG
GCACCCTGGTCACCGTCTCGAGCGCTAGCACCAAGGGCCCATCGGTCTTCCCCCTGGCACCCTCCTCC
AAGAGCACCTCTGGGGGCACAGCGGCCCTGGGCTGCCTGGTCAAGGACTACTTCCCCGAACCGGTG
ACGGTGTCGTGGAACTCAGGCGCCCTGACCAGCGGCGTGCACACCTTCCCGGCTGTCCTACAGTCCT
CAGGACTCTACTCCCTCAGCAGCGTGGTGACCGTGCCCTCCAGCAGCTTGGGCACCCAGACCTACAT
CTGCAACGTGAATCACAAGCCCAGCAACACCAAGGTGGACAAGAGAGTTGAGCCCAAATCTTGTGA
CAAAACTCACACATGCCCACCGTGCCCAGCACCTGAACTCCTGGGGGGACCGTCAGTCTTCCTCTTCC
CCCCAAAACCCAAGGACACCCTCATGATCTCCCGGACCCCTGAGGTCACATGCGTGGTGGTGGACGT
GAGCCACGAAGACCCTGAGGTCAAGTTCAACTGGTACGTGGACGGCGTGGAGGTGCATAATGCCAA
GACAAAGCCGCGGGAGGAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTACACCCTGCCCCCATCCC
GGGAGGAGATGACCAAGAACCAGGTCAGCCTGACCTGCCTGGTCAAAGGCTTCTATCCCAGCGACA
TCGCCGTGGAGTGGGAGAGCAATGGGCAGCCGGAGAACAACTACAAGACCACGCCTCCCGTGCTG
GACTCCGACGGCTCCTTCTTCCTCTATAGCAAGCTCACCGTGGACAAGAGCAGGTGGCAGCAGGGG
AACGTCTTCTCATGCTCCGTGATGCATGAGGCTCTGCACAACCACTACACGCAGAAGAGCCTCTCCCT
GTCTCCGGGTAAAGGCGGTGGAGGATCCGGCGGTGGTGGATCACAAGTTCAACTTCAACAATCTGG
TGCTGAAGTTAAAAAACCTGGTTCTTCTGTTAAAGTTTCTTGTAAAGCCTCTGGTTATACTTTTACTAA
TTATTATATTTATTGGGTTCGTCAAGCTCCTGGTCAAGGTCTTGAATGGATTGGTGGTATTAATCCTAC
TTCTGGTGGTTCTAATTTTAATGAAAAATTTAAAACTCGTGTTACTATTACTGTTGATGAATCTACGAA
CACTGCTTATATGGAACTTTCTTCTCTTCGTTCTGAAGATACTGCTTTTTATTTTTGTGCGCGTCAAGGT
CTTTGGTTTGATTCTGATGGTCGTGGTTTTGATTTTTGGGGTCAAGGTTCCACTGTTACTGTCTCGAGC
GGTGGAGGCGGTTCAGGCGGAGGTGGTTCCGGCGGTGGCGGCTCCGATATTCAAATGACTCAATCT
CCTTCTTCTCTTTCTGCTTCTGTTGGTGATCGTGTTACTATTACTTGTCGTTCTTCTCAAAATATTGTTCA
TTCTAATGGTAATACTTATCTTGATTGGTATCAACAAACTCCTGGTAAAGCTCCTAAACTTCTTATTTA
TAAAGTTTCTAATCGTTTTTCTGGTGTTCCTTCTCGTTTTTCTGGTTCTGGTTCTGGTACTGATTTTACTT
TTACTATTTCTTCTCTTCAACCTGAAGATATTGCTACTTATTATTGTTTTCAATATTCTCATGTTCCTTGG
ACTTTTGGTCAAGGTACTAAACTTCAAATTACTCGT
SI-1X3 BISPECIFIC HEAVY CHAIN VARIABLE HEAVY CHAIN NUCLEOTIDE SEQUENCE (SEQ ID
NO: 172)
CAGGTGCAGCTGCAGGAGTCGGGGGGAGGCCTGGTCAAGCCTGGAGGGTCCCTGAGACTCTCCTGT
GCAGCCTCTGGATTCACCTTTAGTAGTTATTGGATGAGCTGGGTCCGCCAGGCTCCAGGGAAGGGG
CTGGAGTGGGTGGCCAACATAAACCGCGATGGAAGTGCGAGTTACTATGTGGACTCTGTGAAGGGC
CGATTCACCATCTCCAGAGACGACGCCAAGAACTCACTGTATCTGCAAATGAACAGCCTGAGAGCTG
AGGACACGGCTGTGTATTACTGTGCGAGAGATCGTGGGGTGGGCTACTTCGATCTCTGGGGCCGTG
GCACCCTGGTCACCGTCTCGAGC
SI-1X3 BISPECIFIC HEAVY CHAIN SCFV NUCLEOTIDE SEQUENCE (SEQ ID NO: 173)
CAAGTTCAACTTCAACAATCTGGTGCTGAAGTTAAAAAACCTGGTTCTTCTGTTAAAGTTTCTTGTAA
AGCCTCTGGTTATACTTTTACTAATTATTATATTTATTGGGTTCGTCAAGCTCCTGGTCAAGGTCTTGA
ATGGATTGGTGGTATTAATCCTACTTCTGGTGGTTCTAATTTTAATGAAAAATTTAAAACTCGTGTTAC
TATTACTGTTGATGAATCTACGAACACTGCTTATATGGAACTTTCTTCTCTTCGTTCTGAAGATACTGC
TTTTTATTTTTGTGCGCGTCAAGGTCTTTGGTTTGATTCTGATGGTCGTGGTTTTGATTTTTGGGGTCA
AGGTTCCACTGTTACTGTCTCGAGCGGTGGAGGCGGTTCAGGCGGAGGTGGTTCCGGCGGTGGCGG
CTCCGATATTCAAATGACTCAATCTCCTTCTTCTCTTTCTGCTTCTGTTGGTGATCGTGTTACTATTACT
TGTCGTTCTTCTCAAAATATTGTTCATTCTAATGGTAATACTTATCTTGATTGGTATCAACAAACTCCTG
GTAAAGCTCCTAAACTTCTTATTTATAAAGTTTCTAATCGTTTTTCTGGTGTTCCTTCTCGTTTTTCTGG
TTCTGGTTCTGGTACTGATTTTACTTTTACTATTTCTTCTCTTCAACCTGAAGATATTGCTACTTATTATT
GTTTTCAATATTCTCATGTTCCTTGGACTTTTGGTCAAGGTACTAAACTTCAAATTACTCGT
SI-1X3 BISPECIFIC HEAVY CHAIN FULL-LENGTH AMINO ACID SEQUENCE (SEQ ID NO: 174).
HUMAN GAMMA-1 DOMAIN IS UNDERLINED (SEQ ID NO: 257), CONNECTOR IS IN ITALICS
(SEQ ID NO: 258), SCFV IS IN BOLD (SEQ ID NO: 259)
QVQLQESGGGLVKPGGSLRLSCAASGFTFSSYWMSWVRQAPGKGLEWVANINRDGSASYYVDSVKGR
FTISRDDAKNSLYLQMNSLRAEDTAVYYCARDRGVGYFDLWGRGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPS
NTKVDKRVEPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGKGGGGSGGGGSQVQLQQSGAEVKKPGSSVKVSCKASG
YTFTNYYIYWVRQAPGQGLEWIGGINPTSGGSNFNEKFKTRVTITVDESTNTAYMELSSLRSEDTAFYF
CARQGLWFDSDGRGFDFWGQGSTVTVSSGGGGSGGGGSGGGGSDIQMTQSPSSLSASVGDRVTIT
CRSSQNIVHSNGNTYLDWYQQTPGKAPKLLIYKVSNRFSGVPSRFSGSGSGTDFTFTISSLQPEDIATYY
CFQYSHVPWTFGQGTKLQITR
SI-1X3 BISPECIFIC HEAVY CHAIN VARIABLE HEAVY CHAIN AMINO ACID SEQUENCE (SEQ ID
NO: 175). COMPLEMENTARITY DETERMINING REGIONS ARE UNDERLINED (SEQ ID NOS: 260,
261 AND 262)
QVQLQESGGGLVKPGGSLRLSCAASGFTFSSYWMSWVRQAPGKGLEWVANINRDGSASYYVDSVKGR
FTISRDDAKNSLYLQMNSLRAEDTAVYYCARDRGVGYFDLWGRGTLVTVSS
SI1X3 BISPECIFIC HEAVY CHAIN SCFV AMINO ACID SEQUENCE (SEQ ID NO: 176). ORDER: VH
(SEQ ID NO: 263)-LINKER (SEQ ID NO: 264)-VL (SEQ ID NO: 265). COMPLEMENTARITY
DETERMINING REGIONS ARE UNDERLINED (SEQ ID NOS: 266, 267, 268, 269, 270 AND 271).
LINKER IS IN BOLD ITALICS (SEQ ID NO: 264)
QVQLQQSGAEVKKPGSSVKVSCKASGYTFTNYYIYWVRQAPGQGLEWIGGINPTSGGSNFNEKFKTRVT
ITVDESTNTAYMELSSLRSEDTAFYFCARQGLWFDSDGRGFDFWGQGSTVTVSSGGGGSGGGGSGGG
GSDIQMTQSPSSLSASVGDRVTITCRSSQNIVHSNGNTYLDWYQQTPGKAPKLLIYKVSNRFSGVPSRFS
GSGSGTDFTFTISSLQPEDIATYYCFQYSHVPWTFGQGTKLQITR
SI-1X4 SEQUENCES
SI1X4 LIGHT CHAIN FULL-LENGTH NUCLEOTIDE SEQUENCE (SEQ ID NO: 177)
GATATTCAAATGACTCAATCTCCTTCTTCTCTTTCTGCTTCTGTTGGTGATCGTGTTACTATTACTTGTC
GTTCTTCTCAAAATATTGTTCATTCTAATGGTAATACTTATCTTGATTGGTATCAACAAACTCCTGGTA
AAGCTCCTAAACTTCTTATTTATAAAGTTTCTAATCGTTTTTCTGGTGTTCCTTCTCGTTTTTCTGGTTCT
GGTTCTGGTACTGATTTTACTTTTACTATTTCTTCTCTTCAACCTGAAGATATTGCTACTTATTATTGTTT
TCAATATTCTCATGTTCCTTGGACTTTTGGTCAAGGTACTAAACTTCAAATTACTCGTACGGTGGCTGC
ACCATCTGTCTTCATCTTCCCGCCATCTGATGAGCAGTTGAAATCTGGAACTGCCTCTGTTGTGTGCCT
GCTGAATAACTTCTATCCCAGAGAGGCCAAAGTACAGTGGAAGGTGGATAACGCCCTCCAATCGGG
TAACTCCCAGGAGAGTGTCACAGAGCAGGACAGCAAGGACAGCACCTACAGCCTCAGCAGCACCCT
GACGCTGAGCAAAGCAGACTACGAGAAACACAAAGTCTACGCCTGCGAAGTCACCCATCAGGGCCT
GAGCTCGCCCGTCACAAAGAGCTTCAACAGGGGAGAGTGT
SI-1X4 LIGHT CHAIN VARIABLE LIGHT CHAIN NUCLEOTIDE SEQUENCE (SEQ ID NO: 178)
GATATTCAAATGACTCAATCTCCTTCTTCTCTTTCTGCTTCTGTTGGTGATCGTGTTACTATTACTTGTC
GTTCTTCTCAAAATATTGTTCATTCTAATGGTAATACTTATCTTGATTGGTATCAACAAACTCCTGGTA
AAGCTCCTAAACTTCTTATTTATAAAGTTTCTAATCGTTTTTCTGGTGTTCCTTCTCGTTTTTCTGGTTCT
  GGTTCTGGTACTGATTTTACTTTTACTATTTCTTCTCTTCAACCTGAAGATATTGCTACTTATTATTGTTT
TCAATATTCTCATGTTCCTTGGACTTTTGGTCAAGGTACTAAACTTCAAATTACT
SI-1X4 LIGHT CHAIN FULL-LENGTH AMINO ACID SEQUENCE (SEQ ID NO: 179). HUMAN
KAPPA CONSTANT DOMAIN IS UNDERLINED (SEQ ID NO: 272)
DIQMTQSPSSLSASVGDRVTITCRSSQNIVHSNGNTYLDWYQQTPGKAPKLLIYKVSNRFSGVPSRFSGSG
SGTDFTFTISSLQPEDIATYYCFQYSHVPWTFGQGTKLQITRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKSF
NRGEC
SI-1X4 LIGHT CHAIN VARIABLE LIGHT CHAIN AMINO ACID SEQUENCE (SEQ ID NO: 180).
COMPLEMENTARITY DETERMINING REGIONS ARE UNDERLINED (SEQ ID NOS: 273, 274 AND 275)
DIQMTQSPSSLSASVGDRVTITCRSSQNIVHSNGNTYLDWYQQTPGKAPKLLIYKVSNRFSGVPSRFSGSG
SGTDFTFTISSLQPEDIATYYCFQYSHVPWTFGQGTKLQIT
SI1X4 BISPECIFIC HEAVY CHAIN FULL-LENGTH NUCLEOTIDE SEQUENCE (SEQ ID NO: 181)
CAAGTTCAACTTCAACAATCTGGTGCTGAAGTTAAAAAACCTGGTTCTTCTGTTAAAGTTTCTTGTAA
AGCCTCTGGTTATACTTTTACTAATTATTATATTTATTGGGTTCGTCAAGCTCCTGGTCAAGGTCTTGA
ATGGATTGGTGGTATTAATCCTACTTCTGGTGGTTCTAATTTTAATGAAAAATTTAAAACTCGTGTTAC
TATTACTGTTGATGAATCTACGAACACTGCTTATATGGAACTTTCTTCTCTTCGTTCTGAAGATACTGC
TTTTTATTTTTGTGCGCGTCAAGGTCTTTGGTTTGATTCTGATGGTCGTGGTTTTGATTTTTGGGGTCA
AGGTTCCACTGTTACTGTCTCGAGCGCTAGCACCAAGGGCCCATCGGTCTTCCCCCTGGCACCCTCCT
CCAAGAGCACCTCTGGGGGCACAGCGGCCCTGGGCTGCCTGGTCAAGGACTACTTCCCCGAACCGG
TGACGGTGTCGTGGAACTCAGGCGCCCTGACCAGCGGCGTGCACACCTTCCCGGCTGTCCTACAGTC
CTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGT
GACAAAACTCACACATGCCCACCGTGCCCAGCACCTGAACTCCTGGGGGGACCGTCAGTCTTCCTCTT
CCCCCCAAAACCCAAGGACACCCTCATGATCTCCCGGACCCCTGAGGTCACATGCGTGGTGGTGGAC
GTGAGCCACGAAGACCCTGAGGTCAAGTTCAACTGGTACGTGGACGGCGTGGAGGTGCATAATGCC
AAGACAAAGCCGCGGGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTGTACACCCTGCCCCCAT
CCCGGGAGGAGATGACCAAGAACCAGGTCAGCCTGACCTGCCTGGTCAAAGGCTTCTATCCCAGCG
ACATCGCCGTGGAGTGGGAGAGCAATGGGCAGCCGGAGAACAACTACAAGACCACGCCTCCCGTGC
TGGACTCCGACGGCTCCTTCTTCCTCTATAGCAAGCTCACCGTGGACAAGAGCAGGTGGCAGCAGGG
GAACGTCTTCTCATGCTCCGTGATGCATGAGGCTCTGCACAACCACTACACGCAGAAGAGCCTCTCCC
TGTCTCCGGGTAAAGGCGGTGGAGGATCCGGCGGTGGTGGATCACAGGTGCAGCTGCAGGAGTCG
GGGGGAGGCCTGGTCAAGCCTGGAGGGTCCCTGAGACTCTCCTGTGCAGCCTCTGGATTCACCTTTA
GTAGTTATTGGATGAGCTGGGTCCGCCAGGCTCCAGGGAAGGGGCTGGAGTGGGTGGCCAACATA
AACCGCGATGGAAGTGCGAGTTACTATGTGGACTCTGTGAAGGGCCGATTCACCATCTCCAGAGAC
GACGCCAAGAACTCACTGTATCTGCAAATGAACAGCCTGAGAGCTGAGGACACGGCTGTGTATTACT
GTGCGAGAGATCGTGGGGTGGGCTACTTCGATCTCTGGGGCCGTGGCACCCTGGTCACCGTCTCGA
GCGGTGGAGGCGGTTCAGGCGGAGGTGGTTCCGGCGGTGGCGGCTCCCAGTCTGCCCTGACTCAGC
CTGCCTCCGTGTCTGGGTCTCCTGGACAGTCGATCACCATCTCCTGCACTGGAACCAGCAGTGACGTT
GGTGGTTATAACTTTGTCTCCTGGTACCAACAACACCCAGGCAAAGCCCCCAAACTCATGATCTATGA
TGTCAGTGATCGGCCCTCAGGGGTGTCTGATCGCTTCTCCGGCTCCAAGTCTGGCAACACGGCCTCC
  CTGATCATCTCTGGCCTCCAGGCTGACGACGAGGCTGATTATTACTGCAGCTCATATGGGAGCAGCA
GCACTCATGTGATTITCGGCGGAGGGACCAAGGTGACCGTCCTA
SI-1X4 BISPECIFIC HEAVY CHAIN VARIABLE HEAVY CHAIN NUCLEOTIDE SEQUENCE (SEQ ID
NO: 182)
CAAGTTCAACTTCAACAATCTGGTGCTGAAGTTAAAAAACCTGGTTCTTCTGTTAAAGTTTCTTGTAA
AGCCTCTGGTTATACTTTTACTAATTATTATATTTATTGGGTTCGTCAAGCTCCTGGTCAAGGTCTTGA
ATGGATTGGTGGTATTAATCCTACTTCTGGTGGTTCTAATTTTAATGAAAAATTTAAAACTCGTGTTAC
TATTACTGTTGATGAATCTACGAACACTGCTTATATGGAACTTTCTTCTCTTCGTTCTGAAGATACTGC
TTTTTATTTTTGTGCGCGTCAAGGTCTTTGGTTTGATTCTGATGGTCGTGGTTTTGATTTTTGGGGTCA
AGGTTCCACTGTTACTGTCTCGAGC
SI-1X4 BISPECIFIC HEAVY CHAIN SCFV NUCLEOTIDE SEQUENCE (SEQ ID NO: 183)
CAGGTGCAGCTGCAGGAGTCGGGGGGAGGCCTGGTCAAGCCTGGAGGGTCCCTGAGACTCTCCTGT
GCAGCCTCTGGATTCACCTTTAGTAGTTATTGGATGAGCTGGGTCCGCCAGGCTCCAGGGAAGGGG
CTGGAGTGGGTGGCCAACATAAACCGCGATGGAAGTGCGAGTTACTATGTGGACTCTGTGAAGGGC
CGATTCACCATCTCCAGAGACGACGCCAAGAACTCACTGTATCTGCAAATGAACAGCCTGAGAGCTG
AGGACACGGCTGTGTATTACTGTGCGAGAGATCGTGGGGTGGGCTACTTCGATCTCTGGGGCCGTG
GCACCCTGGTCACCGTCTCGAGCGGTGGAGGCGGTTCAGGCGGAGGTGGTTCCGGCGGTGGCGGC
TCCCAGTCTGCCCTGACTCAGCCTGCCTCCGTGTCTGGGTCTCCTGGACAGTCGATCACCATCTCCTG
CACTGGAACCAGCAGTGACGTTGGTGGTTATAACTTTGTCTCCTGGTACCAACAACACCCAGGCAAA
GCCCCCAAACTCATGATCTATGATGTCAGTGATCGGCCCTCAGGGGTGTCTGATCGCTTCTCCGGCTC
CAAGTCTGGCAACACGGCCTCCCTGATCATCTCTGGCCTCCAGGCTGACGACGAGGCTGATTATTACT
GCAGCTCATATGGGAGCAGCAGCACTCATGTGATTTTCGGCGGAGGGACCAAGGTGACCGTCCTA
SI-1X4 BISPECIFIC HEAVY CHAIN FULL-LENGTH AMINO ACID SEQUENCE (SEQ ID NO: 184).
HUMAN GAMMA-1 DOMAIN IS UNDERLINED (SEQ ID NO: 276), CONNECTOR IS IN ITALICS
(SEQ ID NO: 277), SCFV IS IN BOLD (SEQ ID NO: 278)
QVQLQQSGAEVKKPGSSVKVSCKASGYTFTNYYIYWVRQAPGQGLEWIGGINPTSGGSNFNEKFKTRVT
ITVDESTNTAYMELSSLRSEDTAFYFCARQGLWFDSDGRGFDFWGQGSTVTVSSASTKGPSVFPLAPSSK
STSGGTAALGCLVKDYFPEPVTVSWNSGALTSGVHTFPAVLQSSGLYSLSSVVTVPSSSLGTQTYICNVNH
KPSNTKVDKRVEPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGKGGGGSGGGGSQVQLQESGGGLVKPGGSLRLSCAA
SGFTFSSYWMSWVRQAPGKGLEWVANINRDGSASYYVDSVKGRFTISRDDAKNSLYLQMNSLRAED
TAVYYCARDRGVGYFDLWGRGTLVTVSSGGGGSGGGGSGGGGSQSALTQPASVSGSPGQSITISCTG
TSSDVGGYNFVSWYQQHPGKAPKLMIYDVSDRPSGVSDRFSGSKSGNTASLIISGLQADDEADYYCSS
YGSSSTHVIFGGGTKVTVL
SI-1X4 BISPECIFIC HEAVY CHAIN VARIABLE HEAVY CHAIN AMINO ACID SEQUENCE (SEQ ID
NO: 185). COMPLEMENTARITY DETERMINING REGIONS ARE UNDERLINED (SEQ ID NOS: 279,
280 AND 281)
QVQLQQSGAEVKKPGSSVKVSCKASGYTFTNYYIYWVRQAPGQGLEWIGGINPTSGGSNFNEKFKTRVT
ITVDESTNTAYMELSSLRSEDTAFYFCARQGLWFDSDGRGFDFWGQGSTVTVSS
SI1X4 BISPECIFIC HEAVY CHAIN SCFV AMINO ACID SEQUENCE (SEQ ID NO: 186). ORDER: VH
(SEQ ID NO: 282)-LINKER (SEQ ID NO: 283)-VL (SEQ ID NO: 284). COMPLEMENTARITY
DETERMINING REGIONS ARE UNDERLINED (SEQ ID NOS: 285, 286, 287, 288, 289 AND 290).
LINKER IS IN BOLD ITALICS (SEQ ID NO: 283)
QVQLQESGGGLVKPGGSLRLSCAASGFTFSSYWMSWVRQAPGKGLEWVANINRDGSASYYVDSVKGR
FTISRDDAKNSLYLQMNSLRAEDTAVYYCARDRGVGYFDLWGRGTLVTVSSGGGGSGGGGSGGGGSQ
SALTQPASVSGSPGQSITISCTGTSSDVGGYNFVSWYQQHPGKAPKLMIYDVSDRPSGVSDRFSGSKSGN
TASLIISGLQADDEADYYCSSYGSSSTHVIFGGGTKVTVL
SI-1X5 SEQUENCES
SI1X5 LIGHT CHAIN FULL-LENGTH NUCLEOTIDE SEQUENCE (SEQ ID NO: 187)
CAGTCTGCCCTGACTCAGCCTGCCTCCGTGTCTGGGTCTCCTGGACAGTCGATCACCATCTCCTGCAC
TGGAACCAGCAGTGACGTTGGTGGTTATAACTTTGTCTCCTGGTACCAACAACACCCAGGCAAAGCC
CCCAAACTCATGATCTATGATGTCAGTGATCGGCCCTCAGGGGTGTCTGATCGCTTCTCCGGCTCCAA
GTCTGGCAACACGGCCTCCCTGATCATCTCTGGCCTCCAGGCTGACGACGAGGCTGATTATTACTGC
AGCTCATATGGGAGCAGCAGCACTCATGTGATTTTCGGCGGAGGGACCAAGCTGACCGTCCTACGT
ACGGTGGCTGCACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGT
SI-1X5 LIGHT CHAIN VARIABLE LIGHT CHAIN NUCLEOTIDE SEQUENCE (SEQ ID NO: 188)
CAGTCTGCCCTGACTCAGCCTGCCTCCGTGTCTGGGTCTCCTGGACAGTCGATCACCATCTCCTGCAC
TGGAACCAGCAGTGACGTTGGTGGTTATAACTTTGTCTCCTGGTACCAACAACACCCAGGCAAAGCC
CCCAAACTCATGATCTATGATGTCAGTGATCGGCCCTCAGGGGTGTCTGATCGCTTCTCCGGCTCCAA
GTCTGGCAACACGGCCTCCCTGATCATCTCTGGCCTCCAGGCTGACGACGAGGCTGATTATTACTGC
AGCTCATATGGGAGCAGCAGCACTCATGTGATTTTCGGCGGAGGGACCAAGCTGACCGTCCTA
SI-1X5 LIGHT CHAIN FULL-LENGTH AMINO ACID SEQUENCE (SEQ ID NO: 189). HUMAN
KAPPA CONSTANT DOMAIN IS UNDERLINED (SEQ ID NO: 291)
QSALTQPASVSGSPGQSITISCTGTSSDVGGYNFVSWYQQHPGKAPKLMIYDVSDRPSGVSDRFSGSKSG
NTASLIISGLQADDEADYYCSSYGSSSTHVIFGGGTKLTVLRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKSF
NRGEC
SI-1X5 LIGHT CHAIN VARIABLE LIGHT CHAIN AMINO ACID SEQUENCE (SEQ ID NO: 190).
COMPLEMENTARITY DETERMINING REGIONS ARE UNDERLINED (SEQ ID NOS: 292, 293 AND 294)
QSALTQPASVSGSPGQSITISCTGTSSDVGGYNFVSWYQQHPGKAPKLMIYDVSDRPSGVSDRFSGSKSG
NTASLIISGLQADDEADYYCSSYGSSSTHVIFGGGTKLTVL
SI1X5 BISPECIFIC HEAVY CHAIN FULL-LENGTH NUCLEOTIDE SEQUENCE (SEQ ID NO: 191)
CAGGTGCAGCTGCAGGAGTCGGGGGGAGGCCTGGTCAAGCCTGGAGGGTCCCTGAGACTCTCCTGT
GCAGCCTCTGGATTCACCTTTAGTAGTTATTGGATGAGCTGGGTCCGCCAGGCTCCAGGGAAGGGG
CTGGAGTGGGTGGCCAACATAAACCGCGATGGAAGTGCGAGTTACTATGTGGACTCTGTGAAGGGC
CGATTCACCATCTCCAGAGACGACGCCAAGAACTCACTGTATCTGCAAATGAACAGCCTGAGAGCTG
AGGACACGGCTGTGTATTACTGTGCGAGAGATCGTGGGGTGGGCTACTTCGATCTCTGGGGCCGTG
GCACCCTGGTCACCGTCTCGAGCGCTAGCACCAAGGGCCCATCGGTCTTCCCCCTGGCACCCTCCTCC
AAGAGCACCTCTGGGGGCACAGCGGCCCTGGGCTGCCTGGTCAAGGACTACTTCCCCGAACCGGTG
ACGGTGTCGTGGAACTCAGGCGCCCTGACCAGCGGCGTGCACACCTTCCCGGCTGTCCTACAGTCCT
CAGGACTCTACTCCCTCAGCAGCGTGGTGACCGTGCCCTCCAGCAGCTTGGGCACCCAGACCTACAT
CTGCAACGTGAATCACAAGCCCAGCAACACCAAGGTGGACAAGAGAGTTGAGCCCAAATCTTGTGA
CAAAACTCACACATGCCCACCGTGCCCAGCACCTGAACTCCTGGGGGGACCGTCAGTCTTCCTCTTCC
CCCCAAAACCCAAGGACACCCTCATGATCTCCCGGACCCCTGAGGTCACATGCGTGGTGGTGGACGT
GAGCCACGAAGACCCTGAGGTCAAGTTCAACTGGTACGTGGACGGCGTGGAGGTGCATAATGCCAA
GACAAAGCCGCGGGAGGAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTACACCCTGCCCCCATCCC
GGGAGGAGATGACCAAGAACCAGGTCAGCCTGACCTGCCTGGTCAAAGGCTTCTATCCCAGCGACA
TCGCCGTGGAGTGGGAGAGCAATGGGCAGCCGGAGAACAACTACAAGACCACGCCTCCCGTGCTG
GACTCCGACGGCTCCTTCTTCCTCTATAGCAAGCTCACCGTGGACAAGAGCAGGTGGCAGCAGGGG
AACGTCTTCTCATGCTCCGTGATGCATGAGGCTCTGCACAACCACTACACGCAGAAGAGCCTCTCCCT
GTCTCCGGGTAAAGGCGGTGGAGGATCCGGCGGTGGTGGATCACAGGTGCAGCTGAAGCAGTCAG
GACCTGGCCTAGTGCAGCCCTCACAGAGCCTGTCCATCACCTGCACAGTCTCTGGTTTCTCATTAACT
AACTATGGTGTACACTGGGTTCGCCAGTCTCCAGGAAAGGGTCTGGAGTGGCTGGGAGTGATATGG
AGTGGTGGAAACACAGACTATAATACACCTTTCACATCCAGACTGAGCATCAACAAGGACAATTCCA
AGAGCCAAGTTTTCTTTAAAATGAACAGTCTGCAATCTAATGACACAGCCATATATTACTGTGCCAGA
GCCCTCACCTACTATGATTACGAGTTTGCTTACTGGGGCCAAGGGACTCTGGTCACTGTCTCTAGCGG
TGGAGGCGGTTCAGGCGGAGGTGGTTCCGGCGGTGGCGGCTCCGACATCTTGCTGACTCAGTCTCC
AGTCATCCTGTCTGTGAGTCCAGGAGAAAGAGTCAGTTTCTCCTGCAGGGCCAGTCAGAGTATTGGC
ACAAACATACACTGGTATCAGCAAAGAACAAATGGTTCTCCAAGGCTTCTCATAAAGTATGCTTCTGA
GTCTATCTCTGGGATTCCTTCCAGGTTTAGTGGCAGTGGATCAGGGACAGATTITACTCTTAGCATCA
ACAGTGTGGAGTCTGAAGATATTGCAGATTATTACTGTCAACAAAATAATAACTGGCCAACCACGTT
CGGTGCTGGGACCAAGCTGGAGCTGAAACGT
SI-1X5 BISPECIFIC HEAVY CHAIN VARIABLE HEAVY CHAIN NUCLEOTIDE SEQUENCE (SEQ ID
NO: 192)
CAGGTGCAGCTGCAGGAGTCGGGGGGAGGCCTGGTCAAGCCTGGAGGGTCCCTGAGACTCTCCTGT
GCAGCCTCTGGATTCACCTTTAGTAGTTATTGGATGAGCTGGGTCCGCCAGGCTCCAGGGAAGGGG
CTGGAGTGGGTGGCCAACATAAACCGCGATGGAAGTGCGAGTTACTATGTGGACTCTGTGAAGGGC
CGATTCACCATCTCCAGAGACGACGCCAAGAACTCACTGTATCTGCAAATGAACAGCCTGAGAGCTG
AGGACACGGCTGTGTATTACTGTGCGAGAGATCGTGGGGTGGGCTACTTCGATCTCTGGGGCCGTG
GCACCCTGGTCACCGTCTCGAGC
SI-1X5 BISPECIFIC HEAVY CHAIN SCFV NUCLEOTIDE SEQUENCE (SEQ ID NO: 193)
CAGGTGCAGCTGAAGCAGTCAGGACCTGGCCTAGTGCAGCCCTCACAGAGCCTGTCCATCACCTGCA
CAGTCTCTGGTTTCTCATTAACTAACTATGGTGTACACTGGGTTCGCCAGTCTCCAGGAAAGGGTCTG
GAGTGGCTGGGAGTGATATGGAGTGGTGGAAACACAGACTATAATACACCTTTCACATCCAGACTG
AGCATCAACAAGGACAATTCCAAGAGCCAAGTTTTCTTTAAAATGAACAGTCTGCAATCTAATGACAC
AGCCATATATTACTGTGCCAGAGCCCTCACCTACTATGATTACGAGTTTGCTTACTGGGGCCAAGGG
ACTCTGGTCACTGTCTCTAGCGGTGGAGGCGGTTCAGGCGGAGGTGGTTCCGGCGGTGGCGGCTCC
GACATCTTGCTGACTCAGTCTCCAGTCATCCTGTCTGTGAGTCCAGGAGAAAGAGTCAGTTTCTCCTG
CAGGGCCAGTCAGAGTATTGGCACAAACATACACTGGTATCAGCAAAGAACAAATGGTTCTCCAAG
GCTTCTCATAAAGTATGCTTCTGAGTCTATCTCTGGGATTCCTTCCAGGTTTAGTGGCAGTGGATCAG
GGACAGATTTTACTCTTAGCATCAACAGTGTGGAGTCTGAAGATATTGCAGATTATTACTGTCAACAA
AATAATAACTGGCCAACCACGTTCGGTGCTGGGACCAAGCTGGAGCTGAAACGT
SI-1X5 BISPECIFIC HEAVY CHAIN FULL-LENGTH AMINO ACID SEQUENCE (SEQ ID NO: 194).
HUMAN GAMMA-1 DOMAIN IS UNDERLINED (SEQ ID NO: 295), CONNECTOR IS IN ITALICS
(SEQ ID NO: 296), SCFV IS IN BOLD (SEQ ID NO: 297)
QVQLQESGGGLVKPGGSLRLSCAASGFTFSSYWMSWVRQAPGKGLEWVANINRDGSASYYVDSVKGR
FTISRDDAKNSLYLQMNSLRAEDTAVYYCARDRGVGYFDLWGRGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPS
NTKVDKRVEPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGKGGGGSGGGGSQVQLKQSGPGLVQPSQSLSITCTVSGFS
LTNYGVHWVRQSPGKGLEWLGVIWSGGNTDYNTPFTSRLSINKDNSKSQVFFKMNSLQSNDTAIYYC
ARALTYYDYEFAYWGQGTLVTVSSGGGGSGGGGSGGGGSDILLTQSPVILSVSPGERVSFSCRASQSIG
TNIHWYQQRTNGSPRLLIKYASESISGIPSRFSGSGSGTDFTLSINSVESEDIADYYCQQNNNWPTTFGA
GTKLELKR
SI-1X5 BISPECIFIC HEAVY CHAIN VARIABLE HEAVY CHAIN AMINO ACID SEQUENCE (SEQ ID
NO: 195). COMPLEMENTARITY DETERMINING REGIONS ARE UNDERLINED (SEQ ID NOS: 298,
299 AND 300)
QVQLQESGGGLVKPGGSLRLSCAASGFTFSSYWMSWVRQAPGKGLEWVANINRDGSASYYVDSVKGR
FTISRDDAKNSLYLQMNSLRAEDTAVYYCARDRGVGYFDLWGRGTLVTVSS
SI1X5 BISPECIFIC HEAVY CHAIN SCFV AMINO ACID SEQUENCE (SEQ ID NO: 196). ORDER: VH
(SEQ ID NO: 301)-LINKER (SEQ ID NO: 302)-VL (SEQ ID NO: 303). COMPLEMENTARITY
DETERMINING REGIONS ARE UNDERLINED (SEQ ID NOS: 304, 305, 306, 307, 308 AND 309).
LINKER IS IN BOLD ITALICS (SEQ ID NO: 302)
QVQLKQSGPGLVQPSQSLSITCTVSGFSLTNYGVHWVRQSPGKGLEWLGVIWSGGNTDYNTPFTSRLSI
NKDNSKSQVFFKMNSLQSNDTAIYYCARALTYYDYEFAYWGQGTLVTVSSGGGGSGGGGSGGGGSDIL
LTQSPVILSVSPGERVSFSCRASQSIGTNIHWYQQRTNGSPRLLIKYASESISGIPSRFSGSGSGTDFTLSINS
VESEDIADYYCQQNNNWPTTFGAGTKLELKR
SI-1X6 SEQUENCES
SI1X6 LIGHT CHAIN FULL-LENGTH NUCLEOTIDE SEQUENCE (SEQ ID NO: 197)
GACATCTTGCTGACTCAGTCTCCAGTCATCCTGTCTGTGAGTCCAGGAGAAAGAGTCAGTTTCTCCTG
CAGGGCCAGTCAGAGTATTGGCACAAACATACACTGGTATCAGCAAAGAACAAATGGTTCTCCAAG
GCTTCTCATAAAGTATGCTTCTGAGTCTATCTCTGGGATTCCTTCCAGGTTTAGTGGCAGTGGATCAG
GGACAGATTTTACTCTTAGCATCAACAGTGTGGAGTCTGAAGATATTGCAGATTATTACTGTCAACAA
AATAATAACTGGCCAACCACGTTCGGTGCTGGGACCAAGCTGGAGCTGAAACGTACGGTGGCTGCA
CCATCTGTCTTCATCTTCCCGCCATCTGATGAGCAGTTGAAATCTGGAACTGCCTCTGTTGTGTGCCTG
CTGAATAACTTCTATCCCAGAGAGGCCAAAGTACAGTGGAAGGTGGATAACGCCCTCCAATCGGGTA
ACTCCCAGGAGAGTGTCACAGAGCAGGACAGCAAGGACAGCACCTACAGCCTCAGCAGCACCCTGA
CGCTGAGCAAAGCAGACTACGAGAAACACAAAGTCTACGCCTGCGAAGTCACCCATCAGGGCCTGA
GCTCGCCCGTCACAAAGAGCTTCAACAGGGGAGAGTGT
SI-1X6 LIGHT CHAIN VARIABLE LIGHT CHAIN NUCLEOTIDE SEQUENCE (SEQ ID NO: 198)
GACATCTTGCTGACTCAGTCTCCAGTCATCCTGTCTGTGAGTCCAGGAGAAAGAGTCAGTTTCTCCTG
CAGGGCCAGTCAGAGTATTGGCACAAACATACACTGGTATCAGCAAAGAACAAATGGTTCTCCAAG
GCTTCTCATAAAGTATGCTTCTGAGTCTATCTCTGGGATTCCTTCCAGGTTTAGTGGCAGTGGATCAG
GGACAGATTTTACTCTTAGCATCAACAGTGTGGAGTCTGAAGATATTGCAGATTATTACTGTCAACAA
AATAATAACTGGCCAACCACGTTCGGTGCTGGGACCAAGCTGGAGCTGAAA
SI-1X6 LIGHT CHAIN FULL-LENGTH AMINO ACID SEQUENCE (SEQ ID NO: 199). HUMAN
KAPPA CONSTANT DOMAIN IS UNDERLINED (SEQ ID NO: 310)
DILLTQSPVILSVSPGERVSFSCRASQSIGTNIHWYQQRTNGSPRLLIKYASESISGIPSRFSGSGSGTDFTLSI
NSVESEDIADYYCQQNNNWPTTFGAGTKLELKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC
SI-1X6 LIGHT CHAIN VARIABLE LIGHT CHAIN AMINO ACID SEQUENCE (SEQ ID NO: 200).
COMPLEMENTARITY DETERMINING REGIONS ARE UNDERLINED (SEQ ID NOS: 311, 312 AND
313)
DILLTQSPVILSVSPGERVSFSCRASQSIGTNIHWYQQRTNGSPRLLIKYASESISGIPSRFSGSGSGTDFTLSI
NSVESEDIADYYCQQNNNWPTTFGAGTKLELK
SI1X6 BISPECIFIC HEAVY CHAIN FULL-LENGTH NUCLEOTIDE SEQUENCE (SEQ ID NO: 201)
CAGGTGCAGCTGAAGCAGTCAGGACCTGGCCTAGTGCAGCCCTCACAGAGCCTGTCCATCACCTGCA
CAGTCTCTGGTTTCTCATTAACTAACTATGGTGTACACTGGGTTCGCCAGTCTCCAGGAAAGGGTCTG
GAGTGGCTGGGAGTGATATGGAGTGGTGGAAACACAGACTATAATACACCTTTCACATCCAGACTG
AGCATCAACAAGGACAATTCCAAGAGCCAAGTTTTCTTTAAAATGAACAGTCTGCAATCTAATGACAC
AGCCATATATTACTGTGCCAGAGCCCTCACCTACTATGATTACGAGTTTGCTTACTGGGGCCAAGGG
ACTCTGGTCACTGTCTCTAGCGCTAGCACCAAGGGCCCATCGGTCTTCCCCCTGGCACCCTCCTCCAA
GAGCACCTCTGGGGGCACAGCGGCCCTGGGCTGCCTGGTCAAGGACTACTTCCCCGAACCGGTGAC
GGTGTCGTGGAACTCAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCACCCAGACCTACATCT
GCAACGTGAATCACAAGCCCAGCAACACCAAGGTGGACAAGAGAGTTGAGCCCAAATCTTGTGACA
AAACTCACACATGCCCACCGTGCCCAGCACCTGAACTCCTGGGGGGACCGTCAGTCTTCCTCTTCCCC
CCAAAACCCAAGGACACCCTCATGATCTCCCGGACCCCTGAGGTCACATGCGTGGTGGTGGACGTGA
GCCACGAAGACCCTGAGGTCAAGTTCAACTGGTACGTGGACGGCGTGGAGGTGCATAATGCCAAGA
CAAAGCCGCGGGAGGAGCAGTACAACAGCACGTACCGTGTGGTCAGCGTCCTCACCGTCCTGCACC
AGGACTGGCTGAATGGCAAGGAGTACAAGTGCAAGGTCTCCAACAAAGCCCTCCCAGCCCCCATCG
AGAAAACCATCTCCAAAGCCAAAGGGCAGCCCCGAGAACCACAGGTGTACACCCTGCCCCCATCCCG
GGAGGAGATGACCAAGAACCAGGTCAGCCTGACCTGCCTGGTCAAAGGCTTCTATCCCAGCGACAT
CGCCGTGGAGTGGGAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGGTGGCAGCAGGGGAA
CGTCTTCTCATGCTCCGTGATGCATGAGGCTCTGCACAACCACTACACGCAGAAGAGCCTCTCCCTGT
CTCCGGGTAAAGGCGGTGGAGGATCCGGCGGTGGTGGATCACAGGTGCAGCTGCAGGAGTCGGGG
GGAGGCCTGGTCAAGCCTGGAGGGTCCCTGAGACTCTCCTGTGCAGCCTCTGGATTCACCTTTAGTA
GTTATTGGATGAGCTGGGTCCGCCAGGCTCCAGGGAAGGGGCTGGAGTGGGTGGCCAACATAAAC
CGCGATGGAAGTGCGAGTTACTATGTGGACTCTGTGAAGGGCCGATTCACCATCTCCAGAGACGAC
GCCAAGAACTCACTGTATCTGCAAATGAACAGCCTGAGAGCTGAGGACACGGCTGTGTATTACTGTG
CGAGAGATCGTGGGGTGGGCTACTTCGATCTCTGGGGCCGTGGCACCCTGGTCACCGTCTCGAGCG
GTGGAGGCGGTTCAGGCGGAGGTGGTTCCGGCGGTGGCGGCTCCCAGTCTGCCCTGACTCAGCCTG
CCTCCGTGTCTGGGTCTCCTGGACAGTCGATCACCATCTCCTGCACTGGAACCAGCAGTGACGTTGGT
GGTTATAACTTTGTCTCCTGGTACCAACAACACCCAGGCAAAGCCCCCAAACTCATGATCTATGATGT
CAGTGATCGGCCCTCAGGGGTGTCTGATCGCTTCTCCGGCTCCAAGTCTGGCAACACGGCCTCCCTG
  ATCATCTCTGGCCTCCAGGCTGACGACGAGGCTGATTATTACTGCAGCTCATATGGGAGCAGCAGCA
CTCATGTGATTTTCGGCGGAGGGACCAAGGTGACCGTCCTA
SI-1X6 BISPECIFIC HEAVY CHAIN VARIABLE HEAVY CHAIN NUCLEOTIDE SEQUENCE (SEQ ID
NO: 202)
CAGGTGCAGCTGAAGCAGTCAGGACCTGGCCTAGTGCAGCCCTCACAGAGCCTGTCCATCACCTGCA
CAGTCTCTGGTTTCTCATTAACTAACTATGGTGTACACTGGGTTCGCCAGTCTCCAGGAAAGGGTCTG
GAGTGGCTGGGAGTGATATGGAGTGGTGGAAACACAGACTATAATACACCTTTCACATCCAGACTG
AGCATCAACAAGGACAATTCCAAGAGCCAAGTTTTCTTTAAAATGAACAGTCTGCAATCTAATGACAC
AGCCATATATTACTGTGCCAGAGCCCTCACCTACTATGATTACGAGTTTGCTTACTGGGGCCAAGGG
ACTCTGGTCACTGTCTCTAGC
SI-1X6 BISPECIFIC HEAVY CHAIN SCFV NUCLEOTIDE SEQUENCE (SEQ ID NO: 203)
CAGGTGCAGCTGCAGGAGTCGGGGGGAGGCCTGGTCAAGCCTGGAGGGTCCCTGAGACTCTCCTGT
GCAGCCTCTGGATTCACCTTTAGTAGTTATTGGATGAGCTGGGTCCGCCAGGCTCCAGGGAAGGGG
CTGGAGTGGGTGGCCAACATAAACCGCGATGGAAGTGCGAGTTACTATGTGGACTCTGTGAAGGGC
CGATTCACCATCTCCAGAGACGACGCCAAGAACTCACTGTATCTGCAAATGAACAGCCTGAGAGCTG
AGGACACGGCTGTGTATTACTGTGCGAGAGATCGTGGGGTGGGCTACTTCGATCTCTGGGGCCGTG
GCACCCTGGTCACCGTCTCGAGCGGTGGAGGCGGTTCAGGCGGAGGTGGTTCCGGCGGTGGCGGC
TCCCAGTCTGCCCTGACTCAGCCTGCCTCCGTGTCTGGGTCTCCTGGACAGTCGATCACCATCTCCTG
CACTGGAACCAGCAGTGACGTTGGTGGTTATAACTTTGTCTCCTGGTACCAACAACACCCAGGCAAA
GCCCCCAAACTCATGATCTATGATGTCAGTGATCGGCCCTCAGGGGTGTCTGATCGCTTCTCCGGCTC
CAAGTCTGGCAACACGGCCTCCCTGATCATCTCTGGCCTCCAGGCTGACGACGAGGCTGATTATTACT
GCAGCTCATATGGGAGCAGCAGCACTCATGTGATTTTCGGCGGAGGGACCAAGGTGACCGTCCTA
SI-1X6 BISPECIFIC HEAVY CHAIN FULL-LENGTH AMINO ACID SEQUENCE (SEQ ID NO: 204).
HUMAN GAMMA-1 DOMAIN IS UNDERLINED (SEQ ID NO: 314), CONNECTOR IS IN ITALICS
(SEQ ID NO: 315), SCFV IS IN BOLD (SEQ ID NO: 316)
QVQLKQSGPGLVQPSQSLSITCTVSGFSLTNYGVHWVRQSPGKGLEWLGVIWSGGNTDYNTPFTSRLSI
NKDNSKSQVFFKMNSLQSNDTAIYYCARALTYYDYEFAYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQ
QGNVFSCSVMHEALHNHYTQKSLSLSPGKGGGGSGGGGSQVQLQESGGGLVKPGGSLRLSCAASGFT
FSSYWMSWVRQAPGKGLEWVANINRDGSASYYVDSVKGRFTISRDDAKNSLYLQMNSLRAEDTAVY
YCARDRGVGYFDLWGRGTLVTVSSGGGGSGGGGSGGGGSQSALTQPASVSGSPGQSITISCTGTSSD
VGGYNFVSWYQQHPGKAPKLMIYDVSDRPSGVSDRFSGSKSGNTASLIISGLQADDEADYYCSSYGSS
STHVIFGGGTKVTVL
SI-1X6 BISPECIFIC HEAVY CHAIN VARIABLE HEAVY CHAIN AMINO ACID SEQUENCE (SEQ ID
NO: 205). COMPLEMENTARITY DETERMINING REGIONS ARE UNDERLINED (SEQ ID NOS: 317,
318 AND 319)
QVQLKQSGPGLVQPSQSLSITCTVSGFSLTNYGVHWVRQSPGKGLEWLGVIWSGGNTDYNTPFTSRLSI
NKDNSKSQVFFKMNSLQSNDTAIYYCARALTYYDYEFAYWGQGTLVTVSS
SI1X6 BISPECIFIC HEAVY CHAIN SCFV AMINO ACID SEQUENCE (SEQ ID NO: 206). ORDER: VH
(SEQ ID NO: 320)-LINKER (SEQ ID NO: 321)-VL (SEQ ID NO: 322). COMPLEMENTARITY
DETERMINING REGIONS ARE UNDERLINED (SEQ ID NOS: 323, 324, 325, 326, 327 AND 328).
LINKER IS IN BOLD ITALICS (SEQ ID NO: 321)
QVQLQESGGGLVKPGGSLRLSCAASGFTFSSYWMSWVRQAPGKGLEWVANINRDGSASYYVDSVKGR
FTISRDDAKNSLYLQMNSLRAEDTAVYYCARDRGVGYFDLWGRGTLVTVSSGGGGSGGGGSGGGGSQ
SALTQPASVSGSPGQSITISCTGTSSDVGGYNFVSWYQQHPGKAPKLMIYDVSDRPSGVSDRFSGSKSGN
TASLIISGLQADDEADYYCSSYGSSSTHVIFGGGTKVTVL
```

1-62. (canceled)

63. A bispecific tetravalent antibody, said bispecific tetravalent antibody comprising:
two IgG1 heavy chains;
two kappa light chains; and
two single chain Fv (scFv) domains;
wherein the two lgGl heavy chains and kappa light chains form an lgG moiety having binding specificity to a first member of the EGFR family;
wherein the two scFv domains have binding specificity to a second member of the EGFR family.

64. The bispecific tetravalent antibody of claim 63, wherein the IgG1 heavy chain comprises a heavy chain complementarity-determining region 1 (CDR1) comprising the amino acid sequence of NYGVH (SEQ ID NO:317), a heavy chain complementarity-determining region 2 (CDR2) comprising the amino acid sequence of VIWSGGNTDYNTPFTS (SEQ ID NO: 318), and a heavy chain complementarity-determining region 3 (CDR3) comprising the amino acid sequence of ALTYYDYEFAY (SEQ ID NO:319); and
wherein the kappa light chain comprises a light chain CDR1 comprising the amino acid sequence of RASQSIGTNIH (SEQ ID NO:311), a light chain CDR2 comprising the amino acid sequence of YASESIS (SEQ ID NO:312), a light chain CDR3 comprising the amino acid sequence of QQNNNWPTT (SEQ ID NO:313).

65. The bispecific tetravalent antibody of claim 63, wherein the IgG1 heavy chain comprises a heavy chain variable region comprising the amino acid sequence of SEQ ID NO:137 or 119; and wherein the kappa light chain comprises a light chain variable region comprising the amino acid sequence of SEQ ID NO:132 or 114.

66. The bispecific tetravalent antibody of claim 63, wherein the scFv comprises a heavy chain CDR1 comprising the amino acid sequence of SYWMS (SEQ ID NO:323), a heavy chain CDR2 comprising the amino acid sequence of NINRDGSASYYVDSVKG (SEQ ID NO: 324), a heavy chain CDR3 comprising the amino acid sequence of DRGVGYFDL (SEQ ID NO: 325), a light chain CDR1 comprising the amino acid sequence of TGTSSDVGGYNFVS (SEQ ID NO:326), a light chain CDR2 comprising the amino acid sequence of DVSDRPS (SEQ ID NO: 327), and a light chain CDR3 comprising the amino acid sequence of SSYGSSSTHVI (SEQ ID NO:328).

67. The bispecific tetravalent antibody of claim 63, wherein the scFv comprises a heavy chain variable region comprising the amino acid sequence of QVQLQESGGGLVKPGGSLRLSCAASGFTFSSYWMSWVRQAPGKGLEWVANINRDGSA SYYVDSVKGRFTISRDDAKNSLYLQMNSLRAEDTAVYYCARDRGVGYFDLWGRGTLV TVSS (SEQ ID NO:320); and a light chain variable region comprising the amino acid sequence of
(SEQ ID NO: 322)
QSALTQPASVSGSPGQSITISCTGTSSDVGGYNFVSWYQQHPGKAPKLM
IYDVSDRPSGVSDRFSGSKSGNTASLIISGLQADDEADYYCSSYGSSST
HVIFGGGTKVTVL.

68. The bispecific tetravalent antibody of claim 63, wherein the IgG1 heavy chain comprises a heavy chain CDR1 comprising the amino acid sequence of NYGVH (SEQ ID NO: 317), a heavy chain CDR2 comprising the amino acid sequence of VIWSGGNTDYNTPFTS (SEQ ID NO:318), and a heavy chain CDR3 comprising the amino acid sequence of ALTYYDYEFAY (SEQ ID NO:319), and wherein the kappa light chain comprises a light chain CDR1 comprising the amino acid sequence of RASQSIGTNIH (SEQ ID NO:311), a light chain CDR2 comprising the amino acid sequence of YASESIS (SEQ ID NO:312), a light chain CDR3 comprising the amino acid sequence of QQNNNWPTT (SEQ ID NO:313); and
wherein the scFv comprises a heavy chain CDR1 comprising the amino acid sequence of SYWMS (SEQ ID NO:323), a heavy chain CDR2 comprising the amino acid sequence of NINRDGSASYYVDSVKG (SEQ ID NO:324), a heavy chain CDR3 comprising the amino acid sequence of DRGVGYFDL (SEQ ID NO:325), a light chain CDR1 comprising the amino acid sequence of TGTSSDVGGYNFVS (SEQ ID NO:326), a light chain CDR2 comprising the amino acid sequence of DVSDRPS (SEQ ID NO:327), and a light chain CDR3 comprising the amino acid sequence of SSYGSSSTHVI (SEQ ID NO:328).

69. The bispecific tetravalent antibody of claim 63, wherein the IgG1 heavy chain comprises a heavy chain variable region comprising the amino acid sequence of SEQ ID NO:137 or 119; and wherein the kappa light chain comprises a light chain variable region comprising the amino acid sequence of SEQ ID NO:132 or 114, wherein the scFv comprises a heavy chain variable region comprising the amino acid sequence of
(SEQ ID NO: 320)
QVQLQESGGGLVKPGGSLRLSCAASGFTFSSYWMSWVRQAPGKGLEWVA
NINRDGSASYYVDSVKGRFTISRDDAKNSLYLQMNSLRAEDTAVYYCAR
DRGVGYFDLWGRGTLVTVSS;
and a light chain variable region comprising the amino acid sequence of
(SEQ ID NO: 322)
QSALTQPASVSGSPGQSITISCTGTSSDVGGYNFVSWYQQHPGKAPKLM
IYDVSDRPSGVSDRFSGSKSGNTASLIISGLQADDEADYYCSSYGSSST
HVIFGGGTKVTVL.

70. The bispecific tetravalent antibody of claim 63, wherein each of the two scFv domains is connected to the C-terminus of each of the IgG1 heavy chains by a connector.

71. The bispecific tetravalent antibody of claim 70, wherein the connector comprises the amino acid sequence of (gly-gly-gly-gly-ser) n (SEQ ID NO:329), wherein n is an integral of at least 1.

72. The bispecific tetravalent antibody of claim 63, the scFv domain has a structure order of N terminus-heavy chain variable region-linker-light chain variable region-C terminus, or N-terminus-light chain variable region-linker-heavy chain variable region-C-terminus

73. The bispecific tetravalent antibody of claim 72, wherein the linker comprises the amino acid sequence of (gly-gly-gly-gly-ser) m (SEQ ID NO:321), wherein m is an integral of at least 3.

74. The bispecific tetravalent antibody of claim 63, wherein the IgG1 heavy chain is humanized or human IgG1 heavy chain.

75. The bispecific tetravalent antibody of claim 63, wherein the kappa light chain is humanized or human kappa light chain.

76. The bispecific tetravalent antibody of claim 63, wherein the IgG1 heavy chain, connector, and scFv domain comprises the amino acid sequence of SEQ ID NO: 136, and the kappa light chain comprises the amino acid sequence of SEQ ID NO: 131.

77. An isolated nucleic acid encoding the antibody of claim 63.

78. An expression vector comprising the isolated nucleic acid of claim 77.

79. The expression vector of claim 78, wherein the vector is expressible in a cell.

80. A host cell comprising the nucleic acid of claim 77.

81. A host cell comprising the expression vector of claim 78.

82. The host cell of claim 80, wherein the host cell is a prokaryotic cell or a eukaryotic cell.

83. An immunoconjugate comprising the antibody of claim 63 and a cytotoxic agent.

84. A pharmaceutical composition, comprising the bispecific tetravalent antibody of claim 63 and a pharmaceutically acceptable carrier.

85. The pharmaceutical composition of claim 84, further comprising radioisotope, radionuclide, a toxin, a therapeutic agent, a chemotherapeutic agent or a combination thereof.

86. A pharmaceutical composition, comprising the immunoconjugate of claim 83 and a pharmaceutically acceptable carrier.

87. A method of treating cancer in a subject, comprising administering to the subject an effective amount of the bispecific tetravalent antibody of claim 63.

88. A method of inhibiting the biological activity of a HER receptor in a subject, comprising administering to the subject an effective amount of the antibody of claim 63 to inhibit a biological activity of said HER receptor, wherein said HER receptor is EGFR and/or HER3.
